SEQUENCE LISTING (19 sequences)

SEQ ID NO: 1 - DNA, 339 bp, synthetic construct
  source 1..339: mol_type = other DNA
agggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 2 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 3 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 4 - protein, 42 aa, synthetic construct
KRGRKKLLYIFKQPFMRPVQTTQEEDGCSCRFPEEEEGGCEL

SEQ ID NO: 5 - DNA, 63 bp, synthetic construct
  source 1..63: mol_type = other DNA
atggccttaccagtgaccgccttgctcctgccgctggccttgctgctccacgccgccaggccg

SEQ ID NO: 6 - protein, 21 aa, synthetic construct
MALPVTALLLPLALLLHAARP

SEQ ID NO: 7 - DNA, 738 bp, synthetic construct
  source 1..738: mol_type = other DNA
gacgtgcacctgcaggagagcggccccgacctggtgaagcccagccagagcctgagcctgacctgcaccgtgaccggctacagcatcaccagcggctacacctggcactggatcaggcagttccccggcaacaccgtggagtggatgggctacatccagtacaccggcagcaccaggtacaaccccgccctgaggggcaggctgagcatcagcagggacaccagcaagaaccagttcttcctgcagctgatcagcgtgaccaccgccgacaccggcacctacttctgcgccaggggcaccaccgccagcttcgactactggggccagggcaccaccctgaccgtggccagcgccggcggcggcggcagcggcggcggcggcagcggcggcggcggcagcgacatcgtgatgagccagagccccagcagcctgaacgtgagcgtgggcgagaaggtgaccatgaggtgcaggagcagccagagcctgctgtacagcagcgaccagaagaactacctgacctggtaccagcagaagcccggccagagccccaagctgctgatcagctgggccagcaccagggccagcggcgtgcccgacaggttcaccggcagcggcagcggcaccgacttcaccctgaccatcagcagcgtgaaggccgaggacctgggcgtgtactactgccagcagtacttcatctaccccctgaccttcggcgtgggcaccaagctgggcctgaag

SEQ ID NO: 8 - protein, 246 aa, synthetic construct
DVHLQESGPDLVKPSQSLSLTCTVTGYSITSGYTWHWIRQFPGNTVEWMGYIQYTGSTRYNPALRGRLSISRDTSKNQFFLQLISVTTADTGTYFCARGTTASFDYWGQGTTLTVASAGGGGSGGGGSGGGGSDIVMSQSPSSLNVSVGEKVTMRCRSSQSLLYSSDQKNYLTWYQQKPGQSPKLLISWASTRASGVPDRFTGSGSGTDFTLTISSVKAEDLGVYYCQQYFIYPLTFGVGTKLGLK

SEQ ID NO: 9 - DNA, 207 bp, synthetic construct
  source 1..207: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 10 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 11 - DNA, 165 bp, synthetic construct
  source 1..165: mol_type = other DNA
gcctgcccctacagcaaccccagcctgtgcagcggcggcggcggcagcgagctgcccacccagggcaccttcagcaacgtgagcaccaacgtgagccccgccaagcccaccaccaccgcctgtccttattccaatccttccctgtgtagcggcggcggcggcagc

SEQ ID NO: 12 - protein, 55 aa, synthetic construct
ACPYSNPSLCSGGGGSELPTQGTFSNVSTNVSPAKPTTTACPYSNPSLCSGGGGS

SEQ ID NO: 13 - DNA, 57 bp, synthetic construct
  source 1..57: mol_type = other DNA
gccaccaacttcagcctgctgaagcaggccggcgacgtggaggagaaccccggcccc

SEQ ID NO: 14 - protein, 19 aa, synthetic construct
ATNFSLLKQAGDVEENPGP

SEQ ID NO: 15 - DNA, 408 bp, synthetic construct
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 16 - DNA, 729 bp, synthetic construct
  source 1..729: mol_type = other DNA
gacatcgtgctgacccagagcccccccagcctggccatgagcctgggcaagagggccaccatcagctgcagggccagcgagagcgtgaccatcctgggcagccacctgatccactggtaccagcagaagcccggccagccccccaccctgctgatccagctggccagcaacgtgcagaccggcgtgcccgccaggttcagcggcagcggcagcaggaccgacttcaccctgaccatcgaccccgtggaggaggacgacgtggccgtgtactactgcctgcagagcaggaccatccccaggaccttcggcggcggcaccaagctggagatcaagggaggggggggatccgggggaggaggctccggcggaggcggaagccagatccagctggtgcagagcggccccgagctgaagaagcccggcgagaccgtgaagatcagctgcaaggccagcggctacaccttcaccgactacagcatcaactgggtgaagagggcccccggcaagggcctgaagtggatgggctggatcaacaccgagaccagggagcccgcctacgcctacgacttcaggggcaggttcgccttcagcctggagaccagcgccagcaccgcctacctgcagatcaacaacctgaagtacgaggacaccgccacctacttctgcgccctggactacagctacgccatggactactggggccagggcaccagcgtgaccgtgagcagc

SEQ ID NO: 17 - protein, 243 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 18 - protein, 80 aa, synthetic construct
MGRAMVARLGLGLLLLALLLPTQIYSSETTTGTSSNSSQSTSNSGLAPNPTNATTKAAGGALQSTASLFVVSLSLLHLYS

SEQ ID NO: 19 - protein, 8 aa, synthetic construct
  VARIANT 2: note = X=V or I
DXEXNPGP

(19) United States
(12) Patent Application Publication
(10) Pub. No.: US 2026/0364109 A1
(43) Pub. Date: Nov. 19, 2026

(54) ANTI-CD24 CHIMERIC ANTIGEN RECEPTOR, COMPOSITIONS AND METHODS

(71) Applicant: BioVentures, LLC, Little Rock, AR (US)

(72) Inventors: Fenghuang ZHAN, LITTLE ROCK, AR (US); Fumou SUN, LITTLE ROCK, AR (US)

(21) Appl. No.: 19/487,210

(22) PCT Filed: May 22, 2024
(86) PCT No.: PCT/US2024/030577

§ 371 Date: Nov. 24, 2025

Related U.S. Application Data
(60) Provisional application No. 63/469,293, filed on May 26, 2023.

(51) Int. Cl. A61K 35/17; A61K 40/11; A61K 40/31; A61K 40/42; A61P 35/00; C07K 14/705; C07K 16/28; C12N 5/0783

(52) U.S. Cl. CPC A61K 35/17; A61K 40/11; A61K 40/31; A61K 40/4224; A61P 35/00; C07K 14/7051; C07K 16/2878; C07K 16/2896; C12N 5/0636; A61K 2239/13; A61K 2239/29; C07K 2317/31; C07K 2317/622; C12N 2510/00

(57) ABSTRACT

The disclosure relates to immune cellular therapy. In particular, the disclosure relates to chimeric antigen receptor (CAR) constructs, cells comprising the same, and methods of treating subjects in need thereof. Specifically, the disclosure provides CAR constructs comprising a first antigen recognition domain which binds to protein CD24 or B-cell maturation antigen (BCMA), and wherein the CAR further comprising a second antigen recognition domain which binds to BCMA or CD24, and wherein the first antigen and second antigen recognition domain are in tandem. Further disclosed are methods of using the immune cells comprising the CAR for treating different types of cancers.

### CROSS REFERENCE TO RELATED APPLICATIONS

The present application claims the benefit of U.S. Provisional Application No. 63/469,293, entitled, “ANTI-CD24 CHIMERIC ANTIGEN RECEPTOR, COMPOSITIONS AND METHODS” filed May 26, 2023, the content of which is hereby incorporated by reference in its entirety.

### INCORPORATION OF SEQUENCE LISTING

The present application contains a Sequence Listing which has been submitted in .XML format via Patent Center and is hereby incorporated by reference in its entirety. Said WIPO Sequence Listing was created on May 16, 2024, is named 052592_797706_SequenceListing.xml, and is 21 kilobytes in size.

### FIELD OF THE TECHNOLOGY

This application generally relates to immune cellular therapy. In particular, the disclosure relates to chimeric antigen receptor (CAR) constructs, cells comprising the same, and methods of treating subjects in need thereof.

### BACKGROUND

BCMA-targeting CAR-T has become an effective therapeutic approach for multiple myeloma (MM). FDA-approved BCMA-CAR-T cell therapy has shown impressive overall response rates. However, the durability of these responses is limited and even patients with initial complete responses eventually relapse. The median progression-free survival of BCMA-CAR T cell therapy (idecabtagene vicleucel, Bristol Myers Squibb) was 8.8 months. Relapses may be due to several factors, including loss of BCMA expression or tumor-initiating cells (TICs) that survive and cause relapses. To mitigate these limitations, combining BCMA-CAR T cells with different targets and bispecific CAR-T cells have been introduced. CD19-CAR-T cell and BCMA-CAR-T cell combination therapy induced durable responses in patients with refractory/relapsed multiple myeloma. Although expression of CD19 is rare in plasma cells, there is a small population of CD19⁺ MM cells that have properties of TICs. These have been associated with high-risk disease, poor prognosis, early relapse, and reduced survival.

Thus, there is a need to identify new and efficient targets for CAR-T therapy for treating multiple myeloma, and other B cell and/or plasma cell malignancies.

### SUMMARY

In some aspects, the present disclosure encompasses an immune effector cell comprising a chimeric antigen receptor, wherein chimeric antigen receptor comprises a first antigen recognition domain which binds to CD24 or B-cell maturation antigen (BCMA).

In further aspects, the disclosure provides an immune effector cell comprising a chimeric antigen receptor, wherein chimeric antigen receptor comprises a first antigen recognition domain which specifically binds to CD24. In some aspects, the chimeric antigen receptor further comprises a second antigen recognition domain. In some aspects, the second antigen recognition domain specifically binds to B-cell maturation antigen (BCMA). In some aspects, the first antigen and second antigen recognition domain are in tandem.

In certain aspects, provided herein is an immune effector cell comprising a chimeric antigen receptor, wherein chimeric antigen receptor comprises a first antigen recognition domain which specifically binds to BCMA. In some aspects, the chimeric antigen receptor further comprises a second antigen recognition domain. In some aspects, the second antigen recognition domain which binds to CD24. In some aspects, the first antigen recognition domain, and the second antigen recognition domain further comprise a transmembrane domain, an intracellular domain, or any combination thereof.

In some aspects, the antigen recognition domain which specifically binds to CD24 comprises an scFv with a nucleic acid sequence having at least 70%, 80%, 90%, or 100% sequence identity with SEQ ID NO: 7 or an amino acid sequence having at least 70%, 80%, 90%, or 100% sequence identity with SEQ ID NO: 8. In some aspects, the antigen recognition domain which specifically binds to BCMA comprises an scFv with a nucleic acid sequence having at least 70%, 80%, 90%, or 100% sequence identity with SEQ ID NO: 16 or an amino acid sequence having at least 70%, 80%, 90%, or 100% sequence identity with SEQ ID NO: 17.

In some aspects, the disclosed chimeric antigen receptor further comprises a suicide gene. In some aspects, the suicide gene comprises a RQR8. In some aspects, the suicide gene comprises a modified RQR8 comprising at least one CD20 mimotope, at least one CD34 epitope, or any combination thereof.

In some aspects, the disclosed immune effector cell is selected from the group consisting of a T cell, a Natural Killer (NK) cell, a cytotoxic T lymphocyte (CTL), and a regulatory T cell, and any combination thereof.

In some aspects, the disclosure further provides a composition comprising an immune effector cell or a population of immune effector cells disclosed herein, and a pharmaceutically acceptable carrier.

Further provided is a method of treating a malignancy in a subject in need thereof, the method comprising administering to the subject composition disclosed herein.

In some aspects, the malignancy is a B cell malignancy or a plasma cell malignancy. In some aspects, the malignancy is a Burkitt lymphoma, chronic lymphocytic leukemia/small lymphocytic lymphoma (CLL/SLL), diffuse large B-cell lymphoma, follicular lymphoma, or mantle cell lymphoma, Monoclonal gammopathy of undetermined significance, Lymphoplasmacytic lymphoma, Plasmacytoma, or myeloma. In some aspects, the malignancy is myeloma. In further aspects, the malignancy is a relapsed or refractory malignancy.

In some aspects, administering the population of immune effector cell reduces the tumor burden in the subject by at least 10%. In some aspects, administering the population of immune effector cell increases survivability of the subject by at least 10%.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-1D show proportion of CD24-positive cells is increased in MM after treatment failure. FIG. 1A shows the percentage of CD24⁺ monoclonal plasma cells were compared in samples from patients with monoclonal gammopathy of undetermined significance/smoldering MM (MGUS/SMM), patients with newly diagnosed MM (NDMM), and 21 patients after treatment with relapsed/refractory MM (RRMM). The mean percentage of CD24⁺/CD138⁺/CD38⁺/CD45-plasma cells was increased after treatment. One-way ANOVA was used. FIG. 1B shows flow cytometry analysis of patient samples and the association between drug response and CD24 expression in MM cells. The percentage of the subpopulation of CD138⁺CD24⁺ cells increase in 12 of 15 primary myeloma samples post-bortezomib treatment. Paired t-test was used. FIG. 1C shows flow cytometry analysis of CD24 expression in 20 MM cell lines. All the MM cell lines expressed CD24. Some cell lines highly expressed CD24, such as OPM2, MM1-144, and KMS-34. Fluorescence minus one (FMO) control were samples stained with all of the fluorophores, minus CD24-APC-CY7. FIG. 1D shows bar plot showing expression levels of CD24 in 20 MM cell lines. * p<. 05, *** p<. 001.

FIG. 2A-2I show CD24-CAR-T cells target MM cells and promote macrophage phagocytic clearance in vitro. FIG. 2A shows CD24-CAR-T and MOCK-CAR-T constructs. CD24-CAR vector was constructed by CD24-specific single-chain variable fragments (scFvs), a safety switch (RQR8) in the hinge region, and a 4-1BB co-activation domain with CD37. The MOCK-CAR vector only contains a safety switch in the hinge region and a 4-1BB co-activation domain with CD37. FIG. 2B shows the phenotype of CAR-T cells, including the ratio of CD4 and CD8 phenotypes and the ratio of different memory phenotypes. Memory phenotypes were assigned according to CD62L and CD45RO expression within the CAR-T cell population as follows: stem cell memory (CD45RO−/CD62L+), central memory (CD45RO+/CD62L+), effector memory (CD45RO+/CD62L−), effector cells (CD45RO−/CD62L−). FIG. 2C shows CD24-CAR-T cells cytolytic activity in vitro. CAR-T or T cells were added to MM1.S, OMP2, and H929 cell lines at the effector/target (E/T) ratio from 1:5 to 5:1. After 24 hours of coculture, cytolytic activity was measured. FIG. 2D shows the expression of T cell activation marker CD69 was detected at the E/T ratio was 5:1 after 24 hours of coculture. FIG. 2E shows Interleukin (IL)-2 concentrations on supernatants were detected at the E/T ratio was 5:1 after 24 hours of coculture. FIG. 2F shows Interferon (IFN)-γ concentrations on supernatants were detected at the E/T ratio was 5:1 after 24 hours of coculture. FIG. 2G shows sixteen MM patient samples with CD24-CAR-T cells or PBS treatment for 24 hours. The percentage of the subpopulation of CD138⁺ cells decreased in 16 of 16 primary myeloma samples post-CD24-CART treatment (top). A representative example of flow cytometry analysis is also presented (bottom). FIG. 2H shows fluorescent images of phagocytic clearance. Phagocytosis was performed by coculture of OPM2 cells that expressed GFP (green), DiD-stained macrophages (red), and CAR-T cells at a ratio of 2:1:1. After a 4-hour coculture, suspended cells were washed and detected. FIG. 2I shows bar plot showing the percentage of phagocytosis detected by flow cytometry analysis. One-way ANOVA was used in FIG. 2C, FIG. 2D, FIG. 2E, FIG. 2F, FIG. 2I and paired t-test was used in (g). * p<01, *p<. 001, ns=p>0.05.

FIG. 3A-3C illustrate generation of CD24-CAR-T cells. FIG. 3A illustrates lentivirus combined with the RetroNectin transfection technology used to modify the CD3⁺ T cells from healthy donor peripheral blood mononuclear cells (PBMCs). CAR-T cells were detected with flow cytometry and the RQR8-specific CD34 antibody. The CD34 positive rates were 43% for CD24-CAR-T cells. FIG. 3B shows CD34 Microbeads were used to isolate the CAR-T cells. After isolation, the CD34 positive rate increased to 83.6% for CD24-CAR-T cells. FIG. 3C shows CCK8 kits were used to detect CAR-T cell proliferation.

FIG. 4 shows CD24-CAR-T cells release TNF-α in vitro. TNF-α concentrations on supernatants were detected when the E/T ratio was 5:1 after 24 hours of coculture. One-way ANOVA was used for statistical analysis. *** p<001.

FIG. 5A-5C show CD24-CAR-T cells eliminate MM cells in vivo. FIG. 5A shows flow cytometry analysis of CD24 expression in 5TGM1 cell line by anti-mouse CD24 antibody (dark blue). The SWA11 antibody could target the CD24 in 5TGM1 cells (yellow). FIG. 5B shows bar plot showing the percentage of MM cells (GFP⁺ cells) in 5TGM1 BM samples. One-way ANOVA was used for statistical analysis. *p<. 05, *** p<. 001, ns=p>0.05. FIG. 5C shows mouse weight normalized to day 0 for each group, from the day after 5TGM1 MM cells injection. One-way ANOVA was used for statistical analysis. ns=p>0.05.

FIG. 6A-6G shows CD24-CAR-T cells target MM cells and promote macrophage phagocytic clearance in vivo. FIG. 6A illustrates schematic approach for 5TGM1 mouse model. Female C57BL/KaLwRij mice were intravenously injected with either PBS or 5TGM1-GFP-DiD cells. After 7 days after injection of 5TGM1 cells, mice were treated with either PBS, MOCK-CAR-T cells, or CD24-CAR-T cells. On day 21 after 5TGM1-cell inoculation, mice were killed. Serum electrophoresis (SPE) was performed. Bone marrow mononuclear cells (BMMCs) were isolated. Dormant (GFP⁺DiD^{Hi}) and activated (GFP⁺DiD^{Ne}g) cells were detected by flow cytometry. BM microenvironmental cells were sorted out for single-cell RNA sequencing (scRNA-seq). FIG. 6B shows SPE of 5TGM1 models. The M-spike is indicated in red. FIG. 6C shows representative GFP⁺DiD^{Hi} and GFP⁺DiD^{Ne}g gating strategy to identify dormant and activated MM cell populations in 5TGM1 BM samples. Bar plot showing the percentage of dormant cells in BM (right). FIG. 6D shows representative F4/80⁺CD11b⁺ gating strategy to identify the population of MM cells phagocytosed by macrophages. Bar plot showing the percentage of GFP⁺ cells phagocytosed by macrophages (right). FIG. 6E shows representative CD206⁺ and CD86⁺ gating strategy to identify M2-phenotype and M1-phenotype cell populations in 5TGM1 BM samples. FIG. 6F is a bar plot showing the percentage of M1-phenotype (left) and M2-phenotype macrophages (right) after treatment. FIG. 6G shows kaplan-Meier survival analysis of CAR-T treatment in 5TGM1 models. One-way ANOVA was used for statistical analysis. * p<05, ** p<01, *** p<. 001, ns=p>0.05.

FIG. 7A-7H show scRNA-seq revealed tumor microenvironment modification after CD24-CAR-T cells treatment. FIG. 7A shows UMAP plot of whole BMMCs. Whole BMMCs were divided into 16 subclusters (Neutrophils-I, Neutrophils-II, Myelocytes-I, Myelocytes-II, Myeloblasts, Monoblasts-I, Monoblasts-II, Basophils, dendritic cells [DCs], Monocytes, Macrophages, Natural killer/T cells [NK/T], Pro-B, Pre-B-I, Pre-B-II, Erythroblasts). FIG. 7B shows dot plot showed the main expression of marker genes across different immune cell types in mouse BM. FIG. 7C shows UMAP plot of BMMCs derived from healthy mice (n=3), MM-bearing mice treated with PBS (n=3), MOCK-CAR-T (n=3) or CD24 CAR-T (n=3). FIG. 7D display bar-views showing the proportion of various cell types in BMMCs of healthy and MM-bearing mice treated with PBS, MOCK-CAR-T, or CD24-CAR-T cells. FIG. 7E display bar-views showing the proportion of macrophages in whole BMMCs. FIG. 7F shows UMAP plot of macrophages subcluster. Macrophages were divided into 5 subclusters. FIG. 7G shows KEGG pathway analysis of dramatically changed subcluster 0 of macrophages. FIG. 7H shows KEGG pathway analysis of dramatically changed subcluster 4 of macrophages. One-way ANOVA was used for statistical analysis. *** p<. 001, ns=p>0.05.

FIG. 8A-8H show proportion of various cell types and sub-clusters. FIG. 8A display bar-views showing the proportion of neutrophils in whole BMMCs (left). UMAP plot of neutrophils subclusters. FIG. 8B shows myelocytes. FIG. 8C shows myeloblasts. FIG. 8D shows monoblasts. FIG. 8E shows monocytes. FIG. 8F shows DCs. FIG. 8G shows NK/T. FIG. 8H shows Pro-B. One-way ANOVA was used for statistical analysis. * p<. 05, ** p<. 01, *** p<. 001, ns=p>0.05.

FIG. 9A-9D show absence of toxicity with administration of CD24-CAR-T cells. FIG. 9A shows Hematoxylin and eosin (H&E) stained spleen, liver, and kidney samples from normal C57BL/KaLwRij mice. FIG. 9B shows H&E stained spleen, liver, and kidney samples from MM-bearing mice treated with CD24-CAR-T cells. FIG. 9C shows H&E stained spleen, liver, and kidney samples from MM-bearing mice treated with PBS. FIG. 9D shows H&E stained spleen, liver, and kidney samples from MM-burden mice treated with MOCK-CAR-T cells.

FIG. 10A-10I show bispecific CAR-T cells had increased cell killing and macrophage phagocytic clearance in vitro. FIG. 10A shows dual BCMA-CD24-CAR (Bi-CAR), Tandem CD24-BCMA-CAR (Tan1-CAR), and Tandem BCMA-CD24-CAR (Tan2-CAR) constructs. The dual BCMA-CD24-CAR vector with 2 complete CAR units: BCMA-CAR and CD24-CAR. P2A was inserted between these 2 CAR vectors. Two tandem CAR vectors were constructed by CD24-scFv and BCMA-scFv, and 2 scFvs were linked with a (G4S) 4 linker. The safety switch RQR8 was integrated into the hinge regions. FIG. 10B shows the phenotype of CAR-T cells, including the ratio of CD4 and CD8 phenotypes and the ratio of different memory phenotypes. FIG. 10C shows bispecific CAR-T cells cytolytic activity in vitro. CAR-T or T cells were added at the effector/target (E/T) ratio from 1:5 to 5:1. After 24 hours of coculture, cytolytic activity was measured. FIG. 10D shows the expression of T cell activation marker CD69 was detected when the E/T ratio was 5:1 after 24 hours of coculture. FIG. 10E shows IFN-γ concentrations on supernatants were detected when the E/T ratio was 5:1 after 24 hours of coculture. FIG. 10F shows IL-2 concentrations on supernatants were detected when the E/T ratio was 5:1 after 24 hours of coculture. FIG. 10G shows sixteen patient samples with Bi-CAR-T cells or PBS treatment for 24 hours. The percentage of the subpopulation of CD138⁺ cells decreased in 16 of 16 primary myeloma samples post-CART treatment. FIG. 10H shows phagocytosis was performed by coculture of OPM2 cells that expressed GFP (green), DiD-stained macrophages (red), and CAR-T cells at a ratio of 2:1:1. After a 4-hour coculture, suspended cells were washed and detected. Fluorescent images of phagocytic clearance. FIG. 10I is a bar plot showing the percentage of phagocytosis detected by flow cytometry analysis. One-way ANOVA was used in FIG. 10C, FIG. 10D, FIG. 10E, FIG. 10F, FIG. 10I and paired t-test was used in (g). * p<. 05, ** p<. 01, *** p<. 001, ns=p>0.05.

FIG. 11 shows bispecific CAR-T cells release TNF-α in vitro. TNF-α concentrations on supernatants were detected when the E/T ratio was 5:1 after 24 hours of coculture. One-way ANOVA was used for statistical analysis. * p<05, *** p<. 001, ns=p>0.05.

FIG. 12A-12F show bispecific CAR-T cells eliminate MM cells in vivo. FIG. 12A shows bioluminescence images of MM1.S MM-bearing mice treated with CAR-T cells or PBS. FIG. 12B shows quantitative analysis of bioluminescence signals of FIG. 12A. FIG. 12C shows Kaplan-Meier survival analysis of the experiment shown in FIG. 12A. FIG. 12D shows SPE of patient-derived xenograft models. The M-spike is indicated in red. FIG. 12E shows the percentage of the subpopulation of human CD138⁺ cells in BM (top). Representative examples of flow cytometry analysis (bottom). FIG. 12F shows Kaplan-Meier survival analysis of patient-derived xenograft models. One-way ANOVA was used for statistical analysis. * p<. 01, *p<. 001, ns=p>0.05.

FIG. 13A-13D shows bispecific CAR-T cells eliminate MM cells in vivo. FIG. 13A is a schematic of the experimental setup. NSG mice were administered 1.5×10⁶ MM1.S or OPM2 cells by intravenous injection. On day 7 after MM-cell injection, 1.5×10⁶ CAR-T cells were administered. Mice were weighed and monitored for signs of distress every 3 days. Myeloma progression was monitored every 7 days until the mice developed hind-limb paralysis. FIG. 13B shows representative bioluminescence images of OPM2 MM-bearing mice treated with CAR-T cells or PBS. FIG. 13C shows quantitative analysis of bioluminescence signals of FIG. 13B. FIG. 13D shows Kaplan-Meier survival analysis of the experiment shown in FIG. 13B. One-way ANOVA was used for statistical analysis. * p<05, *p<01, *p<. 001.

FIG. 14 illustrates a schematic representation of the roles of the BCMA-CD24-CAR-T cells in MM microenvironment. MM cells expressing CD24 exhibit features of tumor-initiating cells (TICs). In MM microenvironment, CD24 is an important checkpoint molecule for controlling the innate immune response. When CD24 on tumor cells combines with Siglec-10 on macrophages, it causes ITIM region to be phosphorylated, thus blocking Toll-like receptor-mediated inflammation and activating a series of intracellular signal pathways to achieve effective immunosuppression, promoting tumor immune escape and inhibiting cytoskeletal rearrangement, which blocks the macrophage phagocytic clearance. Additionally, MM cells produce chemokines, such as CXCR4 and CXCL12 that promote macrophage migration to the tumor niche and polarize macrophages toward an M2-like phenotype. BCMA-CD24 CAR-T cells could block the CD24-Siglec-10 pathway and promote macrophage phagocytic clearance. Inhibition of the CD24-Siglec-10 pathway also leads to the activation of inflammatory signaling and antitumoral signaling. The IFN-γ and TNFα released by activated BCMA-CD24 CAR-T cells can also promote macrophages polarized to M1Φ and reverse the immunosuppression caused by excessive M20 in the MM microenvironment.

FIG. 15 is a schematic of an exemplary construct of CAR comprising an anti-CD24 antigen recognition domain.

### DETAILED DESCRIPTION

The present disclosure is based, in part, on the surprising discovery that CD24 is expressed in relapsed/refractory malignant B cell or plasma cells. Thus, the present disclosure provides chimeric antigen receptor (CAR) constructs incorporating anti-CD24 antigen binding proteins. Further provided herein are immune effector cells engineered to express CD24-CAR (CD24-CAR immune effector cells) which can block the CD24-Siglec-10 pathway inducing tumor cell clearance by phagocytic macrophages. Inhibition of the CD24-Siglec-10 pathway by the disclosed CD24-CAR immune effector cells can also lead to the activation of inflammatory signaling and anti-tumor signaling. The disclosure further relates to compositions and methods for treating cancer including but not limited to B cell and plasma cell malignancies.

Other aspects and iterations of the invention are described more thoroughly below.

### I. DEFINITIONS

So that the present disclosure may be more readily understood, certain terms are first defined. Unless defined otherwise, all technical and scientific terms used herein have the same meaning as commonly understood by one of ordinary skill in the art to which aspects of the disclosure pertain. Many methods and materials similar, modified, or equivalent to those described herein can be used in the practice of the various aspects of the present disclosure without undue experimentation, the preferred materials and methods are described herein. In describing and claiming the aspects of the present disclosure, the following terminology will be used in accordance with the definitions set out below.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein “CD24” or “cluster of differentiation 24” or “heat stable antigen CD24 (HSA)” refers to peptides derived from the gene CD24 which is located on human chromosome 6 (6q21) (chr6: 106,969,831-106,975,627 Size: 5,797 bases).

CD24 is a cell adhesion molecule. CD24 is rarely expressed in normal human tissue and is predominantly found on B cells, neutrophils, and eosinophils. CD24+ cells upregulate C—X—C chemokine receptor type 4 (CXCR4) expression. The CXCR4/C—X—C chemokine ligand 12 (CXCL12) axis promotes macrophage polarization towards the M2ct phenotype. The M2ct phenotype is immunosuppressive and is associated with shorter survival period of subjects with B cell or plasma cell malignancy, while M1ct are involved in the clearance of tumor cells, and is associated with longer survival period of subjects with B cell or plasma cell malignancy. The CD24/Siglec-10 initiates a signaling cascade that is mediated by SHP-1 and/or SHP-2 phosphatases associated with the two Immunoreceptor Tyrosine-based Inhibition Motifs (ITIMs) in the tail of Siglec-10. These ITIMs inhibit inflammation and cytoskeletal rearrangement ultimately leading to immunosuppression and antiphagocytosis.

Full-length CD24 has an amino acid sequence of SEQ ID NO: 18.

| | |
|---|---|
| SEQ ID | MGRAMVARLGLGLLLLALLLPTQIYSSETTTGTSSNSSQST |
| NO: 18 | SNSGLAPNPTNATTKAAGGALQSTASLFVVSLSLLHLYS |

As used herein “isoform”, refers to any of several different forms of the same protein, arising due to alternative splicing of mRNA encoding the protein, post-translational modification of the protein, proteolytic processing of the protein that occurs in vivo, genetic variations and somatic recombination. The terms “isoform,” “species,” and “variant” are used interchangeably (e.g., the term “CD24 isoform” and “CD24 species” may be used interchangeably).

Unless expressly stated otherwise, the term “CD24” refers to “human CD24”, and encompasses all genetically encoded isoforms or variants, as well as species thereof that are C-terminally truncated in vivo, N-terminally truncated in vivo, N-terminally truncated and C-terminally truncated in vivo, post-translationally modified in vivo, or any combination thereof.

As used herein, “individual”, “subject”, “host”, and “patient” can be used interchangeably and may refer to any human or non-human mammalian subject for whom diagnosis, treatment, prophylaxis or therapy is desired, for example, humans, pets, livestock, horses or other animals. In some aspects, the subject is a human. In other aspects, the subject is a human in need of treatment for cancer.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition, or disorder or those in which the disease, condition or disorder is to be prevented.

As used herein “cancer,” “tumor,” or “malignancy” may refer to one or more neoplasm or cancer. The neoplasm may be malignant or benign, the cancer may be primary or metastatic; the neoplasm or cancer may be early stage or late stage. Non-limiting examples of neoplasms or cancers may include acute lymphoblastic leukemia, acute myeloid leukemia, adrenocortical carcinoma, AIDS-related cancers, AIDS-related lymphoma, anal cancer, appendix cancer, astrocytoma (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

As used herein, treatment of cancer can comprise increased inhibition of cancer progression and/or metastases, inhibition of an increase in tumor volume, a reduction in tumor volume and/or growth, a reduction in tumor growth rate, an eradication or killing of a tumor and/or cancer cell, or any combination thereof. In some aspects, the treatment can also prolong the survival of a subject, improve the prognosis and/or improve the quality of life of the subject.

As used herein “control sample” or “control cell” can be procured from a healthy subject and/or a subject with cancer procured prior to the start of treatment (baseline). A control subject is a healthy subject, or a subject not receiving treatment. In some aspects, the parameters measured during treatment can be an average of several control subjects, or a population average. In some aspects, the control sample can comprise of non-cancer cells. In some aspects, the non-cancer cells can be from the same tissue type as the cancer cells. For example, if the cancer cells are from breast cancer, then the non-cancer cells can be from healthy breast tissue. In some aspects, the control can comprise of an average levels of the analyte in a sample from a subject before onset of cancer. In some aspects, control sample can be a sample from the subject prior to diagnosis or treatment. In certain aspects, the analyte can be measured in a person or persons other than the subject with cancer. In some aspects, the control a person or persons with similar characteristics to the subject with cancer. In some aspects, the control can be an average of the combination of disclosed analyte levels from different healthy sources (e.g., more than one healthy control subject and/or more than one subject prior to the start of treatment (baseline)). In some aspects, the control sample can be pooled sample.

As used herein, a biological sample may be of any biological tissue, fluid, or cell from the subject. The sample can be solid or fluid. The sample can be a heterogeneous cell population. Non-limiting examples of suitable biological samples include sputum, serum, blood, blood cells (e.g., white cells), a biopsy, urine, peritoneal fluid, pleural fluid, or cells derived therefrom. The biopsy can be a fine needle aspirate biopsy, a core needle biopsy, a vacuum assisted biopsy, an open surgical biopsy, a shave biopsy, a punch biopsy, an incisional biopsy, a curettage biopsy, or a deep shave biopsy. Biological samples may also include sections of tissues, such as frozen sections or formalin fixed sections taken for histological purposes. A sample can be a tumor tissue, tissue surrounding a tumor, or non-tumor tissue. Methods of procuring a biological sample from a subject are well known in the art.

As used herein “antibody” is used in the broadest sense and encompasses various antibody and antibody-like structures, including but not limited to full-length monoclonal, polyclonal, and multispecific (e.g., bispecific, trispecific, etc.) antibodies, as well as heavy chain antibodies and antibody fragments provided they exhibit the desired antigen-binding activity. The domain(s) of an antibody that is involved in binding an antigen is referred to as a “variable region” or “variable domain,” and is described in further detail below. A single variable domain may be sufficient to confer antigen-binding specificity. Preferably, but not necessarily, antibodies useful in the discovery are produced recombinantly. Antibodies may or may not be glycosylated, though glycosylated antibodies may be preferred. An “isolated” antibody is one which has been separated from a component of its natural environment. In some aspects, an antibody is purified to greater than 95% or 99% purity as determined by methods known in the art.

As used herein “full length antibody” and “intact antibody” are interchangeable, and refer to an antibody having a structure substantially similar to a native antibody structure or having heavy chains that contain an Fc region as defined herein. The basic structural unit of a native antibody comprises a tetramer. Each tetramer is composed of two identical pairs of polypeptide chains, each pair having one “light” chain (about 25 kDa) and one “heavy” chain (about 50-70 kDa). Light chains are classified as gamma, mu, alpha, and lambda. Heavy chains are classified as gamma, mu, alpha, delta, or epsilon, and define the antibody's isotype as IgG, IgM, IgA, IgD and IgE, respectively. The amino-terminal portion of each light and heavy chain includes a variable region of about 100 to 110 or more amino acid sequences primarily responsible for antigen recognition (VL and VH, respectively). The carboxy-terminal portion of each chain defines a constant region primarily responsible for effector function. Within light and heavy chains, the variable and constant regions are joined by a “J” region of about 12 or more amino acid sequences, with the heavy chain also including a “D” region of about 10 more amino acid sequences. Intact antibodies are properly cross-linked via disulfide bonds, as is known in the art.

The variable domains of the heavy chain and light chain of an antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

As used herein “Framework region” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence: FR1-HVR1-FR2-HVR2-FR3-HVR3-FR4. The FR domains of a heavy chain and a light chain may differ, as is known in the art.

As used herein “hypervariable region” or “HVR” refers to each of the regions of a variable domain which are hypervariable in sequence (also commonly referred to as “complementarity determining regions” or “CDR”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). As used herein, “an HVR derived from a variable region” refers to an HVR that has no more than two amino acid substitutions, as compared to the corresponding HVR from the original variable region. Exemplary HVRs herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); (d) CDR1-IMGT (positions 27-38), CDR2-IMGT (positions 56-65), and CDR3-IMGT regions (positions 105-116 or 105-117), which are based on IMGT unique numbering (Lefranc, “The IMGT unique numbering for Immunoglobulins, T cell receptors and Ig-like domains,” The Immunologist, 1999, 7:132-136; Lefranc et al., Nucleic Acids Research, 2009, 37 (Database issue): D1006-D1012; Ehrenmann et al., “Chapter 2: Standardized Sequence and Structure Analysis of Antibody Using IMGT,” in Antibody Engineering Volume 2, Eds. Roland E. Kontermann and Stefan Dubel, 2010, Springer-Verlag Berlin Heidelberg, doi: 10.1007/978-3-642-01147-4; www.imgt.org/IMGTScientificChart/Nomenclature/IMGT-FRCDRdefinition.html), and (e) combinations of (a), (b), (c), and/or (d), as defined below for various antibodies of this disclosure. Unless otherwise indicated, HVR residues and other residues in the variable domain (e.g., FR residues) that are assigned sequence identification numbers are numbered based on IMGT unique numbering, supra.

As used herein “Fc region” define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5^{th} Ed. Public Health Service, National Institutes of Health, Bethesda, Md., 1991.

A “variant Fc region” comprises an amino acid sequence that can differ from that of a native Fc region by virtue of one or more amino acid substitution(s) and/or by virtue of a modified glycosylation pattern, as compared to a native Fc region or to the Fc region of a parent polypeptide. In an example, a variant Fc region can have from about one to about ten amino acid substitutions, or from about one to about five amino acid substitutions in a native sequence Fc region or in the Fc region of the parent polypeptide. The variant Fc region herein may possess at least about 80% homology, at least about 90% homology, or at least about 95% homology with a native sequence Fc region and/or with an Fc region of a parent polypeptide.

As used herein “antibody fragment” refers to a molecule other than an intact antibody that comprises a portion of an intact antibody that binds the antigen to which the intact antibody binds. Non-limiting examples of antibody fragments include but are not limited to Fv, Fab, Fab′, Fab′-SH, F(ab′)₂; single-chain forms of antibodies and higher order variants thereof; single-domain antibodies, and multispecific antibodies formed from antibody fragments.

Single-chain forms of antibodies, and their higher order forms, may include, but are not limited to, single-domain antibodies, single chain variant fragments (scFvs), divalent scFvs (di-scFvs), trivalent scFvs (tri-scFvs), tetravalent scFvs (tetra-scFvs), diabodies, and triabodies and tetrabodies. ScFv's are comprised of heavy and light chain variable regions connected by a linker. In most instances, but not all, the linker may be a peptide. A linker peptide is preferably from about 5 to 30 amino acids in length, or from about 10 to 25 amino acids in length. Typically, the linker allows for stabilization of the variable domains without interfering with the proper folding and creation of an active binding site. In some aspects, a linker peptide is rich in glycine, as well as serine or threonine. ScFvs can be used to facilitate phage display or can be used for flow cytometry, immunohistochemistry, or as targeting domains. Methods of making and using scFvs are known in the art. ScFvs may also be conjugated to a human constant domain (e.g. a heavy constant domain is derived from an IgG domain, such as IgG1, IgG2, IgG3, or IgG4, or a heavy chain constant domain derived from IgA, IgM, or IgE). Diabodies, triabodies, and tetrabodies and higher order variants are typically created by varying the length of the linker peptide from zero to several amino acids. Alternatively, it is also well known in the art that multivalent binding antibody variants can be generated using self-assembling units linked to the variable domain.

As used herein “single-domain antibody” refers to an antibody fragment consisting of a single, monomeric variable antibody domain.

As used herein “Multispecific antibodies” include bi-specific antibodies, tri-specific, or antibodies of four or more specificities. Multispecific antibodies may be created by combining the heavy and light chains of one antibody with the heavy and light chains of one or more other antibodies. These chains can be covalently linked.

As used herein “Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

As used herein “heavy chain antibody” refers to an antibody that consists of two heavy chains. A heavy chain antibody may be an IgG-like antibody from camels, llamas, alpacas, sharks, etc., or an IgNAR from a cartilaginous fish.

As used herein “humanized antibody” refers to a non-human antibody that has been modified to reduce the risk of the non-human antibody eliciting an immune response in humans following administration but retains similar binding specificity and affinity as the starting non-human antibody. A humanized antibody binds to the same or similar epitope as the non-human antibody. The term “humanized antibody” includes an antibody that is composed partially or fully of amino acid sequences derived from a human antibody germline by altering the sequence of an antibody having non-human hypervariable regions (“HVR”). The simplest such alteration may consist simply of substituting the constant region of a human antibody for the murine constant region, thus resulting in a human/murine chimera which may have sufficiently low immunogenicity to be acceptable for pharmaceutical use. Preferably, the variable region of the antibody is also humanized by techniques that are by now well known in the art. For example, the framework regions of a variable region can be substituted by the corresponding human framework regions, while retaining one, several, or all six non-human HVRs. Some framework residues can be substituted with corresponding residues from a non-human VL domain or VH domain (e.g., the non-human antibody from which the HVR residues are derived), e.g., to restore or improve specificity or affinity of the humanized antibody. Substantially human framework regions have at least about 75% homology with a known human framework sequence (i.e. at least about 75%, at least about 80%, at least about 85%, at least about 90%, at least about 95%, or at least about 99% sequence identity). HVRs may also be randomly mutated such that binding activity and affinity for the antigen is maintained or enhanced in the context of fully human germline framework regions or framework regions that are substantially human. As mentioned above, it is sufficient for use in the methods of this discovery to employ an antibody fragment. Further, as used herein, the term “humanized antibody” refers to an antibody comprising a substantially human framework region, at least one HVR from a nonhuman antibody, and in which any constant region present is substantially human. Substantially human constant regions have at least about 90% with a known human constant sequence (i.e. about 90%, about 95%, or about 99% sequence identity). Hence, all parts of a humanized antibody, except possibly the HVRs, are substantially identical to corresponding pairs of one or more germline human immunoglobulin sequences.

If desired, the design of humanized immunoglobulins may be carried out as follows, or using similar methods familiar to those with skill in the art. A murine antibody variable region is aligned to the most similar human germline sequences (e.g. by using BLAST or similar algorithm). The CDR residues from the murine antibody sequence are grafted into the similar human “acceptor” germline. Subsequently, one or more positions near the CDRs or within the framework (e.g., Vernier positions) may be reverted to the original murine amino acid in order to achieve a humanized antibody with similar binding affinity to the original murine antibody. Typically, several versions of humanized antibodies with different reversion mutations are generated and empirically tested for activity. The humanized antibody variant with properties most similar to the parent murine antibody and the fewest murine framework reversions is selected as the final humanized antibody candidate.

As used herein, the term “fusion protein” refers to proteins created through the joining of two or more genes that originally coded for separate proteins. Translation of this fusion gene results in a single polypeptide or multiple polypeptides with functional properties derived from each of the original proteins. In some embodiments, the two or more genes may comprise a substitution, a deletion, and/or an addition in its nucleotide sequence.

As used herein “expression” or “expression level” or “level of expression” refers to amount of a particular analyte (e.g., CD24) present in the sample. The amount may be a concentration, number, ratio, proportion, or a percentage of the analyte compared to the control sample or determined using a standard curve. The amount may be an absolute amount or a relative amount.

As used herein “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “composition” or “pharmaceutical composition” as used herein refers to an immunotherapeutic cell population in combination with one or more therapeutically acceptable carriers.

The term “deglycosylation” as used herein refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “dimer” is an oligomer consisting of two monomers joined by bonds that can be either strong or weak, covalent, or intermolecular. The term “homodimer” is used when the two molecules are identical, e.g. A-A, and “heterodimer” when they are not, e.g. A-B.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “effector function” refers to a specialized function of a differentiated cell. An effector function of a T cell, for example, may be cytolytic activity or helper activity including the secretion of cytokines. An effector function in a naive, memory, or memory-type T cell may also include antigen-dependent proliferation.

The term “fratricide” as used herein means a process which occurs when a CAR-T cell becomes the target of, and is killed by, another CAR-T cell comprising the same chimeric antigen receptor as the target of CAR-T cell, because the targeted cell expresses the antigen specifically recognized by the chimeric antigen receptor on both cells. CAR-T cells comprising a chimeric antigen receptor which are deficient in an antigen to which the chimeric antigen receptor specifically binds will be “fratricide-resistant.”

As used herein, the term “gene expression” or “expression” of a protein is understood to refer to transcription of a DNA sequence, translation of an mRNA transcript, and secretion of a fusion protein product, or an antibody, or an antibody fragment thereof.

The term “genome-edited” as used herein means having a gene added, deleted, or modified to be non-functional. Thus, in certain aspects, a “gene-edited T cell” or a “modified T cell” is a T cell that has had a gene such as a CAR recognizing at least one antigen added; and/or has had a gene such as the gene(s) to the antigen(s) that are recognized by the CAR deleted.

A “healthy donor,” as used herein, is one who does not have a malignancy (e.g., a B cell or plasma cell malignancy).

As used herein, the term “host cell” refers to a prokaryotic cell and/or a eukaryotic cell into which a recombinant expression vector can be introduced.

Unless otherwise specified, the terms “protein”, “polypeptide”, and “peptide” may be used as an interchangeable concept.

The term “immune checkpoint inhibitor” refers to a type of drug that blocks certain proteins made by some types of immune system cells, such as T cells, and some cancer cells.

The term “immune effector cell,” as used herein, are cells that are actively involved in the destruction of tumor cells, e.g., possess anti-tumor activity. These cells may include, but are not limited to, natural killer (NK) cells, cytotoxic T cells, and memory T cells.

As used herein “Chimeric antigen receptor” or “CAR”, as used herein, refers to a recombinant fusion protein that has an extracellular domain, i.e. an antigen recognition domain or target element, coupled to an intracellular domain comprising co-stimulatory and signaling domains, which directs the cell to perform a specialized function upon binding of an antigen. The terms “artificial T cell receptor,” “chimeric T-cell receptor,” and “chimeric immunoreceptor” may each be used interchangeably herein with the term “chimeric antigen receptor.” Chimeric antigen receptors are distinguished from other antigen binding agents by their ability to both bind MHC-independent antigen and transduce activation signals via the components of the intracellular domain.

In some aspects, the CARs of the invention comprise at least two antigen-specific targeting regions, an extracellular domain, a transmembrane domain, one or more co-stimulatory domains, and an intracellular signaling domain. The two or more antigen-specific targeting regions target at least two different antigens and may be arranged in tandem and separated by linker sequences. In aspects, the extracellular spacer domain is optional. In another aspect, the CAR is a bispecific CAR. A bispecific CAR is specific to two different antigens.

As used herein “Co-stimulatory domain” or “CSD” refers to the portion of the CAR which enhances the proliferation, survival and/or development of memory cells. The CARs of the disclosure may comprise one or more co-stimulatory domains.

As used herein “Extracellular spacer domain” or “ESD” refers to the hydrophilic region which is between the antigen-specific targeting region and the transmembrane domain. In some aspects, the CARs of the disclosure comprise an extracellular spacer domain. In other aspects, the CARs of the invention do not comprise an extracellular spacer domain. Exemplary extracellular spacer domain include but are not limited to CD8a hinge.

As used herein “Intracellular signaling domain” or “ISD” or “cytoplasmic domain” refer to the portion of the CAR which transduces the effector function signal and directs the cell to perform its specialized function.

As used herein “Transmembrane domain” or “TMD” refers to the region of the CAR which crosses the plasma membrane.

The term “chimeric antigen receptor (CAR)-bearing immune effector cells” or “chimeric antigen receptor (CAR) modified immune effector cells” are immune effector cells that express a chimeric antigen receptor. These cells may include, but are not limited to, CAR-T cells or CAR-bearing NK cells.

The term “CAR-T cell” means a CAR-T cell that expresses a chimeric antigen receptor.

A dual CAR-T cell (equivalently, dCAR-T) is a CAR-T cell that expresses two distinct chimeric antigen receptor polypeptides with affinity to different target antigens expressed within the same effector cell or separate epitopes within the same target protein, wherein each CAR functions independently. The CAR may be expressed from a single polynucleotide sequence or multiple polynucleotide sequences.

A tandem CAR-T cell (equivalently, tCAR-T) is a CAR-T cell with a single chimeric antigen polypeptide containing two distinct antigen recognition domains with affinity to different targets or separate epitopes within the same target protein, wherein the antigen recognition domains are linked through a peptide linker and share common costimulatory domain(s), and wherein binding of either antigen recognition domain will signal though a common costimulatory domains(s) and signaling domain.

The term “patient” is generally synonymous with the term “subject” and includes all mammals including humans.

As used herein, the term “signal sequence,” or equivalently, “signal peptide,” refers to a fragment directing the secretion of a biologically active molecule drug and a fusion protein, and it is cut off after being translated in a host cell. The signal sequence as used herein is a polynucleotide encoding an amino acid sequence initiating the movement of the protein penetrating the endoplasmic reticulum (ER) membrane. Useful signal sequences include an antibody light chain signal sequence, e.g., antibody 14.18 (Gillies et al . . . , J. Immunol. Meth 1989. 125:191-202), an antibody heavy chain signal sequence, e.g., MOPC141 an antibody heavy chain signal sequence (Sakano et al., Nature, 1980.286:676-683), and other signal sequences know in the art (e.g., see Watson et al., Nucleic Acid Research, 1984.12:5145-5164). The characteristics of signal peptides are well known in the art, and the signal peptides conventionally having 16 to 30 amino acids, but they may include more or less number of amino acid residues. Conventional signal peptides consist of three regions of the basic N-terminal region, a central hydrophobic region, and a more polar C-terminal region.

The term “therapeutically acceptable” refers to substances which are suitable for use in contact with the tissues of patients without undue toxicity, irritation, and allergic response, are commensurate with a reasonable benefit/risk ratio, and/or are effective for their intended use.

The term “therapeutically effective” or “effective” amount is intended to qualify the amount of active ingredients used in the treatment of a disease or disorder or on the effecting of a clinical endpoint.

As used herein, the terms “transduced”, “transformed”, and “transfected” refer to the introduction of a nucleic acid (e.g., a vector) into a cell using a technology known in the art.

As used herein, the term “vector” is understood as a nucleic acid means which includes a nucleotide sequence that can be introduced into a host cell to be recombined and inserted into the genome of the host cell, or spontaneously replicated as an episome. The vector may include linear nucleic acids, plasmids, phagemids, cosmids, RNA vectors, virus vectors, and analogs thereof. Examples of the virus vectors may include retroviruses, adenoviruses, and adeno-associated viruses, but are not limited thereto.

As used herein “nucleic acid”, “nucleic acid molecule”, and “polynucleotide” are used interchangeably herein. The terms “nucleic acid encoding . . . ”, or “nucleic acid molecule encoding . . . “, should be understood as referring to the sequence of nucleotides which encodes a polypeptide.

A polynucleotide described herein may comprise one or more nucleic acids each encoding a polypeptide, all operably linked to (i.e., in a functional relationship with) one or more regulatory sequences, such as a promoter. Such a polynucleotide may alternatively be referred to herein as a “nucleic acid construct” or “construct”.

As used herein “polypeptide” and “protein” are used interchangeably to refer to a polymer of amino acid residues.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length (in amino acids or nucleotides) of two given SEQ ID NOS or based on a portion thereof. A portion of a full-length sequence may be referred to as a fragment, and preferably means at least 50%, 60%, 70%, 80%, 90%, or 100% of the length (in amino acids or nucleotides) of a reference sequence. “Identity” also refers to the degree of sequence relatedness between two amino acid sequences, or between two nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of this sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined, for example, by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below:

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, |
| | V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in | A, C, D, E, G, H, K, N, Q, R, |
| turn formation | S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gin; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

As used herein “binds”, refers to a polypeptide (including antibodies) or receptor, binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an antigen) when it does not bind in a significant amount to other proteins present in the sample or to other proteins to which the ligand or antibody may come in contact in an organism.

A “genetically modified” or “modified cell” refers to a cell in which the nuclear, organellar or extrachromosomal nucleic acid sequences of a cell has been transformed, modified or transduced using recombinant DNA technology to comprise a heterologous nucleic acid molecule, and is used interchangeably with “engineered cell,” “transformed cell,” and “transduced cell.” In one aspect, a genetically modified cell as disclosed herein expresses a protein encoded by a nucleic acid molecule engineered in such manner to contain an insertion of at least one nucleotide, a deletion of at least one nucleotide, and/or a substitution of at least one nucleotide in a sequence encoding at least one heterologous protein. “Engineered cells” refers herein to cells having been engineered, e.g., by the introduction of an exogenous nucleic acid sequence as defined herein. Such a cell has been genetically modified for example by the introduction of for example one or more mutations, insertions and/or deletions in the endogenous gene and/or insertion of a genetic construct in the genome. The modification may have been introduced using recombinant DNA technology. An engineered cell may refer to a cell in isolation or in culture. Engineered cells may be “transduced cells” wherein the cells have been infected with e.g., a modified virus, for example, a retrovirus may be used but other suitable viruses may also be contemplated such as lentiviruses. Non-viral methods may also be used, such as transfections. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient but may also be a stable transfection wherein cells can be selected that have the gene construct integrated in their genome. In some cases, genetic engineering systems such as CRISPR or Argonaute may be utilized to design engineered cells that express a polypeptide described herein.

As used herein “Linker” or “linker domain” or “linker region” refer to an oligo- or polypeptide region from about 1 to 100 amino acids in length, which links together any of the domains/regions of the CAR of the invention. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. In other aspects, the linker sequences may comprise Asp-Val/11e-Glu-X-Asn-Pro-Gly pro motif (SEQ ID NO: 19), which results in cleavage between the glycine and the second proline. Other linkers will be apparent to those of skill in the art and may be used in connection with alternate aspects of the invention.

### II. CAR Expressing Cells

The disclosure provides chimeric antigen receptor (CAR), immune effector cells that express a chimeric antigen receptor, compositions, methods of using the same.

A chimeric antigen receptor (CAR) polypeptide includes a signal peptide, an antigen recognition domain, a hinge region, a transmembrane domain, at least one co-stimulatory domain, and a signaling domain. The hinge region and the antigen recognition domain may be collectively referred to as the extracellular domain. The at least one co-stimulatory domain and signaling domain may be collectively referred to as the intracellular domain. The extracellular and intracellular domains of a CAR are discussed in more detail below.

First-generation CARs include CD35 as a signaling domain, whereas second-generation CARs include at least one single co-stimulatory domain, which can be derived from various proteins. Examples of co-stimulatory domains include, but are not limited to, CD28, CD2, 4-1BB (CD137), and OX-40 (CD134). Third generation CARs include two co-stimulatory domains selected from, but not limited to, CD28, 4-1BB (CD137), OX-40 (CD134), and CD2.

In some aspects, the CAR of the present disclosure comprises a CD35 as a signaling domain. In some aspects, the CD35 signaling domain of the disclosed CAR comprises the nucleic acid sequence:

(SEQ ID NO: 1)
AGGGTGAAGTTCAGCAGGAGCGCCGACGCCCCCGCCTACCAGCAG
GGCCAGAACCAGCTGTACAACGAGCTGAACCTGGGCAGGAGGGAG
GAGTACGACGTGCTGGACAAGAGGAGGGGCAGGGACCCCGAGATG
GGCGGCAAGCCCCAGAGGAGGAAGAACCCCCAGGAGGGCCTGTAC
AACGAGCTGCAGAAGGACAAGATGGCCGAGGCCTACAGCGAGATC
GGCATGAAGGGCGAGAGGAGGAGGGGCAAGGGCCACGACGGCCTG
TACCAGGGCCTGAGCACCGCCACCAAGGACACCTACGACGCCCTG
CACATGCAGGCCCTGCCCCCCAGG.

In some aspects, the signaling domain of a chimeric antigen receptor of the disclosure comprises a CD35, comprising nucleic acid sequence of SEQ ID NO: 1, wherein the said nucleic acid sequence comprises at least 60% sequence identity or similarity with nucleic acid sequence SEQ ID NO: 1. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects the CD35 signaling domain of the disclosed CAR comprises the amino acid sequence:

(SEQ ID NO: 2)
RVKFSRSADAPAYQQGQNQLYNELNLGRREEYDVLDKRRGRDPEMG
GKPQRRKNPQEGLYNELQKDKMAEAYSEIGMKGERRRGKGHDGLYQ
GLSTATKDTYDALHMQALPPR.

In some aspects, the signaling domain of a chimeric antigen receptor of the disclosure comprises a CD35, comprising amino acid sequence of SEQ ID NO: 2, wherein the said amino acid sequence comprises at least 60% sequence identity or similarity with amino acid sequence SEQ ID NO: 2. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the CAR of the present disclosure comprises 4-1BB as a co-stimulatory domain. In some aspects, 4-1BB co-stimulatory domain of the disclosed CAR comprise the nucleic acid sequence:

(SEQ ID NO: 3)
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG.

In some aspects, the co-stimulatory domain of a chimeric antigen receptor of the disclosure comprises a 4-1BB, comprising nucleic acid sequence of SEQ ID NO: 3, wherein the said nucleic acid sequence comprises at least 60% sequence identity or similarity with nucleic acid sequence SEQ ID NO: 3. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, 4-1BB co-stimulatory domain of the disclosed CAR comprise the amino acid sequence:

(SEQ IN NO: 4)
KRGRKKLLYIFKQPFMRPVQTTQEEDGCSCRFPEEEEGGCEL

In some aspects, the co-stimulatory domain of a chimeric antigen receptor of the disclosure comprises a 4-1BB, comprising amino acid sequence of SEQ ID NO: 4, wherein the said amino acid sequence comprises at least 60% sequence identity or similarity with nucleic acid sequence SEQ ID NO: 4. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

The signal peptide includes a peptide sequence that directs the transport and localization of the peptide and any attached polypeptide within a cell, e.g. to a certain cell organelle (such as the endoplasmic reticulum) and/or the cell surface. The terms “signal peptide” and “leader sequence” are used interchangeably.

The signal peptide directs the transport of any secreted or transmembrane protein to the cell membrane and cell surface allowing correct localization of the polypeptide. In particular, the signal peptide of the present disclosure directs the polypeptide to the cellular membrane, wherein the extracellular portion, i.e. antigen recognition domain or target element of the polypeptide is displayed on the cell surface, the transmembrane portion spans the plasma membrane, and the signaling domain is in the cytoplasmic portion, or interior of the cell.

In some aspects, the signal peptide includes the signal peptide from human CD8a. In some aspects, the signal peptide may be a functional fragment of the CD8a signal peptide. A functional fragment includes a fragment of at least 10 amino acids of the CD8a signal peptide that directs the appended polypeptide to the cell membrane and cell surface.

In some aspects, the CD8a signal domain of the disclosed CAR comprises the nucleic acid sequence:

(SEQ ID NO: 5)
ATGGCCTTACCAGTGACCGCCTTGCTCCTGCCGCTGGCCTTGCTGCTCCA
CGCCGCCAGGCCG.

In some aspects, the signal domain of a chimeric antigen receptor of the disclosure comprises a CD8a, comprising nucleic acid sequence of SEQ ID NO: 5, wherein the said nucleic acid sequence comprises at least 60% sequence identity or similarity with nucleic acid sequence SEQ ID NO: 5. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the CD8a signal domain of the disclosed CAR comprises the amino acid sequence:

(SEQ ID NO: 6)
MALPVTALLLPLALLLHAARP.

In some aspects, the signal domain of a chimeric antigen receptor of the disclosure comprises a CD8a, comprising amino acid sequence of SEQ ID NO: 6, wherein the said amino acid sequence comprises at least 60% sequence identity or similarity with nucleic acid sequence SEQ ID NO: 6. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

The antigen recognition domain or target element of a chimeric antigen receptor includes a polypeptide that is selective for or targets an antigen, receptor, peptide ligand, protein ligand of the target, or a polypeptide of the target.

The antigen recognition domain of a chimeric antigen receptor recognizes and specifically binds an antigen, typically a surface-expressed antigen of a malignancy. An antigen recognition domain specifically binds an antigen when, for example, it binds the antigen with an affinity constant or affinity of interaction (KD) between about 0.1 pM to about 10 pM, preferably about 0.1 pM to about 1 pM, more preferably about 0.1 pM to about 100 nM. Methods for determining the affinity of interaction are well-known in the art. An antigen-specific extracellular domain suitable for use in a CAR of the present disclosure may be any antigen-binding polypeptide, a wide variety of which are well-known in the art. In some instances, the antigen-binding domain is a single chain Fv (scFv). The single-chain variable fragment (scFv) is expressed on the surface of a CAR-immune effector cell and confers antigen specificity. The scFv is derived from the portion of an antibody that specifically recognizes a target protein. Other antibody-based recognition domains (cAb VHH (camelid antibody variable domains) and humanized versions thereof, IgNAR VH (shark antibody variable domains) and humanized versions thereof, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

In some aspects, an antigen specifically bound by the chimeric antigen receptor of a CAR-immune effector cell, and optionally the antigen for which the CAR-immune effector cell is deficient, is an antigen expressed on a malignant cell, more preferably an antigen that is overexpressed on malignant cell in comparison to a non-malignant cell. A “malignant cell” is a cell derived from a cell malignancy. Suitable CAR targeted antigens may include antigens found on the surface of abnormal B cell or plasma cell, for e.g., multiple myeloma cell, i.e., a malignant plasma cell.

For instance, in one aspect, CD24 is a suitable CAR targeted antigen expressed on a malignant cell. In some aspects, a CAR-immune effector cell of the present disclosure comprises an antigen recognition domain of a chimeric antigen receptor that specifically binds to CD24.

In one aspect, an anti-CD24 antigen recognition domain of a chimeric antigen receptor of the disclosure comprises a scFv comprising the nucleic acid sequence:

(SEQ ID NO: 7)
GACGTGCACCTGCAGGAGAGCGGCCCCGACCTGGTGAAGCCCAGCCAGAG
CCTGAGCCTGACCTGCACCGTGACCGGCTACAGCATCACCAGCGGCTACA
CCTGGCACTGGATCAGGCAGTTCCCCGGCAACACCGTGGAGTGGATGGGC
TACATCCAGTACACCGGCAGCACCAGGTACAACCCCGCCCTGAGGGGCAG
GCTGAGCATCAGCAGGGACACCAGCAAGAACCAGTTCTTCCTGCAGCTGA
TCAGCGTGACCACCGCCGACACCGGCACCTACTTCTGCGCCAGGGGCACC
ACCGCCAGCTTCGACTACTGGGGCCAGGGCACCACCCTGACCGTGGCCAG
CGCCGGCGGCGGCGGCAGCGGCGGCGGCGGCAGCGGCGGCGGCGGCAGCG
ACATCGTGATGAGCCAGAGCCCCAGCAGCCTGAACGTGAGCGTGGGCGAG
AAGGTGACCATGAGGTGCAGGAGCAGCCAGAGCCTGCTGTACAGCAGCGA
CCAGAAGAACTACCTGACCTGGTACCAGCAGAAGCCCGGCCAGAGCCCCA
AGCTGCTGATCAGCTGGGCCAGCACCAGGGCCAGCGGCGTGCCCGACAGG
TTCACCGGCAGCGGCAGCGGCACCGACTTCACCCTGACCATCAGCAGCGT
GAAGGCCGAGGACCTGGGCGTGTACTACTGCCAGCAGTACTTCATCTACC
CCCTGACCTTCGGCGTGGGCACCAAGCTGGGCCTGAAG.

In some aspects, an anti-CD24 antigen recognition domain of a chimeric antigen receptor of the disclosure comprises a scFv, comprising nucleic acid sequence of SEQ ID NO: 7, wherein the said nucleic acid sequence comprises at least 60% sequence identity or similarity with nucleic acid sequence SEQ ID NO: 7. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, disclosed CAR comprises an anti-CD24 antigen recognition domain comprising a scFv with at least about 80% (e.g., about 85%, about 90%, about 95%, about 98%) identity to the sequence of SEQ ID NO: 7.

In some aspects, disclosed CAR comprises an anti-CD24 antigen recognition domain comprising a scFv with 100% identity to the sequence of SEQ ID NO: 7.

In one aspect, an anti-CD24 antigen recognition domain of a chimeric antigen receptor of the disclosure comprises a scFv comprising the amino acid sequence:

(SEQ ID NO: 8)
DVHLQESGPDLVKPSQSLSLTCTVTGYSITSGYTWHWIRQFPGNTVEWMG
YIQYTGSTRYNPALRGRLSISRDTSKNQFFLQLISVTTADTGTYFCARGT
TASFDYWGQGTTLTVASAGGGGSGGGGSGGGGSDIVMSQSPSSLNVSVGE
KVTMRCRSSQSLLYSSDQKNYLTWYQQKPGQSPKLLISWASTRASGVPDR
FTGSGSGTDFTLTISSVKAEDLGVYYCQQYFIYPLTFGVGTKLGLK

In some aspects, an anti-CD24 antigen recognition domain of a chimeric antigen receptor of the disclosure comprises a scFv, comprising amino acid sequence of SEQ ID NO: 8, wherein the said amino acid sequence comprises at least 60% sequence identity or similarity with amino acid sequence SEQ ID NO: 8. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, disclosed CAR comprises an anti-CD24 antigen recognition domain comprising a scFv with at least about 80% (e.g., about 85%, about 90%, about 95%, about 98%) identity to the sequence of SEQ ID NO: 8.

In some aspects, disclosed CAR comprises an anti-CD24 antigen recognition domain comprising a scFv with 100% identity to the sequence of SEQ ID NO: 8.

The hinge domain is a structure between the antigen recognition domain and the cell plasma membrane; these sequences are generally derived from IgG subclasses (such as IgG1 and IgG4), IgD and CD8 domains, of which IgG1 has been the most extensively used for CAR construction. Currently, studies of the hinge domain mainly focus on the following four aspects: 1) reducing binding affinity to the Fcγ receptor, thereby eliminating off-target activation; 2) enhancing the single-chain variable fragment (scFv) flexibility, thereby relieving the spatial constraints between tumor antigens and CARs, and in turn promoting synapse formation between the CAR-immune effector cells and target cells; 3) reducing the distance between an scFv and the target epitope; and 4) facilitating the detection of CAR expression using anti-Fc reagents.

In one aspect, the hinge domain includes a hinge domain of a human protein selected from CD28, 4-1BB (CD137), OX-40 (CD134), CD35, T cell receptor α or β chain, CD45, CD4, CD5, CD8, CD8x, CD9, CD16, CD22, CD33, CD37, CD64, CD80, CD86, ICOS, CD154, functional derivatives and/or combinations thereof. In some aspects, the hinge domain of a chimeric antigen receptor of the disclosure comprises a CD8.

The transmembrane domain includes a hydrophobic polypeptide that spans the cellular membrane. In particular, the transmembrane domain spans from one side of a cell membrane (extracellular) through to the other side of the cell membrane (intracellular or cytoplasmic).

The transmembrane domain may be in the form of an alpha helix or a beta barrel, or combinations thereof. The transmembrane domain may include a polytopic protein, which has many transmembrane segments, each alpha-helical, beta sheets, or combinations thereof.

In one aspect, the transmembrane domain that is naturally associated with one of the domains in the CAR is used. In another aspect, the transmembrane domain is selected or modified by amino acid substitution to avoid binding of such domains to the transmembrane domains of the same or different surface membrane proteins to minimize interactions with other members of the receptor complex.

For example, the transmembrane domain is selected from a transmembrane domain of a T-cell receptor α or β chain, a CD35 chain, CD28, CD3s, CD45, CD4, CD5, CD7, CD8, CD9, CD16, CD22, CD33, CD37, CD64, CD80, CD86, CD68, OX-40 (CD134), 4-1BB (CD137), ICOS, CD41, CD154, functional derivatives and/or combinations thereof. In some aspects, the transmembrane domain is a CD8.

In some aspects, the hinge and the transmembrane domain of the disclosed CAR comprises the nucleic acid sequence:

(SEQ ID NO: 9)
ACCACGACGCCAGCGCCGCGACCACCAACACCGGCGCCCACCATCGCGTC
GCAGCCCCTGTCCCTGCGCCCAGAGGCGTGCCGGCCAGCGGCGGGGGGCG
CAGTGCACACGAGGGGGCTGGACTTCGCCTGTGATATCTACATCTGGGCG
CCCTTGGCCGGGACTTGTGGGGTCCTTCTCCTGTCACTGGTTATCACCCT
TTACTGC.

In some aspects, the hinge and the transmembrane domain of a chimeric antigen receptor of the disclosure comprises nucleic acid sequence of SEQ ID NO: 9, wherein the said nucleic acid sequence comprises at least 60% sequence identity or similarity with nucleic acid sequence SEQ ID NO: 9. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the hinge and the transmembrane domain of the disclosed CAR comprises the amino acid sequence:

(SEQ ID NO: 10)
TTTPAPRPPTPAPTIASQPLSLRPEACRPAAGGAVHTRGLDFACDIYIWA
PLAGTCGVLLLSLVITLYC.

In some aspects, the hinge and the transmembrane domain of a chimeric antigen receptor of the disclosure comprises amino acid sequence of SEQ ID NO: 10, wherein the said amino acid sequence comprises at least 60% sequence identity or similarity with amino acid sequence SEQ ID NO: 10. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Typically, the extracellular domain, i.e. an antigen recognition domain or target element is linked to the intracellular domain, i.e., the co-stimulatory and signaling domain(s) of the chimeric antigen receptor by a transmembrane domain. A transmembrane domain traverses the cell membrane, anchors the CAR to the immune effector cell surface, and connects the extracellular domain to the intracellular domain, thus impacting expression of the CAR on the immune effector cell surface.

A chimeric antigen receptor of the present disclosure may comprise one or more costimulatory domain and/or one or more spacers. A costimulatory domain is derived from costimulatory proteins that enhance cytokine production, proliferation, cytotoxicity, and/or persistence in vivo.

In one embodiment, the co-stimulatory domain is selected from OX-40 (CD134), CD27, CD28, CD30, CD40, PD-1, CD2, CD7, CD258, Natural killer Group 2 member C (NKG2C), Natural killer Group 2 member D (NKG2D), B7-H3, a ligand that binds to at least one of CD83, ICAM-1, LFA-1 (CD11a/CD18), ICOS, and 4-1BB (CD137), CDS, ICAM-1, LFA-1 (CDIa/CD18), CD40, CD27, active fragments thereof, functional derivatives thereof, and combinations thereof.

A chimeric antigen receptor of the present disclosure also comprises a signaling domain that provides an intracellular signal to the immune effector cell upon antigen binding to the antigen recognition domain. The signaling domain of a chimeric antigen receptor of the present disclosure is responsible for activation of at least one of the effector functions of the immune effector cell in which the chimeric receptor is expressed. The term “effector function” refers to a specialized function of a differentiated cell. An effector function of an immune cell, for example, may be cytolytic activity or helper activity including the secretion of cytokines. An effector function in a naive, memory, or memory-type immune cell may also include antigen-dependent proliferation. Thus, the term “intracellular domain” refers to the intracellular portion of a CAR that transduces the effector function signal upon binding of an antigen to the extracellular domain and directs the immune cell to perform a specialized function. Non-limiting examples of suitable signaling domains include the zeta chain of the immune-cell receptor or any of its homologs (e.g., eta, delta, gamma, or epsilon), MB-1 chain, 829, FcRIII, FcRI, and combinations of signaling molecules, such as CD3 and CD28, CD27, 4-1BB (CD137), DNAX-activating protein 10 (DAP10), OX-40 (CD134), and combinations thereof, as well as other similar molecules and fragments. Signaling domains of other activating proteins may be used, such as FcγRIII and FcεRI. While usually the entire signaling domain will be employed, in many cases it will not be necessary to use the entire intracellular polypeptide. To that extent, a truncated portion of the signaling domain may be used as long as it still transduces the effector function signal. The term intracellular domain is also meant to include any truncated portion of the intracellular domain sufficient to transduce the effector function signal.

In further aspects, CAR-immune effector cells encompassed by the present disclosure may further comprise one or more suicide genes or a safety switch. As used herein, “suicide gene” refers to a nucleic acid sequence introduced to a CAR-immune effector cell by standard methods known in the art that, when activated, results in the death of the CAR-immune effector cell. Suicide genes may facilitate effective tracking and elimination of the CAR-immune effector cells in vivo if required. Facilitated killing by activating the suicide gene may occur by methods known in the art. Suitable suicide gene therapy systems known in the art include, but are not limited to, various the herpes simplex virus thymidine kinase (HSVtk)/ganciclovir (GCV) suicide gene therapy systems, inducible caspase 9 protein, or CD34/thymidine kinase chimeric suicide gene. In an exemplary aspect, a suicide gene is a RQR8. In another aspects, the suicide gene comprises a modified RQR8 comprising at least one CD20 mimotope, at least one CD34 epitope, or any combination thereof.

In some aspects, the suicide gene of the disclosed CAR comprises the nucleic acid sequence:

(SEQ ID NO: 11)
GCCTGCCCCTACAGCAACCCCAGCCTGTGCAGCGGCGGCGGCGGCAGCGA
GCTGCCCACCCAGGGCACCTTCAGCAACGTGAGCACCAACGTGAGCCCCG
CCAAGCCCACCACCACCGCCTGTCCTTATTCCAATCCTTCCCTGTGTAGC
GGCGGCGGCGGCAGC.

In some aspects, the suicide gene of a chimeric antigen receptor of the disclosure comprises nucleic acid sequence of SEQ ID NO: 11, wherein the said nucleic acid sequence comprises at least 60% sequence identity or similarity with nucleic acid sequence SEQ ID NO: 11. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the suicide gene of the disclosed CAR comprises the amino acid sequence:

(SEQ ID NO: 12)
ACPYSNPSLCSGGGGSELPTQGTFSNVSTNVSPAKPTTTACPYSNPSLCS
GGGGS.

In some aspects, the suicide gene of a chimeric antigen receptor of the disclosure comprises amino acid sequence of SEQ ID NO: 12, wherein the said amino acid sequence comprises at least 60% sequence identity or similarity with amino acid sequence SEQ ID NO: 12. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the CAR encompassed by the disclosure can further comprise a linker. In some aspects, the disclosed linker is a P2A. In some aspects, the disclosed P2A comprises the nucleic acid sequence:

(SEQ ID NO: 13)
GCCACCAACTTCAGCCTGCTGAAGCAGGCCGGCGACGTGGAGGAGAACCC
CGGCCCC.

In some aspects, the linker of the disclosure comprises nucleic acid sequence of SEQ ID NO: 13, wherein the said nucleic acid sequence comprises at least 60% sequence identity or similarity with nucleic acid sequence SEQ ID NO: 13. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the disclosed P2A comprises the amino acid sequence: ATNFSLLKQAGDVEENPGP (SEQ ID NO: 14).

In some aspects, the linker of the disclosure comprises amino acid sequence of SEQ ID NO: 14, wherein the said amino acid sequence comprises at least 60% sequence identity or similarity with amino acid sequence SEQ ID NO: 14. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

The disclosure further encompasses immune effector cells that express a chimeric antigen receptor. Methods for CAR design, delivery and expression in immune cells, and the manufacturing of clinical-grade CAR-immune effector cell populations are known in the art. For example, the engineered CARs may be introduced into immune effector cells using retroviruses, which efficiently and stably integrate a nucleic acid sequence encoding the chimeric antigen receptor into the target cell genome. An exemplary method for the viral vector production is described in the Methods to the Examples. Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.).

In some aspects, an immune effector cell is selected from the group consisting of a T cell, a Natural Killer (NK) cell, a cytotoxic T lymphocyte (CTL), and a regulatory T cell.

In some aspects, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. In some aspects, the immune effector cells comprise T lymphocytes.

As disclosed herein, T cells can be T cell of any subset, non-limiting examples can include T helper cells (TH cells), Cytotoxic T cells (TC cells, or CTLs), Memory T cells, Regulatory T cells (Treg cells), or Natural killer T (NKT) cells.

T helper cells (TH cells) assist other white blood cells in immunologic processes, including maturation of B cells into plasma cells and memory B cells, and activation of cytotoxic T cells and macrophages. These cells are also known as CD4⁺ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, TH9, or TFH, which secrete different cytokines to facilitate a different type of immune response.

Cytotoxic T cells (TC cells, or CTLs) destroy virally infected cells and tumor cells and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ Treg cells have been described-naturally occurring Treg cells and adaptive Treg cells.

Natural killer T (NKT) cells bridge the adaptive immune system with the innate immune system. Unlike conventional T cells that recognize peptide antigens presented by major histocompatibility complex (MHC) molecules, NKT cells recognize glycolipid antigen presented by an MHC like I, CD1d. In some aspects, NKT cells are invariant natural killer T (INKT) cells. INKT cells recognize CD1d and are restricted by their T-cell Receptor (TCR) which in humans is Vα24Jα18 and typically paired with Vβ11. INKT cells further reduce graft-versus-host disease (GVHD).

In some aspects, the T cells comprise a mixture of CD4+ cells. In other aspects, the T cells are enriched for one or more subsets based on cell surface expression. For example, in some cases, the T comprise are cytotoxic CD8+ T lymphocytes. In some embodiments, the T cells comprise γδ T cells, which possess a distinct T-cell receptor (TCR) having one γ chain and one δ chain instead of α and β chains.

Natural-killer (NK) cells are CD56+CD3− large granular lymphocytes that can kill virally infected and transformed cells and constitute a critical cellular subset of the innate immune system. Unlike cytotoxic CD8+ T lymphocytes, NK cells launch cytotoxicity against tumor cells without the requirement for prior sensitization and can also eradicate MHC-I-negative cells.

In some aspects, immune effector cells can be obtained from the subject to be treated (i.e., are autologous) and can be administered after genetic modification to express CAR with the disclosed anti-CD24 antigen recognition domain, as adoptive transfer. In such aspects, adoptive transfer comprises procuring subject's immune effector cells, followed by genetically modifying the cells to express the disclosed CARs with anti-CD24 antigen recognition domain, and infusing the modified cells back into the subject. In some aspects, immune effector cell lines or donor effector cells (allogeneic) are used. The immune effector cells for allogeneic therapy may be collected from a single subject or multiple subjects. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some aspects, immune effector cells can be isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some aspects, the expression of nucleic acids encoding CARs can be achieved by operably linking a nucleic acid encoding the CAR polypeptide comprising the disclosed anti-CD24 antigen recognition domains, to a promoter, and incorporating the construct into an expression vector (for e.g., construct disclosed FIG. 15). Typical cloning vectors contain transcription and translation terminators, initiation sequences, and promoters useful for regulation of the expression of the desired nucleic acid sequence. In some aspects, immune effector cells further include a modification of the endogenous T-cell Receptor Alpha Chain (TRAC) such that endogenous T cell receptor mediated signaling is blocked in the CAR-T cell.

In some aspects, promoters used herein comprise a strong constitutive promoter sequence. Non-limiting examples of constitutive promoter sequences include cytomegalovirus (CMV), Elongation Growth Factor-1α (EF-1α), simian virus 40 (SV40) early promoter, MND (a synthetic promoter that contains the U3 region of a modified MoMuLV LTR with myeloproliferative sarcoma virus enhancer), mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, spleen focus-forming virus (SFFV), as well as human gene promoters such as, but not limited to, the actin promoter, the myosin promoter, the hemoglobin promoter, and the creatine kinase promoter. In some aspects, the promoter can be an inducible promoter. Examples of inducible promoters include, but are not limited to a metallothionine promoter, a glucocorticoid promoter, a progesterone promoter, and a tetracycline promoter.

In some aspects, the promoter used herein is a SFFV promoter. In some aspects the SFFV promoter comprises the nucleic acid sequence:

(SEQ ID NO: 15)
GTAACGCCATTTTGCAAGGCATGGAAAAATACCAAACCAAGAATAGAGAA
GTTCAGATCAAGGGCGGGTACATGAAAATAGCTAACGTTGGGCCAAACAG
GATATCTGCGGTGAGCAGTTTCGGCCCCGGCCCGGGGCCAAGAACAGATG
GTCACCGCAGTTTCGGCCCCGGCCCGAGGCCAAGAACAGATGGTCCCCAG
ATATGGCCCAACCCTCAGCAGTTTCTTAAGACCCATCAGATGTTTCCAGG
CTCCCCCAAGGACCTGAAATGACCCTGCGCCTTATTTGAATTAACCAATC
AGCCTGCTTCTCGCTTCTGTTCGCGCGCTTCTGCTTCCCGAGCTCTATAA
AAGAGCTCACAACCCCTCACTCGGCGCGCCAGTCCTCCGACAGACTGAGT
CGCCCGGG.

In some aspects, the promoter used in the disclosure comprises nucleic acid sequence of SEQ ID NO: 15, wherein the said nucleic acid sequence comprises at least 60% sequence identity or similarity with nucleic acid sequence SEQ ID NO: 15. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the CAR comprising the disclosed anti-CD24 antigen recognition domains, can be cloned into a number of types of vectors. For example, the CAR comprising the disclosed anti-CD24 antigen recognition domains can be cloned into a vector including, but not limited to a plasmid, a phagemid, a phage derivative, an animal virus, and a cosmid. In some aspects, non-limiting examples of vectors include expression vectors, replication vectors, probe generation vectors, and sequencing vectors.

Further, the expression vector may be provided to a cell in the form of a viral vector. Viral vector technology is well known in the art. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some aspects, the polynucleotide vectors are lentiviral or retroviral vectors.

A number of viral based systems have been developed for gene transfer into mammalian cells. For example, the CAR comprising the disclosed anti-CD24 antigen recognition domain can be inserted into a vector and packaged in retroviral particles using techniques known in the art. The recombinant virus can then be isolated and delivered to cells of the subject either in vivo or ex vivo.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Non-limiting examples of selectable markers include antibiotic-resistance genes. Examples of reporter genes may include, but not limited to genes encoding luciferase, beta-galactosidase, chloramphenicol acetyl transferase, secreted alkaline phosphatase, or the green fluorescent protein gene.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art.

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some aspects, a non-viral delivery system is utilized, wherein the delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, dicetyl phosphate (“DCP”) can be obtained from K & K Laboratories; cholesterol (“Choi”) can be obtained from Calbiochem-Behring; dimyristyl phosphatidylglycerol (“DMPG”) and other lipids may be obtained from Avanti Polar Lipids, Inc.

In some aspects, the chimeric antigen receptor of the disclosure comprises anti-CD24 antigen recognition domain comprising at least two scFv, an extracellular domain, a transmembrane domain, one or more co-stimulatory domains, and an intracellular signaling domain, wherein each scFV comprises a distinct anti-CD24 antigen recognition domain. In such aspects, at least one of the scFV comprises a nucleic acid least about 80% (e.g., about 85%, about 90%, about 95%, about 98%) identity to the sequence of SEQ ID NO 7, or amino acid with at least about 80% (e.g., about 85%, about 90%, about 95%, about 98%) identity to the sequence of SEQ ID NO: 8.

In some aspects, the chimeric antigen receptor is a tandem CAR comprising at least two antigen recognition domains. In some aspects, antigen recognition domains target at least two different antigens and may be arranged in tandem. In some aspects, the at least two antigen recognition domains may be separated by linker sequences.

In some aspects, the chimeric antigen receptor of the disclosure is a tandem CAR comprising at least two antigen recognition domains, wherein the first antigen recognition domain comprises an anti-CD24 antigen recognition domain. In such aspects, the first antigen recognition domain comprises a scFV comprising a nucleic acid least about 80% (e.g., about 85%, about 90%, about 95%, about 98%) identity to the sequence of SEQ ID NO 7, or amino acid with at least about 80% (e.g., about 85%, about 90%, about 95%, about 98%) identity to the sequence of SEQ ID NO: 8.

In some aspects, the second antigen recognition domain of the disclosed tandem CAR comprises antibody or antigen-binding antibody fragment that binds to one or more of BCMA, CS1, ROR1, BAFF, CD19, CD38, ICAM-1 (CD54), CD40, CD45, CD20, syndecan 1, transferrin receptor protein 1 (TfR1), or DKK1.

In some aspects, the antigen recognition domain of the disclosed tandem CAR comprises an anti-BCMA antigen recognition domain. In some aspects, the tandem CAR comprises an anti-BCMA antigen recognition domain comprising a scFv with nucleic acid sequence:

(SEQ ID NO: 16)
GACATCGTGCTGACCCAGAGCCCCCCCAGCCTGGCCATGAGCCTGGGCAA
GAGGGCCACCATCAGCTGCAGGGCCAGCGAGAGCGTGACCATCCTGGGCA
GCCACCTGATCCACTGGTACCAGCAGAAGCCCGGCCAGCCCCCCACCCTG
CTGATCCAGCTGGCCAGCAACGTGCAGACCGGCGTGCCCGCCAGGTTCAG
CGGCAGCGGCAGCAGGACCGACTTCACCCTGACCATCGACCCCGTGGAGG
AGGACGACGTGGCCGTGTACTACTGCCTGCAGAGCAGGACCATCCCCAGG
ACCTTCGGCGGCGGCACCAAGCTGGAGATCAAGGGAGGGGGGGGATCCGG
GGGAGGAGGCTCCGGCGGAGGCGGAAGCCAGATCCAGCTGGTGCAGAGCG
GCCCCGAGCTGAAGAAGCCCGGCGAGACCGTGAAGATCAGCTGCAAGGCC
AGCGGCTACACCTTCACCGACTACAGCATCAACTGGGTGAAGAGGGCCCC
CGGCAAGGGCCTGAAGTGGATGGGCTGGATCAACACCGAGACCAGGGAGC
CCGCCTACGCCTACGACTTCAGGGGCAGGTTCGCCTTCAGCCTGGAGACC
AGCGCCAGCACCGCCTACCTGCAGATCAACAACCTGAAGTACGAGGACAC
CGCCACCTACTTCTGCGCCCTGGACTACAGCTACGCCATGGACTACTGGG
GCCAGGGCACCAGCGTGACCGTGAGCAGC.

In some aspects, an anti-BCMA antigen recognition domain of a tandem CAR of the disclosure comprises a scFv, comprising nucleic acid sequence of SEQ ID NO: 16, wherein the said nucleic acid sequence comprises at least 60% sequence identity or similarity with nucleic acid sequence SEQ ID NO: 16. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the tandem CAR comprises an anti-BCMA antigen recognition domain comprising a scFv with amino acid sequence:

(SEQ ID NO: 17)
DIVLTQSPPSLAMSLGKRATISCRASESVTILGSHLIHWYQQKPGQPPTL
LIQLASNVQTGVPARFSGSGSRTDFTLTIDPVEEDDVAVYYCLQSRTIPR
TFGGGTKLEIKGGGGSGGGGSGGGGSQIQLVQSGPELKKPGETVKISCKA
SGYTFTDYSINWVKRAPGKGLKWMGWINTETREPAYAYDFRGRFAFSLET
SASTAYLQINNLKYEDTATYFCALDYSYAMDYWGQGTSVTVSS.

In some aspects, an anti-BCMA antigen recognition domain of a tandem CAR of the disclosure comprises a scFv, comprising amino acid sequence of SEQ ID NO: 17, wherein the said nucleic acid sequence comprises at least 60% sequence identity or similarity with nucleic acid sequence SEQ ID NO: 17. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the tandem CAR comprises a first antigen recognition domain comprising an anti-CD24 antigen recognition domain, and a second antigen recognition domain comprising an anti-BCMA antigen recognition domain. In such aspects, the tandem CAR comprises an anti-CD24 antigen recognition domain comprising a scFv comprising a nucleic acid at least about 80% (e.g., about 85%, about 90%, about 95%, about 98%) identity to the sequence of SEQ ID NO: 7, or scFv with amino acid at least about 80% (e.g., about 85%, about 90%, about 95%, about 98%) identity to the sequence of SEQ ID NO: 8, and an anti-BCMA antigen recognition domain comprising a scFv with nucleic acid sequence at least about 80% (e.g., about 85%, about 90%, about 95%, about 98%) identity to the sequence of SEQ ID NO: 16, or scFv with amino acid at least about 80% (e.g., about 85%, about 90%, about 95%, about 98%) identity to the sequence of SEQ ID NO: 17.

In some aspects, the tandem CAR comprises an anti-CD24 antigen recognition domain comprising a scFv comprising a nucleic acid with 100% identity to the sequence of SEQ ID NO: 7, or scFv with amino acid with 100% identity to the sequence of SEQ ID NO: 8, and an anti-BCMA antigen recognition domain comprising a scFv with nucleic acid sequence with 100% identity to the sequence of SEQ ID NO: 16, or scFv with amino acid with 100% identity to the sequence of SEQ ID NO: 17.

In some aspects, the chimeric antigen receptor of the disclosure is a bispecific chimeric antigen receptor. In such aspects, the chimeric antigen receptor comprises a first antigen recognition domain comprising an anti-CD24 antigen recognition domain disclosed herein and a second antigen recognition domain comprising antibodies or antigen-binding antibody fragments that bind to an antigen expressed by malignant B cell and/or plasma cell. In such aspects, the second antigen recognition domain comprises antibody or antigen-binding antibody fragment that binds to one or more of BCMA, CS1, ROR1, BAFF, CD19, CD38, ICAM-1 (CD54), CD40, CD45, CD20, syndecan 1, transferrin receptor protein 1 (TfR1), or DKK1. In some aspects, the at least two antigen recognition domains may be separated by linker sequences. In some aspects, the linker is poly-Glycine-Serine (G4S) 4 peptide linker.

In some aspects, the bispecific chimeric antigen receptor of the disclosure comprises a first antigen recognition domain comprising an anti-CD24 antigen recognition domain. In some aspects, the anti-CD24 antigen recognition domain comprises an scFv comprising a nucleic acid at least about 80% (e.g., about 85%, about 90%, about 95%, about 98%) identity to the sequence of SEQ ID NO: 7, or scFv with amino acid at least about 80% (e.g., about 85%, about 90%, about 95%, about 98%) identity to the sequence of SEQ ID NO: 8.

In some aspects, the bispecific chimeric antigen receptor of the disclosure comprises an antigen recognition domain comprising an anti-BCMA antigen recognition domain. In some aspects, an anti-BCMA antigen recognition domain comprises an scFv with nucleic acid sequence at least about 80% (e.g., about 85%, about 90%, about 95%, about 98%) identity to the sequence of SEQ ID NO: 16, or scFv with amino acid at least about 80% (e.g., about 85%, about 90%, about 95%, about 98%) identity to the sequence of SEQ ID NO: 17.

In some aspects, the bispecific chimeric antigen receptor comprises a first antigen recognition domain comprising an anti-CD24 antigen recognition domain, and a second antigen recognition domain comprising an anti-BCMA antigen recognition domain. In such aspects, the bispecific CAR comprises an anti-CD24 antigen recognition domain comprising an scFv comprising a nucleic acid at least about 80% (e.g., about 85%, about 90%, about 95%, about 98%) identity to the sequence of SEQ ID NO: 7, or scFv with amino acid at least about 80% (e.g., about 85%, about 90%, about 95%, about 98%) identity to the sequence of SEQ ID NO: 8, and an anti-BCMA antigen recognition domain comprising an scFv with nucleic acid sequence at least about 80% (e.g., about 85%, about 90%, about 95%, about 98%) identity to the sequence of SEQ ID NO: 16, or scFv with amino acid at least about 80% (e.g., about 85%, about 90%, about 95%, about 98%) identity to the sequence of SEQ ID NO: 17.

In some aspects, the bispecific chimeric antigen receptor comprises an anti-CD24 antigen recognition domain comprising an scFv comprising a nucleic acid with 100% identity to the sequence of SEQ ID NO: 7, or scFv with amino acid with 100% identity to the sequence of SEQ ID NO: 8, and an anti-BCMA antigen recognition domain comprising an scFv with nucleic acid sequence with 100% identity to the sequence of SEQ ID NO: 16, or scFv with amino acid with 100% identity to the sequence of SEQ ID NO: 17.

### III. Antibodies

In some aspects, the present disclosure further encompasses a bispecific antibody, a multi-specific antibody, or a hybrid antibody having at least two distinct binding domains with different specificities, with at least one domain comprising an anti-CD24 antigen recognition domain disclosed herein. In some aspects, a bispecific antibody, a multi-specific antibody, or a hybrid antibody comprises a anti-CD24 scFv disclosed herein, and at least one of an immune cell engager (e.g., a T cell engager, an NK cell engager, an iNKT cell engager, a B cell engager, a dendritic cell engager, or a macrophage cell engager). Such antibody constructs are recombinant protein constructs made from two flexibly linked antibody derived binding domains, with one binding domain specific for a selected tumor antigen on target tumor cells and the second binding domain that binds to and/or activates T cells, NK cells, INKT cells, B cells, dendritic cells, or macrophage cells. In some aspects, the disclosed antibodies can transiently direct immune cells to the target tumor cells and/or activate the inherent cytolytic potential of immune cells against target tumor cells. In some aspects, a bispecific antibody, a multi-specific antibody, or a hybrid antibody comprises with at least one domain comprising an anti-CD24 scFv disclosed herein, and a second domain comprising a binding domain specific for an antigen expressed by B cell and/or plasma cell tumor cells. A bispecific antibody, a multi-specific antibody, or a hybrid antibody construct can be produced by a variety of methods, including fusion of hybridomas, linking of Fab′ fragments, or any other known method in the art.

In some aspects, the bispecific antibody, the multi-specific antibody, or the hybrid antibody comprises a T-cell engager (e.g., bispecific T cell engager (BITE)) comprising first antigen recognition domain comprising anti-CD24 scFv disclosed herein, and a second antigen recognition domain comprising an antibody or antigen-binding antibody fragment that bind to or activate T cells. In such aspects, a second antigen recognition domain comprising antibody or antigen-binding antibody fragment binds to one or more of CD2, CD3, CD4, CD5, CD6, CD8, CD25, CD27, CD28, CD30, CD40, CD40L, CD44, CD45, CD69, CD90, CRa, TCRp, TCRy, TCRC, ICOS, HVEM, LIGHT, 4-IBB, OX40, DR3, GITR, TIMI, SLAM, or CD226

In some aspects, the bispecific antibody, the multi-specific antibody, or the hybrid antibody has a binding domain that engages NK cells. In such aspects, the bispecific antibody comprises a first antigen recognition domain comprising anti-CD24 scFv disclosed herein, and a second antigen recognition domain comprising an antibody or antigen-binding antibody fragments that bind to or activate NK cells. In such aspects, a second antigen recognition domain comprising an antibody or antigen-binding antibody fragment binds to one or more of CD16 (e.g., CD16a, CD16b, or both), NKp30, NKp40, NKp44, NKp46, NKG2D, DNAMI, DAP10, CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS 1, KIR2DS3, KIR2DS5, KIR2DS1, CD94, NKG2C, NKG2E, or CD160.

In some aspects, the bispecific antibody, the multi-specific antibody, or the hybrid antibody has a binding domain that engages INKT cells. In such aspects, the bispecific antibody comprises a first antigen recognition domain comprising anti-CD24 scFv disclosed herein, and a second antigen recognition domain comprising an antibody or antigen-binding antibody fragments that bind to or activate iNKT cells. In such aspects, a second antigen recognition domain comprising an antibody or antigen-binding antibody fragment binds to CD3.

In some aspects, the bispecific antibody, the multi-specific antibody, or the hybrid antibody has a binding domain that engages B cells. In such aspects, the bispecific antibody comprises a first antigen recognition domain comprising anti-CD24 scFv disclosed herein, and a second antigen recognition domain comprising an antibody or antigen-binding antibody fragments that bind to or activate B cells. In such aspects, a second antigen recognition domain comprising an antibody or antigen-binding antibody fragment binds to one or more of OX40, CD40 or CD70.

In some aspects, the bispecific antibody, the multi-specific antibody, or the hybrid antibody has a binding domain that engages dendritic cells. In such aspects, the bispecific antibody comprises a first antigen recognition domain comprising anti-CD24 scFv disclosed herein, and a second antigen recognition domain comprising an antibody or antigen-binding antibody fragments that bind to or activate dendritic cells. In such aspects, a second antigen recognition domain comprising an antibody or antigen-binding antibody fragment binds to one or more of CD2, OX40, 4-IBB, TLR4, CD47, or STING.

In some aspects, the bispecific antibody, the multi-specific antibody, or the hybrid antibody has a binding domain that engages macrophage cell. In such aspects, the bispecific antibody comprises a first antigen recognition domain comprising anti-CD24 scFv disclosed herein, and a second antigen recognition domain comprising an antibody or antigen-binding antibody fragments that bind to or activate macrophage cells. In such aspects, a second antigen recognition domain comprising an antibody or antigen-binding antibody fragment binds to one or more of OX40, CD40, CD70, TLR4, TLR9, CD47 or STING agonist.

In some aspects, a bispecific antibody, a multi-specific antibody, or a hybrid antibody comprises a first antigen recognition domain comprising anti-CD24 scFv disclosed herein, and a second antigen recognition domain comprising an antibody or antigen-binding antibody fragments that bind to an antigen expressed by B cell and/or plasma cell tumor cells. In such aspects, a second antigen recognition domain comprising an antibody or antigen-binding antibody fragment binds to one or more of BCMA, CS1, ROR1, BAFF, CD19, CD38, ICAM-1 (CD54), CD40, CD45, CD20, syndecan 1, transferrin receptor protein 1 (TfR1), or DKK1. In some aspects, the second antigen recognition domain comprises an antibody or antigen-binding antibody fragment which binds to BCMA. In some aspects, the second antigen recognition domain comprises a scFv with nucleic acid sequence at least about 80% (e.g., about 85%, about 90%, about 95%, about 98%) identity to the sequence of SEQ ID NO: 16, or scFv with amino acid at least about 80% (e.g., about 85%, about 90%, about 95%, about 98%) identity to the sequence of SEQ ID NO: 17.

In some aspects, the disclosed bispecific antibody, a multi-specific antibody, or a hybrid antibody comprises an anti-CD24 antigen recognition domain comprising a scFv, comprising a nucleic acid at least about 80% (e.g., about 85%, about 90%, about 95%, about 98%) identity to the sequence of SEQ ID NO: 7, or scFv with amino acid at least about 80% (e.g., about 85%, about 90%, about 95%, about 98%) identity to the sequence of SEQ ID NO: 8. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the disclosed bispecific antibody, a multi-specific antibody, or a hybrid antibody comprises an anti-CD24 antigen recognition domain comprising a scFv a nucleic acid with 100% identity to the sequence of SEQ ID NO: 7, or scFv with amino acid with 100% identity to the sequence of SEQ ID NO: 8.

In some aspects, the second antigen recognition domain of the disclosed bispecific antibody, a multi-specific antibody, or a hybrid antibody comprises an antibody or antigen-binding antibody fragment which binds to BCMA. In some aspects, the second antigen recognition domain comprises a scFv with nucleic acid sequence at least about 80% (e.g., about 85%, about 90%, about 95%, about 98%) identity to the sequence of SEQ ID NO: 16, or scFv with amino acid at least about 80% (e.g., about 85%, about 90%, about 95%, about 98%) identity to the sequence of SEQ ID NO: 17.

In some aspects, the disclosed bispecific antibody, a multi-specific antibody, or a hybrid antibody comprises an anti-BCM antigen recognition domain comprising a scFv a nucleic acid with 100% identity to the sequence of SEQ ID NO: 16, or scFv with amino acid with 100% identity to the sequence of SEQ ID NO: 17.

In some aspects, the disclosed bispecific antibody, a multi-specific antibody, or a hybrid antibody comprises a first antigen recognition domain comprising an anti-CD24 antigen recognition domain, and a second antigen recognition domain comprising an anti-BCMA antigen recognition domain. In such aspects, the disclosed bispecific antibody, a multi-specific antibody, or a hybrid antibody comprises an anti-CD24 antigen recognition domain comprising an scFv comprising a nucleic acid at least about 80% (e.g., about 85%, about 90%, about 95%, about 98%) identity to the sequence of SEQ ID NO: 7, or scFv with amino acid at least about 80% (e.g., about 85%, about 90%, about 95%, about 98%) identity to the sequence of SEQ ID NO: 8, and an anti-BCMA antigen recognition domain comprising an scFv with nucleic acid sequence at least about 80% (e.g., about 85%, about 90%, about 95%, about 98%) identity to the sequence of SEQ ID NO: 16, or scFv with amino acid at least about 80% (e.g., about 85%, about 90%, about 95%, about 98%) identity to the sequence of SEQ ID NO: 17.

In some aspects, the disclosed bispecific antibody, a multi-specific antibody, or a hybrid antibody comprises an anti-CD24 antigen recognition domain comprising an scFv comprising a nucleic acid with 100% identity to the sequence of SEQ ID NO: 7, or scFv with amino acid with 100% identity to the sequence of SEQ ID NO: 8, and an anti-BCMA antigen recognition domain comprising an scFv with nucleic acid sequence with 100% identity to the sequence of SEQ ID NO: 16, or scFv with amino acid with 100% identity to the sequence of SEQ ID NO: 17.

### IV. Methods of Using CAR-expressing Cells and Antibodies

The current disclosure further encompasses treatment of cancer. In some aspects, the present disclosure provides a method of killing a malignant cell, the method comprising contacting the malignant cell with an effective amount of an immune effector cell comprising a chimeric antigen receptor (CAR-immune effector cell), wherein the CAR-immune effector cell is deficient in an antigen to which the chimeric antigen receptor specifically binds, and wherein the chimeric antigen receptor specifically binds an antigen expressed on a malignant cell. In various aspects, the malignant cell is a Burkitt lymphoma, chronic lymphocytic leukemia/small lymphocytic lymphoma (CLL/SLL), diffuse large B-cell lymphoma, follicular lymphoma, or mantle cell lymphoma, Monoclonal gammopathy of undetermined significance, Lymphoplasmacytic lymphoma, Plasmacytoma, or myeloma. In further aspects, the antigen is CD24. Suitable CAR-immune effector cells are described in detail in Section II.

Contacting a malignant cell with an effective amount of a CAR-immune effector cell generally involves admixing the CAR-immune effector cell and the malignant cell for a period of time sufficient to allow the chimeric antigen receptor of the CAR-immune effector cell to bind its cognate antigen on the surface of the malignant cell. This may occur in vitro or ex vivo. The term “effective amount”, as used herein, means an amount that leads to measurable effect, e.g., antigen-dependent cell proliferation, cytokine secretion, cytotoxic killing, etc. The effective amount may be determined by using the methods known in the art and/or described in further detail in the examples.

In another aspect, the present disclosure provides a method for treating a subject having a B cell malignancy. In some aspects, the B cell malignancy is a Burkitt lymphoma, chronic lymphocytic leukemia/small lymphocytic lymphoma (CLL/SLL), diffuse large B-cell lymphoma, follicular lymphoma, or mantle cell lymphoma. The method comprises administering to the subject a therapeutically effective amount of plurality of CAR-immune effector cells, each CAR-immune effector cell comprising the same chimeric antigen receptor, optionally wherein the CAR-immune effector cells are deficient in an antigen specifically recognized by the chimeric antigen receptor, and wherein the chimeric antigen receptor specifically binds an antigen expressed on a malignant B cell. In some aspects, the antigen expressed on the malignant B cell is CD24. In some aspects, the method for treating a subject having a B cell malignancy comprises administering to the subject a therapeutically effective amount of plurality of CAR-immune effector cells, each CAR-immune effector cell comprising the same chimeric antigen receptor, and wherein the chimeric antigen receptor specifically binds to CD24. In some aspects, the method for treating a subject having a B cell malignancy, comprises administering to the subject a therapeutically effective amount of plurality of CAR-immune effector cells, each CAR-immune effector cell comprising the same chimeric antigen receptor, and wherein the chimeric antigen receptor comprises an anti-CD24 antigen recognition domain disclosed herein.

In further aspects, the method for treating a subject having a B cell malignancy, disclosed herein comprises administering to the subject a therapeutically effective amount of plurality of CAR-immune effector cells, wherein the chimeric antigen receptor is a tandem CAR with first antigen recognition domain comprising an anti-CD24 antigen recognition domain, and a second antigen recognition domain comprising an anti-BCMA antigen recognition domain. In some aspects, the method for treating a subject having a B cell malignancy, comprises administering to the subject a therapeutically effective amount of plurality of CAR-immune effector cells, each CAR-immune effector cell comprising the same tandem chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In some aspects, the method for treating a subject having a B cell malignancy, disclosed herein comprises administering to the subject a therapeutically effective amount of plurality of CAR-immune effector cells, wherein the chimeric antigen receptor is a bispecific CAR with first antigen recognition domain comprising an anti-CD24 antigen recognition domain, and a second antigen recognition domain comprising an anti-BCMA antigen recognition domain. In some aspects, the method for treating a subject having a B cell malignancy, comprises administering to the subject a therapeutically effective amount of plurality of CAR-immune effector cells, each CAR-immune effector cell comprising the same bispecific chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In some aspects, the present disclosure provides a method for treating a subject having a plasma cell malignancy. In some aspects, the plasma cell malignancy is Monoclonal gammopathy of undetermined significance (MGUS), Lymphoplasmacytic lymphoma, Plasmacytoma, or multiple myeloma. The method comprises administering to the subject a therapeutically effective amount of plurality of CAR-immune effector cells, each CAR-immune effector cell comprising the same chimeric antigen receptor, optionally wherein the CAR-immune effector cells are deficient in an antigen specifically recognized by the chimeric antigen receptor, and wherein the chimeric antigen receptor specifically binds an antigen expressed on a malignant plasma cell. In some aspects, the antigen expressed on the malignant plasma cell is CD24. In some aspects, the method for treating a subject having a plasma cell malignancy comprises administering to the subject a therapeutically effective amount of plurality of CAR-immune effector cells, each CAR-immune effector cell comprising the same chimeric antigen receptor, and wherein the chimeric antigen receptor specifically binds to CD24. In some aspects, the method for treating a subject having a plasma cell malignancy comprises, administering to the subject a therapeutically effective amount of plurality of CAR-immune effector cells, each CAR-immune effector cell comprising the same chimeric antigen receptor, and wherein the chimeric antigen receptor comprises an anti-CD24 antigen recognition domain disclosed herein.

In further aspects, the method for treating a subject having plasma cell malignancy, disclosed herein comprises administering to the subject a therapeutically effective amount of plurality of CAR-immune effector cells, wherein the chimeric antigen receptor is a tandem CAR with first antigen recognition domain comprising an anti-CD24 antigen recognition domain, and a second antigen recognition domain comprising an anti-BCMA antigen recognition domain. In some aspects, the method for treating a subject having a plasma cell malignancy, comprises administering to the subject a therapeutically effective amount of plurality of CAR-immune effector cells, each CAR-immune effector cell comprising the same tandem chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In some aspects, the method for treating a subject having a plasma cell malignancy, disclosed herein comprises administering to the subject a therapeutically effective amount of plurality of CAR-immune effector cells, wherein the chimeric antigen receptor is a bispecific CAR with first antigen recognition domain comprising an anti-CD24 antigen recognition domain, and a second antigen recognition domain comprising an anti-BCMA antigen recognition domain. In some aspects, the method for treating a subject having a plasma cell malignancy, comprises administering to the subject a therapeutically effective amount of plurality of CAR-immune effector cells, each CAR-immune effector cell comprising the same bispecific chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In certain aspects, the present disclosure provides a method for treating a subject having multiple myeloma. The method comprises administering to the subject a therapeutically effective amount of plurality of CAR-immune effector cells, each CAR-immune effector cell comprising the same chimeric antigen receptor, optionally wherein the CAR-immune effector cells are deficient in an antigen specifically recognized by the chimeric antigen receptor, and wherein the chimeric antigen receptor specifically binds an antigen expressed on multiple myeloma cells. In some aspects, the antigen expressed on the multiple myeloma cell is CD24. In some aspects, the method of treating a subject having multiple myeloma comprises administering to the subject a therapeutically effective amount of plurality of CAR-immune effector cells, each CAR-immune effector cell comprising the same chimeric antigen receptor, and wherein the chimeric antigen receptor specifically binds to CD24. In some aspects, the method of treating a subject having multiple myeloma comprises, administering to the subject a therapeutically effective amount of plurality of CAR-immune effector cells, each CAR-immune effector cell comprising the same chimeric antigen receptor, and wherein the chimeric antigen receptor comprises an anti-CD24 antigen recognition domain disclosed herein.

In further aspects, the method for treating a subject having multiple myeloma, disclosed herein, comprises administering to the subject a therapeutically effective amount of plurality of CAR-immune effector cells, wherein the chimeric antigen receptor is a tandem CAR with first antigen recognition domain comprising an anti-CD24 antigen recognition domain, and a second antigen recognition domain comprising an anti-BCMA antigen recognition domain. In some aspects, the method for treating a subject having multiple myeloma, comprises administering to the subject a therapeutically effective amount of plurality of CAR-immune effector cells, each CAR-immune effector cell comprising the same tandem chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In some aspects, the method for treating a subject having multiple myeloma, disclosed herein comprises administering to the subject a therapeutically effective amount of plurality of CAR-immune effector cells, wherein the chimeric antigen receptor is a bispecific CAR with first antigen recognition domain comprising an anti-CD24 antigen recognition domain, and a second antigen recognition domain comprising an anti-BCMA antigen recognition domain. In some aspects, the method for treating a subject having multiple myeloma, comprises administering to the subject a therapeutically effective amount of plurality of CAR-immune effector cells, each CAR-immune effector cell comprising the same bispecific chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In some aspects, the present disclosure provides a method for treating a subject having a malignant cell that express CD24. The method comprises administering to the subject a therapeutically effective amount of plurality of CAR-immune effector cells, each CAR-immune effector cell comprising the same chimeric antigen receptor, optionally wherein the CAR-immune effector cells are deficient in an antigen specifically recognized by the chimeric antigen receptor, and wherein the chimeric antigen receptor specifically binds CD24. In some aspects, the method of treating a subject having a malignant cell that express CD24 comprises administering to the subject a therapeutically effective amount of plurality of CAR-immune effector cells, each CAR-immune effector cell comprising the same chimeric antigen receptor, and wherein the chimeric antigen receptor comprises an anti-CD24 antigen recognition domain disclosed herein.

In further aspects, the method for treating a subject having a malignant cell that express CD24, disclosed herein, comprises administering to the subject a therapeutically effective amount of plurality of CAR-immune effector cells, wherein the chimeric antigen receptor is a tandem CAR with first antigen recognition domain comprising an anti-CD24 antigen recognition domain, and a second antigen recognition domain comprising an anti-BCMA antigen recognition domain. In some aspects, the method for treating a subject having a malignant cell that express CD24, comprises administering to the subject a therapeutically effective amount of plurality of CAR-immune effector cells, each CAR-immune effector cell comprising the same tandem chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In some aspects, the method for treating a subject having a malignant cell that express CD24, disclosed herein comprises administering to the subject a therapeutically effective amount of plurality of CAR-immune effector cells, wherein the chimeric antigen receptor is a bispecific CAR with first antigen recognition domain comprising an anti-CD24 antigen recognition domain, and a second antigen recognition domain comprising an anti-BCMA antigen recognition domain. In some aspects, the method for treating a subject having a malignant cell that express CD24, comprises administering to the subject a therapeutically effective amount of plurality of CAR-immune effector cells, each CAR-immune effector cell comprising the same bispecific chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In some aspects, the present disclosure provides a method for treating a subject having a refractory or relapsed B cell or plasma cell malignancy. The method comprises administering to the subject a therapeutically effective amount of plurality of CAR-immune effector cells, each CAR-immune effector cell comprising the same chimeric antigen receptor, optionally wherein the CAR-immune effector cells are deficient in an antigen specifically recognized by the chimeric antigen receptor, and wherein the chimeric antigen receptor specifically binds CD24. In some aspects, the method of treating a subject having a refractory or relapsed B cell or plasma cell malignancy comprises administering to the subject a therapeutically effective amount of plurality of CAR-immune effector cells, each CAR-immune effector cell comprising the same chimeric antigen receptor, and wherein the chimeric antigen receptor comprises an anti-CD24 antigen recognition domain disclosed herein.

In further aspects, the method for treating a subject having a refractory or relapsed B cell or plasma cell malignancy, disclosed herein, comprises administering to the subject a therapeutically effective amount of plurality of CAR-immune effector cells, wherein the chimeric antigen receptor is a tandem CAR with first antigen recognition domain comprising an anti-CD24 antigen recognition domain, and a second antigen recognition domain comprising an anti-BCMA antigen recognition domain. In some aspects, the method for treating a subject having a refractory or relapsed B cell or plasma cell malignancy, comprises administering to the subject a therapeutically effective amount of plurality of CAR-immune effector cells, each CAR-immune effector cell comprising the same tandem chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In some aspects, the method for treating a subject having a refractory or relapsed B cell or plasma cell malignancy, disclosed herein comprises administering to the subject a therapeutically effective amount of plurality of CAR-immune effector cells, wherein the chimeric antigen receptor is a bispecific CAR with first antigen recognition domain comprising an anti-CD24 antigen recognition domain, and a second antigen recognition domain comprising an anti-BCMA antigen recognition domain. In some aspects, the method for treating a subject having a refractory or relapsed B cell or plasma cell malignancy, comprises administering to the subject a therapeutically effective amount of plurality of CAR-immune effector cells, each CAR-immune effector cell comprising the same bispecific chimeric antigen receptor, and wherein the chimeric antigen receptor comprises first antigen recognition domain comprising an anti-CD24 antigen recognition domain, and a second antigen recognition domain comprising an anti-BCMA antigen recognition domain, disclosed herein.

In some aspects, the refractory or relapsed B cell or plasma cell malignancy is refractory/relapsed multiple myeloma and the method comprises administering to a subject in need thereof, a therapeutically effective amount of plurality of CAR-immune effector cells, wherein the chimeric antigen receptor is a CAR with antigen recognition domain comprising an anti-CD24 antigen recognition domain.

In some aspects, the refractory or relapsed B cell or plasma cell malignancy is refractory/relapsed multiple myeloma and the method comprises administering to a subject in need thereof, a therapeutically effective amount of plurality of CAR-immune effector cells, wherein the chimeric antigen receptor is a tandem CAR with first antigen recognition domain comprising an anti-CD24 antigen recognition domain, and a second antigen recognition domain comprising an anti-BCMA antigen recognition domain.

In some aspects, the refractory or relapsed B cell or plasma cell malignancy is refractory/relapsed multiple myeloma and the method comprises administering to a subject in need thereof, a therapeutically effective amount of plurality of CAR-immune effector cells, wherein the chimeric antigen receptor is a bispecific CAR with first antigen recognition domain comprising an anti-CD24 antigen recognition domain, and a second antigen recognition domain comprising an anti-BCMA antigen recognition domain.

The disclosed CAR-immune effector cells may be administered in effective doses. The effective dose may be either one or multiple doses and are sufficient to produce the desired therapeutic effect. In some aspects, the dosage of CAR-immune effector cells may range from about 1×10⁴ to 5×10⁹ cells/Kg body weight of subject receiving therapy. In some aspects, a pharmaceutical composition comprising the immune effector cells described herein may be administered at a dosage of 1×10⁴ to 2×10⁹ cells/kg body weight, such as 1×10⁵ to 2×10⁶ cells/kg body weight. Immune effector cells compositions may also be administered multiple times at these dosages. The immune effector cells can be administered by using infusion techniques that are commonly known in immunotherapy. The optimal dosage and treatment regime for a particular subject can readily be determined by one skilled in the art of medicine by monitoring the subject for signs of disease and adjusting the treatment accordingly. Further, the effective dose may be calculated based on the stage of the malignancy, the health of the subject, and the type of malignancy. In the situation where multiple doses are administered, that dose and the interval between the doses may be determined based on the subject's response to therapy.

In some aspects, treatment with disclosed CAR-immune effector cells reduces tumor burden in a subject. In some aspects, reduction in tumor burden in a subject, comprises reduction in number of malignant cells, reduction in tumor size, and/or reduction in the tumor growth. Tumor burden in a subject can be assessed using any conventional methods known in the art, non-limiting examples of which include, diagnostic techniques such as X ray, CT scan, bone marrow biopsy, serum protein electrophoresis (SPEP), or MRI. In some aspects, administration of disclosed CAR-immune effector cells reduces number of malignant cells, tumor size, and/or tumor growth in the subject, by at least 10%, e.g., 20%, 30%, 40%, 50%, 60%, 70%, 80%, 90%, 95% or above, as compared to number of malignant cells, tumor size, and/or tumor growth before administration of the inhibitor (baseline) or a control subject not receiving the treatment.

In some aspects, treatment with disclosed CAR-immune effector cells enhances survival and prolongs life of a subject. Survival of the subject can be determined using known methods in the art, non-limiting examples of which include Kaplan Meier analysis, Gleason score, Log-rank (Mantel-Cox) test, and Cox hazard analysis. In some aspects, administration of disclosed CAR-immune effector cells in the subject enhances survival, by at least 10%, e.g., 20%, 30%, 40%, 50%, 60%, 70%, 80%, 90%, 95% or above, as compared to survival of a control subject not receiving the treatment or expected survival of the subject before the treatment.

In some aspects, treatment disclosed CAR-immune effector cells enhances the death of a malignant cell. Tumor cell death may be determined using any known methods in the art for measuring cell apoptosis or cell viability, including bioluminescent imaging or flow cytometry. In some aspects, administration of disclosed CAR-immune effector cells enhances death of malignant cells in the subject, by at least 10%, e.g., 20%, 30%, 40%, 50%, 60%, 70%, 80%, 90%, 95% or above, as compared to malignant cells, before administration of the inhibitor (baseline) or a control subject not receiving the treatment.

The administration of the disclosed immune effector cells, or compositions thereof, may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some aspects, the disclosed immune effector cells, or compositions thereof, are administered to a patient by intradermal or subcutaneous injection. In some aspects, the disclosed compositions are administered by i.v. injection. The immune effector cells, or compositions thereof, may also be injected directly into a tumor, lymph node, or site of infection. In some aspects of the method, compositions disclosed herein are formulated for intravenous administration.

The methods described herein, encompasses allogenic CAR-immune effector cell therapy or autologous CAR-immune effector cell therapy. In some aspects, the method comprise adoptive transfer of disclosed CAR-immune effector cells. Immune effector cells procured from the subject in need of the treatment, are genetically modified to express the CARs comprising disclosed anti-CD24 antigen recognition domain (for e.g., CAR with anti-CD24 antigen recognition domain, a tandem CAR with anti-CD24 and anti-BCMA antigen recognition domains, or a bispecific CAR with anti-CD24 and anti-BCMA antigen recognition domains) and are administered back into the subject. In some aspects, allogenic immune effector cells are used in the methods described herein. Allogenic sources can comprise of immune effector cell lines or donor effector cells. Once procured, allogenic immune effector cells are genetically modified to express the CARs comprising disclosed anti-CD24 antigen recognition domain (for e.g., CAR with anti-CD24 antigen recognition domain, a tandem CAR with anti-CD24 and anti-BCMA antigen recognition domains, or a bispecific CAR with anti-CD24 and anti-BCMA antigen recognition domains) and are administered into a subject in need thereof.

Further provided herein, is a method of treating a B cell malignancy, or a plasma cell malignancy, in a subject in need thereof, using one or more of the disclosed bispecific antibody, multi-specific antibody, or hybrid antibody. In some aspects, the B cell malignancy is a Burkitt lymphoma, chronic lymphocytic leukemia/small lymphocytic lymphoma (CLL/SLL), diffuse large B-cell lymphoma, follicular lymphoma, or mantle cell lymphoma and the method comprises administering to the subject a therapeutically effective amount of one or more of the disclosed bispecific antibody, multi-specific antibody, or hybrid antibody. In some aspects, the plasma cell malignancy is Monoclonal gammopathy of undetermined significance (MGUS), Lymphoplasmacytic lymphoma, Plasmacytoma, or multiple myeloma and the method comprises administering to the subject a therapeutically effective amount of one or more of the disclosed bispecific antibody, multi-specific antibody, or hybrid antibody. In some aspects, the malignancy is multiple myeloma and the method comprises administering to the subject a therapeutically effective amount of one or more of the disclosed bispecific antibody, multi-specific antibody, or hybrid antibody.

In further aspects, the method comprises treating a subject having a refractory or relapsed B cell or plasma cell malignancy. The method comprises administering to the subject a therapeutically effective amount of one or more of the disclosed bispecific antibody, multi-specific antibody, or hybrid antibody. In some aspects, the refractory or relapsed B cell or plasma cell malignancy is refractory/relapsed multiple myeloma and the method comprises administering to a subject in need thereof, a therapeutically effective amount of one or more of the bispecific antibody, multi-specific antibody, or hybrid antibody, disclosed herein.

In some aspects, the therapeutically effective amount of bispecific antibody, a multi-specific antibody, or a hybrid antibody is between about 5 mg to about 1000 mg per day with unit doses ranging from about 1 mg to about 250 mg. In some aspects, the effective amount is about 0.001-10 mg/kg, such as about 0.001-5 mg/kg, for example about 0.001-2 mg/kg, such as about 0.001-1 mg/kg, for instance about 0.001, about 0.01, about 0.1, about 1 or about 10 mg/kg. In general, the physician may determine the appropriate dosage depending on the age, weight and any other factors specific to the subject to be treated.

The disclosed compositions comprising bispecific antibody, multi-specific antibody, or hybrid antibody described herein, can be formulated for administration as various formulations including oral, parenteral (e.g. intravenous, intramuscular, intraperinoneal, subcutaneous), intravenously as a bolus or by continuous infusion over a period of time, injected by intramuscular, subcutaneous, intra-articular, intrasynovial, intratumoral, peritumoral, intralesional, or perilesional routes, to exert local as well as systemic therapeutic effects.

In some aspects, administration of therapeutically effective amount of bispecific antibody, multi-specific antibody, or hybrid antibody described herein, can reduces tumor burden in a subject. In some aspects, reduction in tumor burden in a subject, comprises reduction in number of malignant cells, reduction in tumor size, and/or reduction in the tumor growth. Tumor burden in a subject can be assessed using any conventional methods known in the art, non-limiting examples of which include, diagnostic techniques such as X ray, CT scan, bone marrow biopsy, serum protein electrophoresis (SPEP), or MRI. In some aspects, administration of disclosed bispecific antibody, a multi-specific antibody, or a hybrid antibody reduces number of malignant cells, tumor size, and/or tumor growth in the subject, by at least 10%, e.g., 20%, 30%, 40%, 50%, 60%, 70%, 80%, 90%, 95% or above, as compared to number of malignant cells, tumor size, and/or tumor growth before administration of the inhibitor (baseline) or a control subject not receiving the treatment.

In some aspects, treatment with the disclosed bispecific antibody, multi-specific antibody, or hybrid antibody enhances survival and prolongs life of a subject. Survival of the subject can be determined using known methods in the art, non-limiting examples of which include Kaplan Meier analysis, Gleason score, Log-rank (Mantel-Cox) test, and Cox hazard analysis. In some aspects, administration of disclosed bispecific antibody, multi-specific antibody, or hybrid antibody in the subject enhances survival, by at least 10%, e.g., 20%, 30%, 40%, 50%, 60%, 70%, 80%, 90%, 95% or above, as compared to survival of a control subject not receiving the treatment or expected survival of the subject before the treatment.

In some aspects, treatment with the disclosed bispecific antibody, multi-specific antibody, or hybrid antibody enhances the death of a malignant cell. Tumor cell death may be determined using any known methods in the art for measuring cell apoptosis or cell viability, including bioluminescent imaging or flow cytometry. In some aspects, administration of disclosed bispecific antibody, multi-specific antibody, or hybrid antibody enhances death of malignant cells in the subject, by at least 10%, e.g., 20%, 30%, 40%, 50%, 60%, 70%, 80%, 90%, 95% or above, as compared to malignant cells, before administration of the inhibitor (baseline) or a control subject not receiving the treatment.

In some aspects, the CAR-immune effector cell therapy or antibody therapy may be accompanied by other therapies, including but not limited to immunotherapy, chemotherapy or radiation therapy. Such therapies can be administered before, simultaneously, or following the administration of CAR-immune effector cells or antibodies. In some aspects, the disclosed CAR-immune effector cells and/or antibodies are administered to a subject in conjunction with any relevant treatments, including but not limited to thalidomide, dexamethasone, bortezomib, and lenalidomide. In further aspects, the CAR-immune effector cells may be used in combination with immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some aspects, the CAR-immune effector cells are administered to a subject in conjunction with bone marrow transplantation, T cell ablative therapy using either chemotherapy agents such as, fludarabine, external-beam radiation therapy (XRT), cyclophosphamide, or antibodies such as OKT3 or CAMPATH. In another aspects, the CAR-immune effector cells of the current disclosure are administered following B-cell ablative therapy such as agents that react with CD20, e.g., Rituxan. In some aspects, subjects may undergo standard treatment with high dose chemotherapy followed by peripheral blood stem cell transplantation. In certain aspects, following the transplant, subjects receive an infusion of the disclosed CAR-immune effector cells. In another aspect, CAR-immune effector cells are administered to the subject before or following surgery.

Any mammal can be treated by the CAR-immune effector cells and/or antibodies described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain aspects, a mammal is a human. In certain aspects a mammal is a non-rodent mammal (e.g., human, pig, goat, sheep, horse, dog, or the like). In certain aspects, a non-rodent mammal is a human. A mammal can be any age or at any stage of development (e.g., an adult, teen, child, infant, or a mammal in utero). A mammal can be male or female. In certain aspects a mammal can be an animal disease model.

### V. Compositions

In some aspects, the disclosure provides compositions comprising disclosed a CAR-immune effector cell, or a population of CAR-immune effector cells. In some aspects, the composition can be formulated as a pharmaceutical composition. In some aspects the pharmaceutical composition comprises a vector, a cell, or a population of cells all as defined earlier herein, preferably for use as a medicament. In some aspects, pharmaceutical compositions comprise CAR-immune effector cells disclosed herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In certain aspects, compositions of CAR-immune effector cells can further comprise other components such as IL-2, IL-15, IL-7, other cytokines or cell populations, or components that sustain the viability and/or activity of administered CAR-immune effector cells may be included in the composition. The CAR-immune effector cells may be formulated as a single dosage unit or as multiple dosage units.

The compositions comprising CAR-immune effector cells disclosed herein, may be formulated for administration, in any convenient manner, including by compositions for injection, transfusion, or implantation. The compositions described herein may be formulated for subcutaneous, intradermal, intratumoral, intranodal, intramedullar, intramuscular, intravenous (i.v.), or intraperitoneal, injection or administration. In some aspects, the disclosed compositions are formulated to be administered by intradermal or subcutaneous injection. In some aspects, the disclosed compositions are formulated to be administered by i.v. injection. The compositions may also be formulated, to be injected directly into a tumor, lymph node, or site of infection. In some aspects, the compositions disclosed herein are formulated for intravenous administration.

In some aspects, the composition can comprise from about 1×10⁴ to 5×10⁹ disclosed CAR-immune effector cells. In some aspects, the composition comprises 1×10⁴, 1×10⁵, 1×10⁶, 1×10⁷, 1×10⁸, 1×10⁹, 2×10⁴, 2×10⁵, 2×10⁶, 2×10⁷, 2×10⁸, 2×10⁹, 3×10⁴, 3×10⁵, 3×10⁶, 3×10⁷, 3×10⁸, 3×10⁹, 4×10⁴, 4×10⁵, 4×10⁶, 4×10⁷, 4×10⁸, 4×10⁹, 5×10⁴, 5×10⁵, 5×10⁶, 5×10⁷, 5×10⁸, or 5×10⁹ of the disclosed CAR-immune effector cells.

In some aspects, disclosed composition comprises a CAR-immune effector cell, wherein the CAR comprises an anti-CD24 antigen recognition domain, comprising a scFV with a nucleic acid with at least about 80% (e.g., about 85%, about 90%, about 95%, about 98%) identity to the sequence of SEQ ID NO: 7 or a scFV with an amino acid with at least about 80% (e.g., about 85%, about 90%, about 95%, about 98%) identity to the sequence of SEQ ID NO: 8.

In some aspects, disclosed composition comprises a CAR-immune effector cell, wherein the CAR comprises an anti-CD24 antigen recognition domain, comprising a scFV with a nucleic acid with the sequence of SEQ ID NO: 7 or a scFV with an amino acid with the sequence of SEQ ID NO: 8.

In some aspects, disclosed composition comprises a CAR-immune effector cell, wherein the CAR is a tandem CAR comprising anti-CD24 antigen recognition domain, comprising a scFV with a nucleic acid with at least about 80% (e.g., about 85%, about 90%, about 95%, about 98%) identity to the sequence of SEQ ID NO: 7 or a scFV with an amino acid with at least about 80% (e.g., about 85%, about 90%, about 95%, about 98%) identity to the sequence of SEQ ID NO: 8, and an anti-BCMA antigen recognition domain comprising a scFV with a nucleic acid with at least about 80% (e.g., about 85%, about 90%, about 95%, about 98%) identity to the sequence of SEQ ID NO: 16 or a scFV with an amino acid with at least about 80% (e.g., about 85%, about 90%, about 95%, about 98%) identity to the sequence of SEQ ID NO: 17.

In some aspects, disclosed composition comprises a CAR-immune effector cell, wherein the CAR is a tandem CAR comprising anti-CD24 antigen recognition domain, comprising a scFV with a nucleic acid with the sequence of SEQ ID NO: 7 or a scFV with an amino acid with the sequence of SEQ ID NO: 8, and an anti-BCMA antigen recognition domain comprising a scFV with a nucleic acid the sequence of SEQ ID NO: 16 or a scFV with an amino acid with the sequence of SEQ ID NO: 17.

In some aspects, disclosed composition comprises a CAR-immune effector cell, wherein the CAR is a bispecific CAR comprising anti-CD24 antigen recognition domain, comprising a scFV with a nucleic acid with at least about 80% (e.g., about 85%, about 90%, about 95%, about 98%) identity to the sequence of SEQ ID NO: 7 or a scFV with an amino acid with at least about 80% (e.g., about 85%, about 90%, about 95%, about 98%) identity to the sequence of SEQ ID NO: 8, and an anti-BCMA antigen recognition domain comprising a scFV with a nucleic acid with at least about 80% (e.g., about 85%, about 90%, about 95%, about 98%) identity to the sequence of SEQ ID NO: 16 or a scFV with an amino acid with at least about 80% (e.g., about 85%, about 90%, about 95%, about 98%) identity to the sequence of SEQ ID NO: 17.

In some aspects, disclosed composition comprises a CAR-immune effector cell, wherein the CAR is a bispecific CAR comprising anti-CD24 antigen recognition domain, comprising a scFV with a nucleic acid with the sequence of SEQ ID NO: 7 or a scFV with an amino acid with the sequence of SEQ ID NO: 8, and an anti-BCMA antigen recognition domain comprising a scFV with a nucleic acid the sequence of SEQ ID NO: 16 or a scFV with an amino acid with the sequence of SEQ ID NO: 17.

In further aspects, the disclosed composition comprises a bispecific antibody, a multi-specific antibody, or a hybrid antibody comprising a first antigen recognition domain comprising an anti-CD24 scFv, as disclosed herein.

### Pharmaceutically Acceptable Carriers, Excipients and Active Agents

In some aspects, the compositions, compositions disclosed herein may further compromise one or more pharmaceutically acceptable diluent(s), excipient(s), and/or carrier(s). Pharmaceutically acceptable diluents, carriers, and excipients can include, but are not limited to, physiological saline, Ringer's solution, phosphate solution or buffer, buffered saline, and other carriers known in the art. Pharmaceutically acceptable carriers include any and all solvents, adjuvants, dispersion media, coatings, surfactants, antioxidants, preservatives (e.g., antibacterial agents, antifungal agents), isotonic agents, absorption delaying agents, salts, drugs, drug stabilizers, gels, binders, excipients, disintegration agents, lubricants, sweetening agents, flavoring agents, dyes, colorants, other medicinal or pharmaceutical agents, wetting agents, emulsifying agents, solution promoters, solubilizers, antifoaming agents, and such like materials and any combinations thereof, as would be known to one of ordinary skill in the art. (See, e.g., Remington's Pharma. Sci. 18th ed. 1990). Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may also be found for example in Remington's Pharma. Sci. 18th ed. 1990. Except insofar as any conventional carrier is incompatible with the active ingredient, its use in the therapeutic or pharmaceutical compositions is contemplated. In certain embodiments, a pharmaceutical composition described herein comprising a population of cells described herein, further comprises a suitable amount of an antifungal agent. In some cases, a pharmaceutical composition described herein comprises an antifungal agent in an amount sufficient for the pharmaceutical composition to retain at least 20%, 30%, 40%, 50%, 60%, 70%, 80% or 90% of its desired activity for a period of at least 1 month, 2 months, 3 months, 6 months, 1 year, 1.5 years, 2 years, 2.5 years or 3 years.

In certain aspects, pharmaceutical compositions described herein may be formulated in conventional manner using one or more physiologically acceptable carriers comprising excipients and auxiliaries to facilitate processing of engineered cells or vectors into preparations which can be used pharmaceutically. In some aspects, any of the well-known techniques, carriers, and excipients may be used as suitable and/or as understood in the art.

In certain aspects, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some aspects, pharmaceutical compositions disclosed herein may comprise at least 5%, at least 10%, at least 20%, at least 25%, at least 30%, at least 35%, at least 40%, at least 45%, or at least 50% total amount of polymers as suspending agent(s) by total weight of the composition. In some aspects, pharmaceutical compositions disclosed herein may comprise about 5% to about 99%, about 10%, about 95%, or about 15% to about 90% total amount of polymers as suspending agent(s) by total weight of the composition.

In certain aspects, pharmaceutical compositions disclosed herein may comprise a viscous formulation. In some aspects, viscosity of composition herein may be increased by the addition of one or more gelling or thickening agents. In some aspects, compositions disclosed herein may comprise one or more gelling or thickening agents in an amount to provide a sufficiently viscous formulation to remain on treated tissue. In some aspects, pharmaceutical compositions disclosed herein may comprise at least 5%, at least 10%, at least 20%, at least 25%, at least 30%, at least 35%, at least 40%, at least 45%, or at least 50% total amount of gelling or thickening agent(s) by total weight of the composition. In some aspects, pharmaceutical compositions disclosed herein may comprise about 5% to about 99%, about 10%, about 95%, or about 15% to about 90% total amount of gelling or thickening agent(s) by total weight of the composition. In some aspects, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In certain aspects, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, or at least 50% total amount of one or more agents by total weight of the composition. In some aspects, pharmaceutical compositions disclosed herein may comprise about 5% to about 99%, about 10%, about 95%, or about 15% to about 90% total amount of one or more agents by total weight of the composition. In some aspects, one or more of these agents may be added to improve the performance, efficacy, safety, shelf-life and/or other property of the muscarinic antagonist composition of the present disclosure. In some aspects, additives may be biocompatible, without being harsh, abrasive, and/or allergenic.

In certain aspects, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In some aspects, pharmaceutical compositions disclosed herein may comprise at least 5%, at least 10%, at least 20%, at least 25%, at least 30%, at least 35%, at least 40%, at least 45%, at least 50% total amount of one or more acidifying agents by total weight of the composition. In some aspects, pharmaceutical compositions disclosed herein may comprise about 5% to about 99%, about 10%, about 95%, or about 15% to about 90% total amount of one or more acidifying agents by total weight of the composition.

In certain aspects, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more alkalizing agents by total weight of the composition. In some aspects, pharmaceutical compositions disclosed herein may comprise about 5% to about 99%, about 10%, about 95%, or about 15% to about 90% total amount of one or more alkalizing agents by total weight of the composition.

In certain aspects, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite and other materials known to one of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more antioxidants by total weight of the composition. In some aspects, pharmaceutical compositions disclosed herein may comprise about 5% to about 99%, about 10%, about 95%, or about 15% to about 90% total amount of one or more antioxidants by total weight of the composition.

In certain aspects, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more buffering agents by total weight of the composition. In some aspects, pharmaceutical compositions disclosed herein may comprise about 5% to about 99%, about 10%, about 95%, or about 15% to about 90% total amount of one or more buffering agents by total weight of the composition.

In some aspects, the amount of one or more buffering agents may depend on the desired pH level of a composition. In some aspects, pharmaceutical compositions disclosed herein may have a pH of about 6 to about 9. In some aspects, pharmaceutical compositions disclosed herein may have a pH greater than about 8, greater than about 7.5, greater than about 7, greater than about 6.5, or greater than about 6.

In certain aspects, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition. In some aspects, pharmaceutical compositions disclosed herein may comprise about 5% to about 99%, about 10%, about 95%, or about 15% to about 90% total amount of one or more preservatives by total weight of the composition.

In certain aspects, pharmaceutical compositions disclosed herein may comprise one or more surface-acting reagents or detergents. In some aspects, surface-acting reagents or detergents may be synthetic, natural, or semi-synthetic. In some aspects, compositions disclosed herein may comprise anionic detergents, cationic detergents, zwitterionic detergents, ampholytic detergents, amphoteric detergents, nonionic detergents having a steroid skeleton, or a combination thereof. In some aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition. In some aspects, pharmaceutical compositions disclosed herein may comprise about 5% to about 99%, about 10%, about 95%, or about 15% to about 90% total amount of one or more surface-acting reagents or detergents by total weight of the composition.

In certain aspects, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition. In some aspects, pharmaceutical compositions disclosed herein may comprise about 5% to about 99%, about 10%, about 95%, or about 15% to about 90% total amount of one or more stabilizers by total weight of the composition.

In some aspects, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some aspects, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some aspects, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition. In some aspects, pharmaceutical compositions disclosed herein may comprise about 5% to about 99%, about 10%, about 95%, or about 15% to about 90% total amount of one or more tonicity modifiers by total weight of the composition.

In some aspect, the pharmaceutical composition may comprise one or more active agents in addition to the CAR, vectors, cells and/or populations of cells provided herein. Non limiting examples of additional active agents include but are not restricted to antibiotics, anti-pyrectics, antimicrobials, antifungals, NSAIDs, chemotherapeutic and anticancer agents.

Chemotherapeutic and anticancer agents commonly used to treat ovarian cancer, and which may be used in combination with any of the compositions disclosed herein include but are not limited to platinum compounds (such as cisplatin or carboplatin), and taxane compounds such as paclitaxel (Taxol®) or docetaxel (Taxotere®). Other chemotherapeutic agents used to treat ovarian cancer and which may be used in combination with any of the compositions disclosed herein, include but are not limited to: Albumin bound paclitaxel (nab-paclitaxel, Abraxane®), Pemetrexed (Alimta®), Irinotecan (CPT-11, Camptosar®), Cyclophosphamide (Cytoxan®), Liposomal doxorubicin (Doxil®), Gemcitabine (Gemzar®), Altretamine (Hexalen®), Ifosfamide (Ifex®), Melphalan (Alkeran), Vinorelbine (Navelbine®), Topotecan (Hycamtin), Etoposide (VP-16), and Capecitabine (Xeloda®).

In some aspects, administration of the composition comprising the disclosed CAR-immune effector cells reduces tumor burden in a subject. In some aspects, reduction in tumor burden in a subject, comprises reduction in number of malignant cells, reduction in tumor size, and/or reduction in the tumor growth. Tumor burden in a subject can be assessed using any conventional methods know in the art, non-limiting examples of which include, diagnostic techniques such as X ray, CT scan, or MRI. In some aspects, administration of disclosed composition reduces number of malignant cells, tumor size, and/or tumor growth in the subject, by at least 10%, e.g., 20%, 30%, 40%, 50%, 60%, 70%, 80%, 90%, 95% or above, as compared to number of malignant cells, tumor size, and/or tumor growth before administration of the inhibitor (baseline) or a control subject not receiving the treatment.

In some aspects, administration of the composition comprising the disclosed CAR-immune effector cells enhances survival and prolongs life of a subject. Survival of the subject can be determined using known methods in the art, non-limiting examples of which include Kaplan Meier analysis, Gleason score, Log-rank (Mantel-Cox) test, and Cox hazard analysis. In some aspects, administration of disclosed composition in the subject enhances survival, by at least 10%, e.g., 20%, 30%, 40%, 50%, 60%, 70%, 80%, 90%, 95% or above, as compared to survival of a control subject not receiving the treatment or expected survival of the subject before the treatment.

In some aspects, administration of the composition comprising the disclosed CAR-immune effector cells enhances death of a malignant cell. Tumor cell death may be determined using any known methods in the art for measuring cell apoptosis or cell viability, including bioluminescent imaging or flow cytometry. In some aspects, administration of disclosed composition enhances death of malignant cells in the subject, by at least 10%, e.g., 20%, 30%, 40%, 50%, 60%, 70%, 80%, 90%, 95% or above, as compared to malignant cells, before administration of the inhibitor (baseline) or a control subject not receiving the treatment.

In further aspects, administration of the composition comprising the disclosed CAR-immune effector cells treats a refractory or relapsed B cell or plasma cell malignancy.

In some aspects, administration of the composition comprising bispecific antibody, multi-specific antibody, or hybrid antibody described herein, can reduces tumor burden in a subject. In some aspects, reduction in tumor burden in a subject, comprises reduction in number of malignant cells, reduction in tumor size, and/or reduction in the tumor growth. Tumor burden in a subject can be assessed using any conventional methods known in the art, non-limiting examples of which include, diagnostic techniques such as X ray, CT scan, bone marrow biopsy, serum protein electrophoresis (SPEP), or MRI. In some aspects, administration of disclosed composition comprising bispecific antibody, a multi-specific antibody, or a hybrid antibody reduces number of malignant cells, tumor size, and/or tumor growth in the subject, by at least 10%, e.g., 20%, 30%, 40%, 50%, 60%, 70%, 80%, 90%, 95% or above, as compared to number of malignant cells, tumor size, and/or tumor growth before administration of the inhibitor (baseline) or a control subject not receiving the treatment.

In some aspects, administration of the composition comprising bispecific antibody, multi-specific antibody, or hybrid antibody disclosed herein enhances survival and prolongs life of a subject. Survival of the subject can be determined using known methods in the art, non-limiting examples of which include Kaplan Meier analysis, Gleason score, Log-rank (Mantel-Cox) test, and Cox hazard analysis. In some aspects, administration of disclosed composition comprising the bispecific antibody, multi-specific antibody, or hybrid antibody in the subject enhances survival, by at least 10%, e.g., 20%, 30%, 40%, 50%, 60%, 70%, 80%, 90%, 95% or above, as compared to survival of a control subject not receiving the treatment or expected survival of the subject before the treatment.

In some aspects, administration of the composition comprising bispecific antibody, multi-specific antibody, or hybrid antibody disclosed herein, enhances the death of a malignant cell. Tumor cell death may be determined using any known methods in the art for measuring cell apoptosis or cell viability, including bioluminescent imaging or flow cytometry. In some aspects, administration of the disclosed composition comprising bispecific antibody, multi-specific antibody, or hybrid antibody enhances death of malignant cells in the subject, by at least 10%, e.g., 20%, 30%, 40%, 50%, 60%, 70%, 80%, 90%, 95% or above, as compared to malignant cells, before administration of the inhibitor (baseline) or a control subject not receiving the treatment.

In further aspects, administration of the composition comprising the disclosed bispecific antibody, multi-specific antibody, or hybrid antibody disclosed herein treats a refractory or relapsed B cell or plasma cell malignancy.

The actual dosage amount of a composition according to the present disclosure, and the selection of any combination treatment to be administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

### Patient Samples and Cell Lines

PBMCs and BMMCs were obtained from University of Arkansas for Medical Sciences (UAMS) Myeloma Center Tissue Biorepository and Procurement Core. The UAMS institutional review board approved these research studies (IRB 261817 and 261821). Cell lines were obtained from ATCC and cultured according to the manufacturer's instructions.

### Construction of CAR Vector

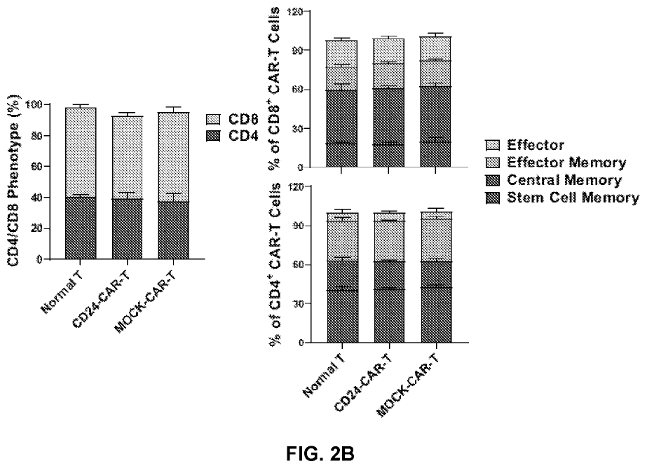

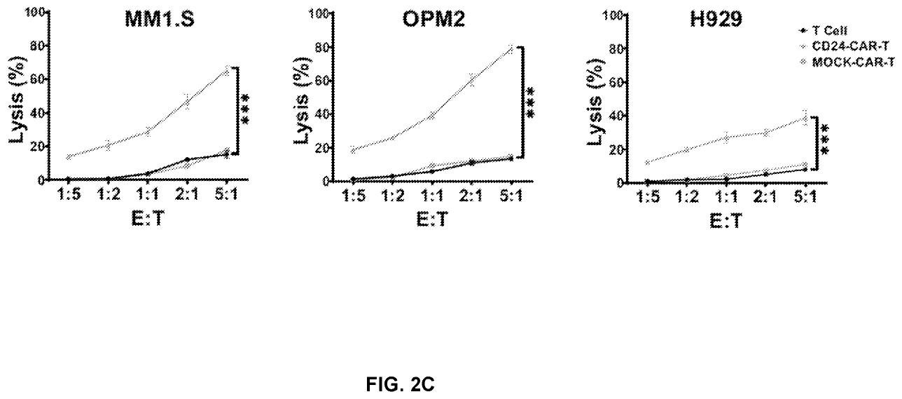

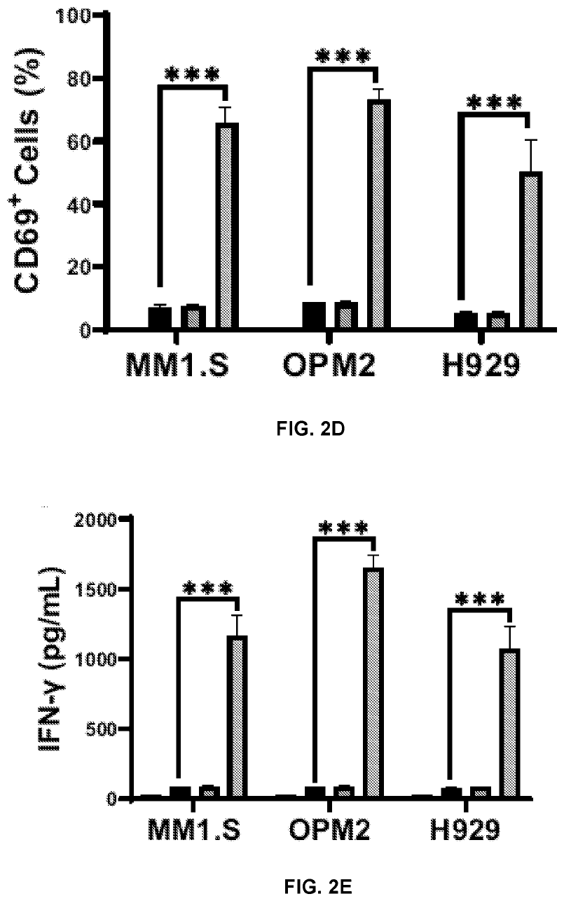

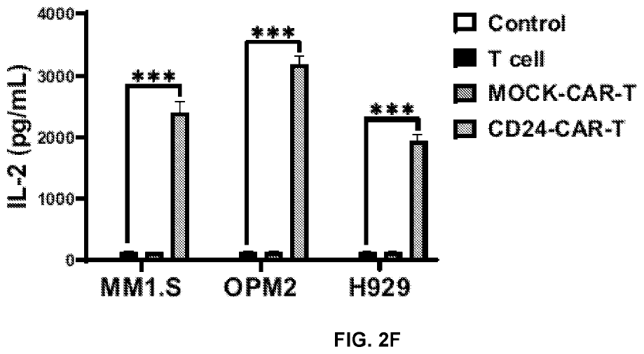

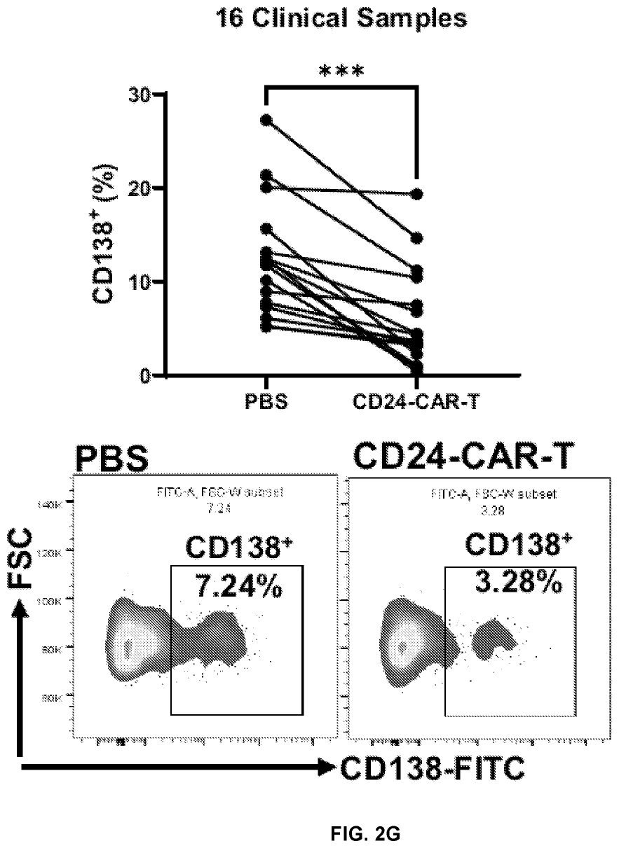

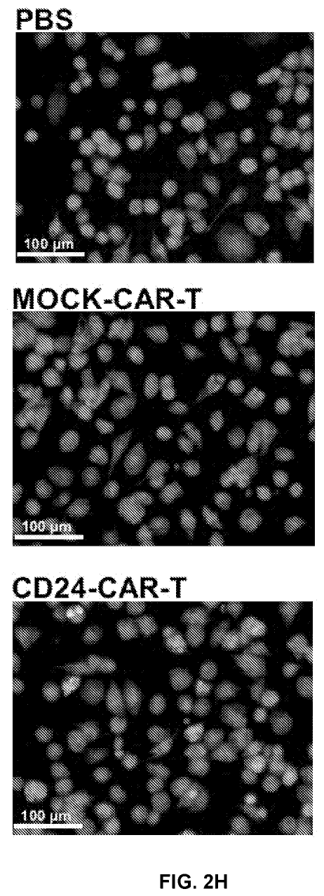

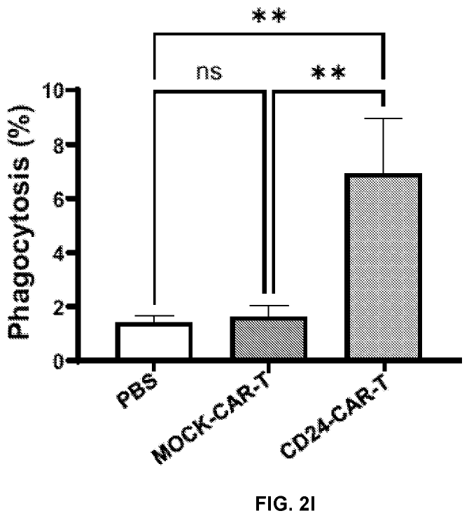

CD24-CAR vector was constructed containing CD24-specific scFvs (derived from SWA11), a safety switch (RQR8) in the hinge region, and a 4-1BB co-activation domain with CD3ζ. The MOCK-CAR vector only contains a safety switch in the hinge region and a 4-1BB co-activation domain with CD3ζ (FIG. 2A). The BCMA-CAR vector contains BCMA-scFv (derived from clone C11D5.3) a safety switch in the hinge region, and a 4-1BB co-activation domain with CD3ζ. The dual-targeted BCMA-CD24

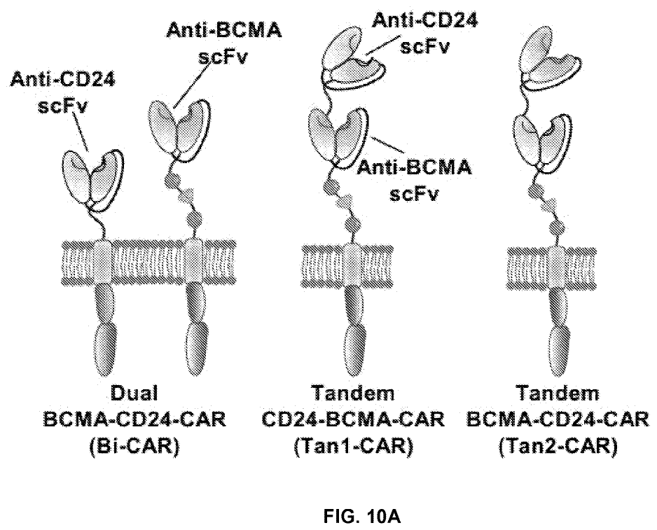

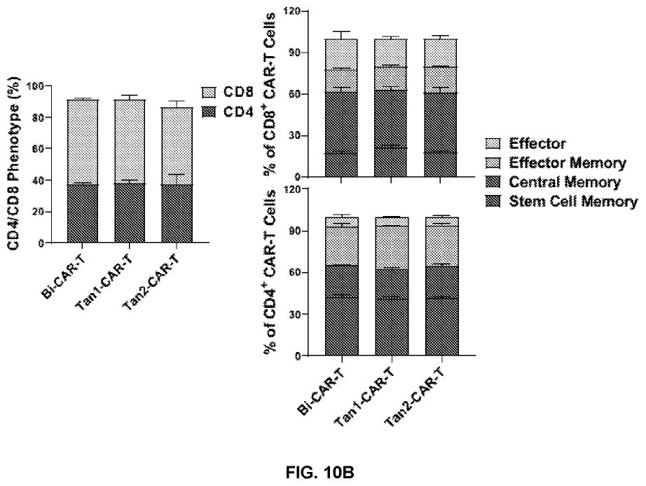

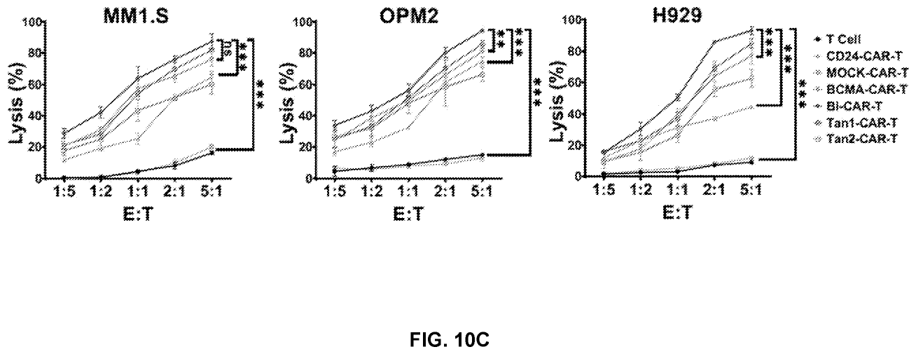

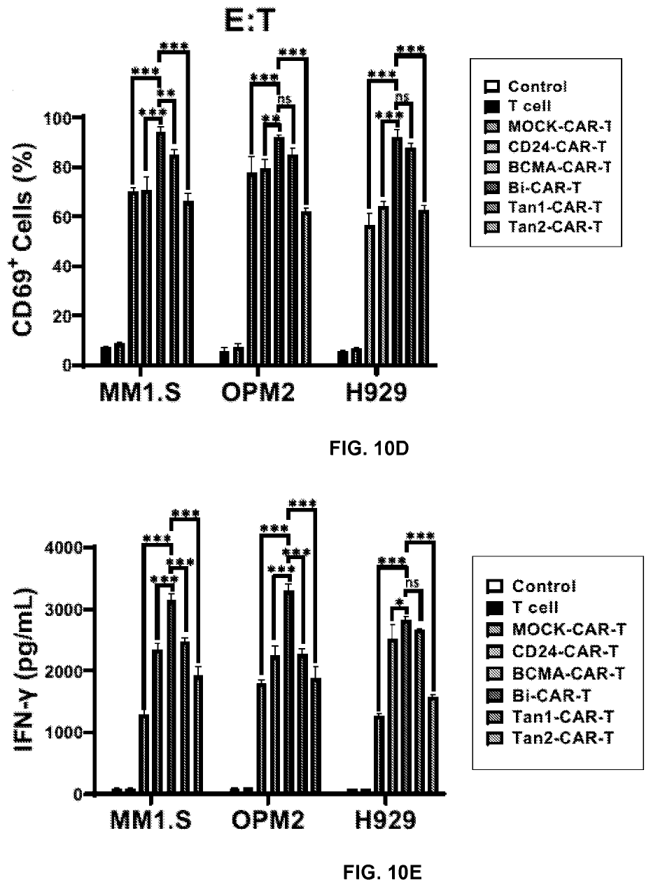

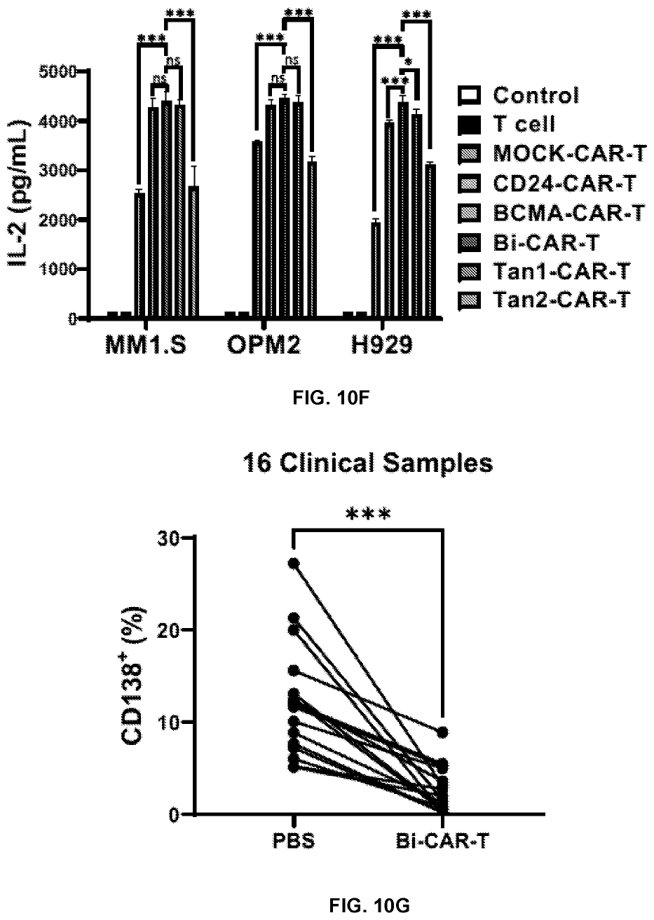

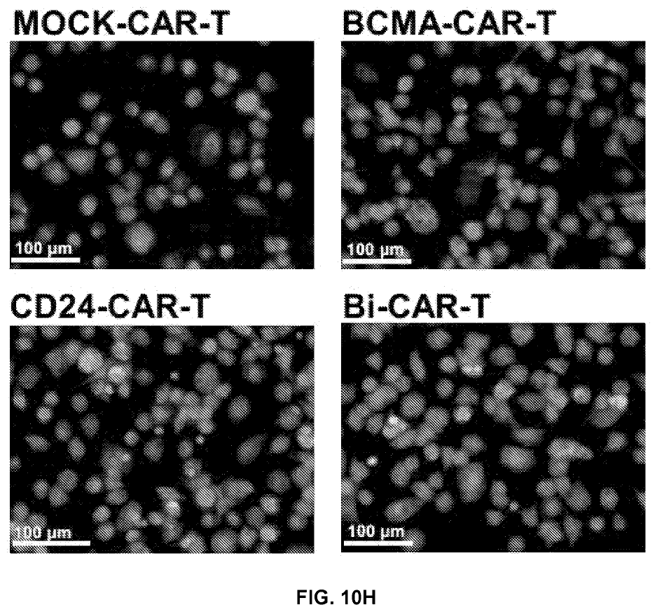

CAR vector with 2 complete CAR units: BCMA-CAR and CD24-CAR. A P2A self-cleaving peptide was inserted between the 2 CAR vectors. Two tandem CAR vectors were constructed by linking CD24-scFv and BCMA-scFv with poly-Glycine-Serine (G4S) 4 peptide linker (FIG. 10A). To decrease the risk of severe immunological side effects, we integrated RQR8, an immunological safety switch, with a CD34 epitope and 2 CD20 mimotopes as a suicide molecule. All vectors contain SFFV promoters to drive the expression of CARs.

### Transfection of T Cells

Lentivirus Density-gradient centrifugation was used to isolate PBMCs from healthy donor samples by Ficoll-Paque (General Electric). T cells were isolated from PBMCs with CD3 Microbeads (Miltenyi) and were cultured in AIM V Medium (Thermo Fisher Scientific) with 5% human AB serum (Sigma-Aldrich) and 400 IU IL-2 (R&D Systems). Dynabeads Human T-activator CD3/CD28 (Thermo Fisher Scientific) were added for T-cell expansion and activation. T cells were activated for 2 to 3 days prior to transduction.

Lentivirus particles were used to transduce T cells. T cells and concentrated lentivirus were added into RetroNectin precoated plates (Takara Bio). Cells were cultured in AIM V Medium for 24 hours, and the transduction step was repeated. After 24 hours, cells were washed with PBS and cultured in the fresh medium for 7 days. CAR-T cells were detected by BD FACSVerse™ flow cytometry with CD34 antibodies (BioLegend) and CD34 Microbeads (Miltenyi) for isolation. Cell Counting Kit-8 (APExBIO) was used to detect CAR-T-cell proliferation. Phenotypes of CAR-T cells were detected by flow cytometry. T cells memory phenotypes were assigned according to CD62L and CD45RO expression within the CAR-T cell population as follows: stem cell memory (CD45RO⁻/CD62L⁺), central memory (CD45RO⁺/CD62L⁺), effector memory (CD45RO⁺/CD62L⁻), effector cells (CD45RO⁻/CD62L⁻).

### Cytotoxicity Assay

The cytolytic activity of CAR-T cells was detected based on fluorescence intensity. In 96-well plates, 20×10³ mCherry-positive MM cells were seeded. CAR-T cells or T cells were added at the effector/target (E/T) ratio from 1:5 to 5:1. Then fluorescence intensity was measured at excitation 585 nm/emission 620 nm with a plate reader (BioTek) after 24 hours of coculture.

$$\% lysis = \frac{experimental lysis - spontaneous lysis}{maximal lysis - spontaneous lysis} \times 100 \%$$

The T-cell activation marker CD69 was detected and measured the cytokines IFN-γ, IL-2, and TNF-α, which are produced by activated CAR-T cells, in the supernatant after 24 hours of coculture.

### Macrophage Phagocytosis

MM.1S cells, BMMCs were cultured in Iscove's Modified Dulbecco's Media (IMDM) medium with 10% human AB serum and macrophage colony-stimulating factor for 7 days to induce macrophage differentiation. IFN-γ and lipopolysaccharide (LPS) were added on day 3.

The mature macrophages were stained with DiD dye before phagocytotic experiments. In vitro phagocytosis was performed by coculture of OPM2 cells that expressed GFP, DiD stained macrophages, and CAR-T cells at a ratio of 2:1:1 for 4 hours in the 37 °C incubator. Next, cells were washed with PBS twice, and GFP⁺DiD⁺ cells were detected by BD FACSVerse™ flow cytometry and ZEISS Axio Observer fluorescence microscope.

### 5TGM1/KaLwRij MM Mouse Models

Eight-week-old female C57BL/KaLwRij mice were injected intravenously via the tail vein with either 100 μL PBS or 1×106 5TGM1-GFP cells stained with DiD dye and randomized into 3 groups (n=5/group). Seven days after the 5TGM1 injections, mice were injected intravenously via the tail vein with either PBS (PBS group), 1×106 MOCK-CAR-T cells (MOCK-CAR T group), or CD24-CAR-T cells (CD24-CAR T group). Twenty-one days after the 5TGM1 injections, the experiment was terminated because the PBS group started to develop paraplegia; the mice were killed. Serum protein electrophoresis (SPE) was performed by QuickGel Electrophoresis (Helena Laboratories). BMMCs were isolated by Ficoll-Paque. Dormant (GFP+DiDHi) and activated (GFP+DiDNeg) cells were detected by flow cytometry. BM microenvironmental cells were sorted out with fluorescence-activated cell sorting by depleting 5TGM1-GFP+ MM cells and human CD34+ CAR-T cells. Sorted cells with a purity greater than 98% were used for scRNA-seq.

### Single-Cell RNA Sequencing

Single-cell emulsions were generated with the Chromium Next GEM Chip G Single Cell Kit (10× Genomics) and the Chromium Next GEM Single Cell 3′ v3.1 Kit (10× Genomics) following the standard protocol. Libraries were assessed for mass concentration with the Qubit 1× dsDNA High Sensitivity Assay Kit (Thermo Fisher Scientific). Library fragment size was assessed with the High Sensitivity NGS Fragment Analysis Kit (Agilent) on the Fragment Analyzer System (Agilent). Libraries were functionally validated with the KAPA Library Quantification Kit (Roche). Initial low-pass “surveillance” sequencing was performed on a NovaSeq SP 100-cycle Flow Cell (Illumina) and data was assessed with the Cell Ranger count (10× Genomics) output.

Bioinformatic Analysis of scRNA-Seq

The raw scRNA-seq data were preprocessed with Cell Ranger v6 (10× Genomics) and reference genome of Mus musculus version mm10 to demultiplex for cell and transcript and generate count table. The count table was loaded into R through the Seurat V4 package for further analysis. The cells that have fewer than 500 genes, greater than 5,000 genes, and more than 10% of unique molecular identifiers stemming from mitochondrial genes were discarded from the analysis. For individual samples, principal component analysis (PCA) was then performed on significantly variable genes of the remaining high-quality cells. The results of the individual samples were used for data integration across samples with the reciprocal PCA method to minimize technical differences between samples. Then the nitration results were used as input for clustering with the Louvain algorithm with multilevel refinement and UMAP. The gene-specific markers of each cluster were identified with the FindMarkersAll function with MAST (Model-based Analysis of Single-cell Transcriptomics) method test statistics. The expression pattern of selected gene markers across cell clusters was plotted as a heat map to assess the quality of cell clustering results. To visualize some of the gene markers simultaneously in kernel joint density estimation, the Nebulosa package was used. Gene-set enrichment analysis of marker genes was performed on KEGG pathway analysis.

### Human MM Cell Lines Xenograft Mouse Models

Eight-week-old NOD.Cg-Prkdcscidll2rgtm1Wjl/SzJ (NSG) mice were administered 1.5×10⁶ MM1.S or OPM2 cells by intravenous injection and randomized into 5 groups (n=5/group). On day 7 after MM1.S or OPM2 cell injection, 1.5×106 CAR-T cells or PBS was administered. Mice were weighed and monitored for signs of distress every 3 days. D-luciferin was intraperitoneally injected with 150 mg/kg, and bioluminescence images were acquired 10 minutes later. Myeloma progression was monitored by bioluminescence images every 7 days until the mice developed hind limb paralysis or the bioluminescence signal was more than 2×10¹⁰.

### MM Patient-Derived Xenograft Mouse Models

Eight-week-old NSG mice were administered with sublethal irradiation (2.5 Gy). After radiation, each mouse was intravenously injected with 5×10⁶ 7.5% CD138+ MM patient-derived BMMCs. SPE was performed every 2 weeks. On day 30, tumor-bearing mice received the 1.5×10⁶ CAR-T cells or PBS. The mice were killed when they were weak or with hind limb paralysis. All animal procedures adhered to a protocol (3997) approved by the local Institutional Animal Care and Use Committee at UAMS.

### Statistical Analysis

All data were analyzed with Prism 9 (GraphPad) and presented as means±standard deviation (SD) unless otherwise indicated. For statistical analyses, a two-tailed Student t-test was performed when only 2 groups were compared. One-way ANOVA analysis of variance was used to determine the statistically significant difference for multiple group comparisons. A p-value of 0.05 or less was considered significant.

### Data and Code Availability

The authors are committed to the open sharing of data. Raw data files for sc RNA-seq data have been deposited in the Gene Expression Omnibus (https://www.ncbi.nlm.nih.gov/geo/, accession # is pending).

### Example 1-CD24 Expression is Increased in MM Cells after Treatment

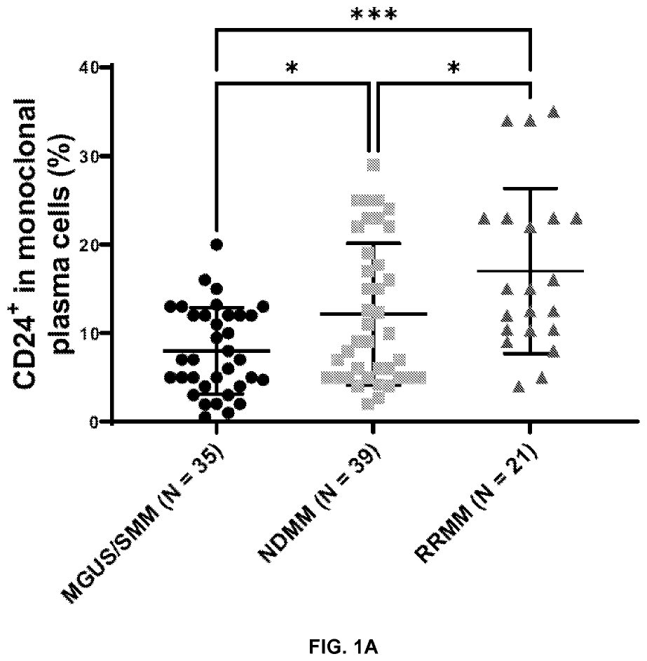

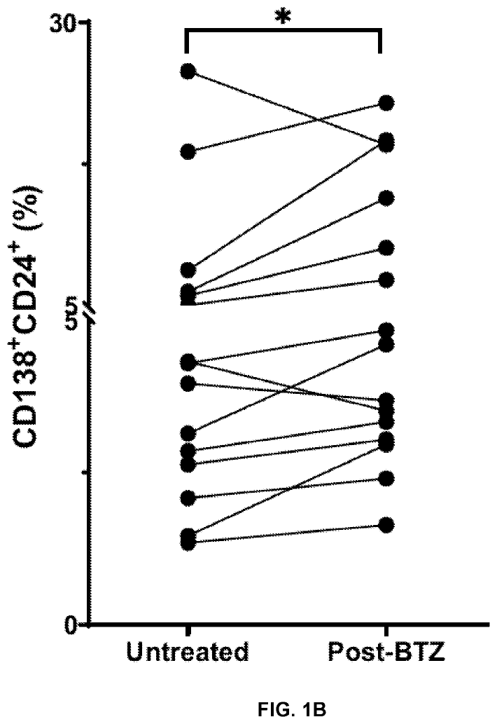

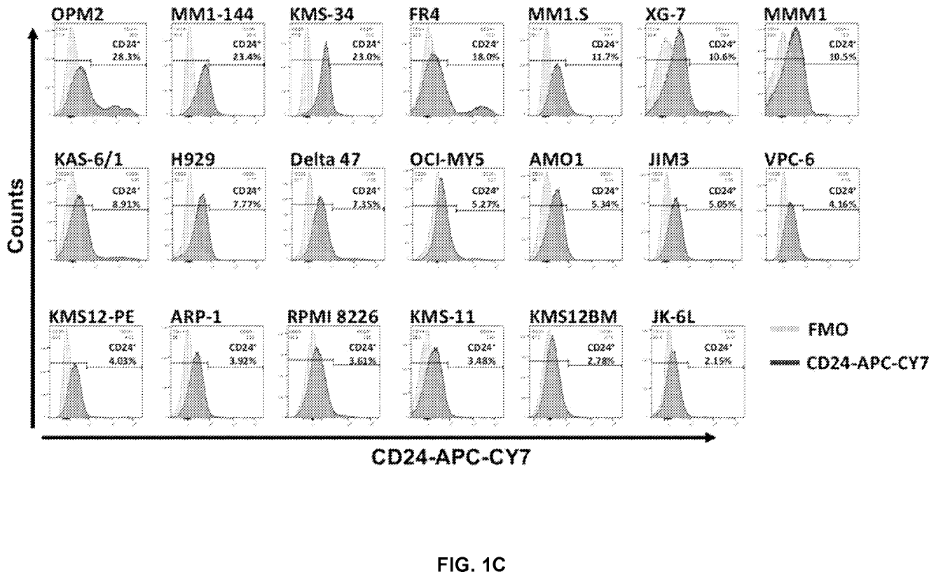

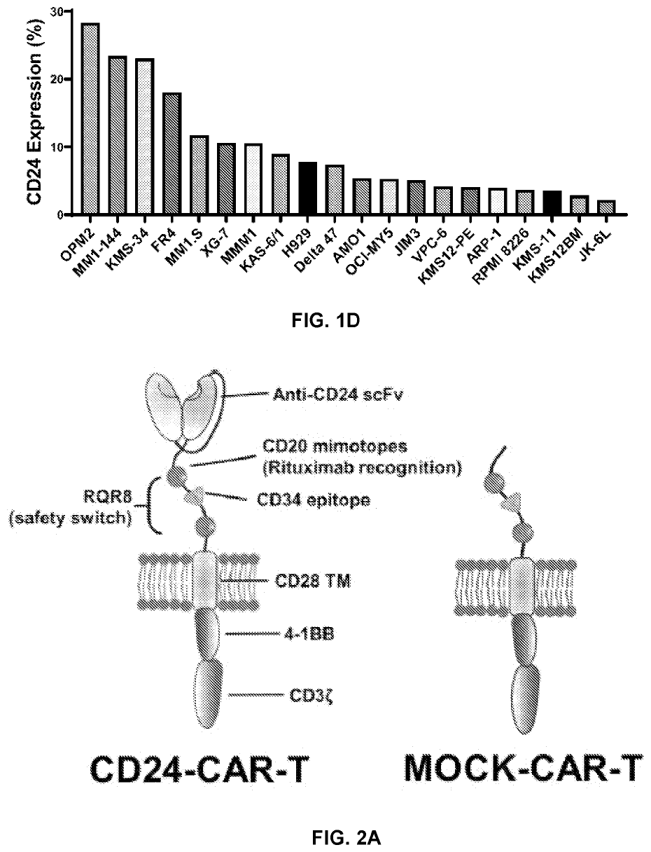

Bone marrow mononuclear cells (BMMCs) were collected from 35 patients with monoclonal gammopathy of undetermined significance (MGUS) and smoldering multiple myeloma (SMM), 39 patients with newly diagnosed MM (NDMM), and 21 patients after treatment with relapsed/refractory MM (RRMM). Using flow cytometry on BMMC, the percentage of CD24⁺/CD138⁺/CD38⁺/CD45⁻ plasma cells was found significantly increased RRMM (17.00%±9.31%) compared with patients with MGUS/SMM (8.00%±4.90%) and NDMM patients (12.14%±8.00%; FIG. 1A). The MM patients' BMMCs were treated with 5 nM bortezomib (BTZ) for 24 hours. The percentage of CD138⁺CD24⁺ cells were found increased in 12 of 15 BMMC samples post-BTZ (FIG. 1B). The results showed the association between drug resistance and CD24 expression. CD24 expression was detected in 20 MM cell lines. It was found that CD24 was broadly expressed in all MM cell lines. The MM cell lines OPM2 (28.3% cells are CD24⁺), MM1.S (11.7% cells are CD24⁺) and H929 (7.77% cells are CD24⁺) were used for the in vivo and in vitro experiments. (FIG. 1C and FIG. 1D).

### Example 2-Construction and Characteristics of CD24-CAR-T Cells

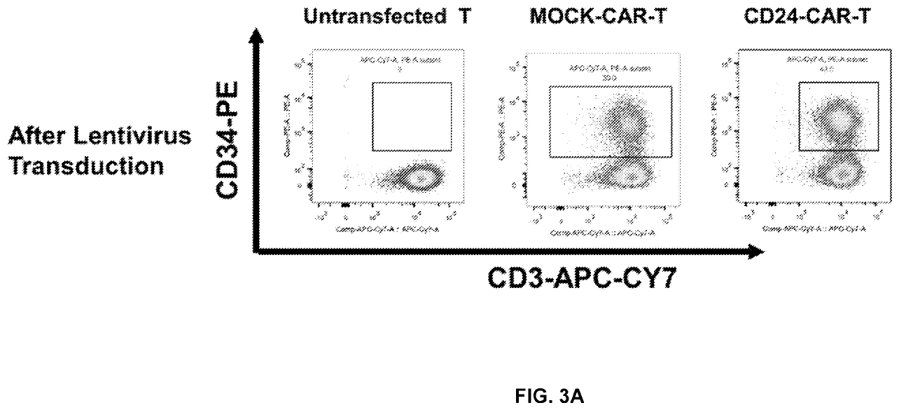

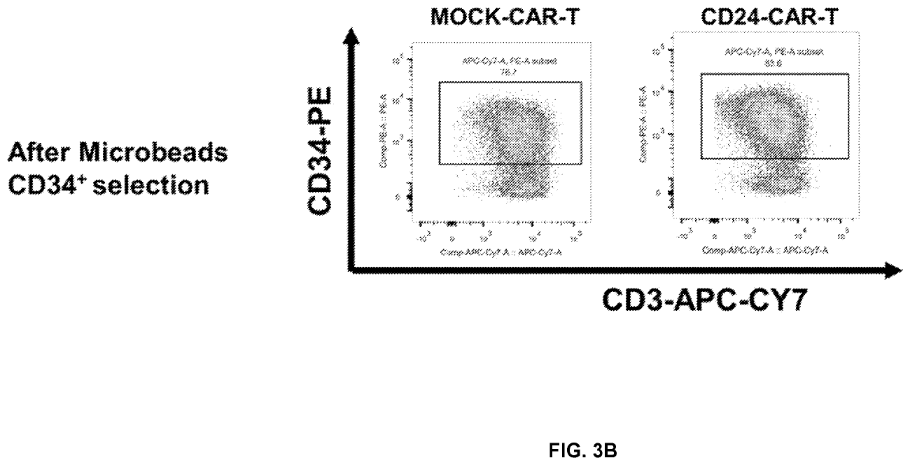

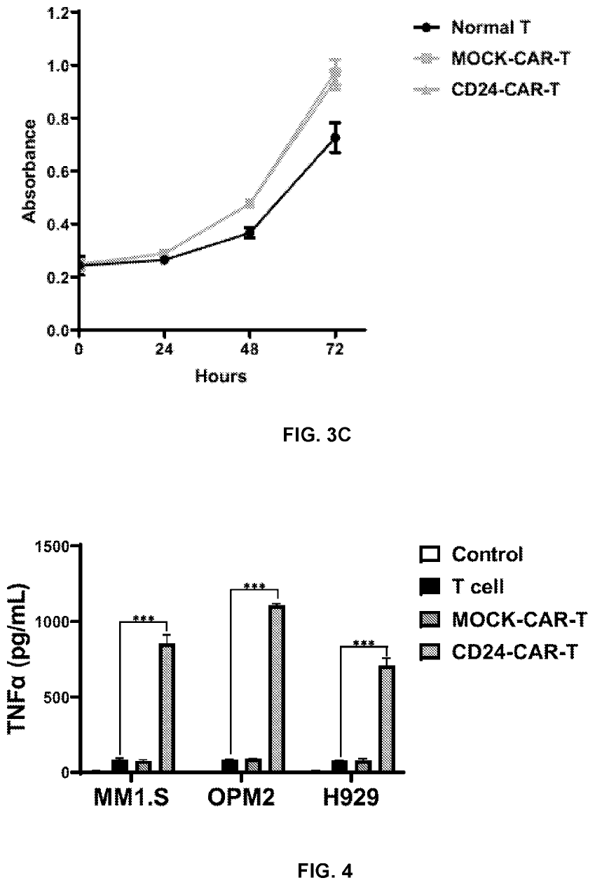

A second-generation, CD24-CAR vector was constructed by CD24-specific single-chain variable fragments (scFvs; derived from anti-CD24 antibody, SWA11), a safety switch (marker-suicide gene, RQR8) in the hinge region, and a 4-1BB co-activation domain with CD3ζ. To decrease the risk of severe immunological side effects, we integrated RQR8 with two CD20 mimotopes as a suicide molecule and one CD34 epitope for detection (FIG. 2A). The CD3⁺ T cells from healthy donors' peripheral blood mononuclear cells (PBMC) were modified. The transfection efficacy of CAR-T cells was detected by flow cytometry. The transfection efficacy of CD24-CAR-T cells was 43% (FIG. 3A). After the CAR-T cells were isolated, the CD34 positive rate reached a high of 83.6% (FIG. 3B). The results of the proliferation assay showed CAR-T cells proliferated well in vitro (FIG. 3C). The phenotype of CAR-T cells, including the ratio of CD4 and CD8 phenotypes and the ratio of different memory phenotypes, was detected by flow cytometry. The mean percentage of CD4 CD24-CAR-T cells was 38.9% and CD8 was 53.9%. The combined percentage of CD4 stem cell memory (41.2%) and central memory (21.5%) was more than 60%. CD8 stem cell memory (17.7%) and central memory (43.6%) were also prominent (FIG. 2B). Previous studies found that early memory phenotypes (stem cell memory and central memory) drive T-cell proliferation, persistence, and antitumor effects.

### Example 3-CD24-CAR-T Cells Successfully Eliminate MM Cells In Vitro

Analysis of coculture-killing assays of CAR-T cells with OPM2, MM1.S, and H929 indicated that effector CAR-T cell-mediated target tumor cytotoxicity increased as the effector: target (E:T) ratio increased in 3 MM cell lines. When the CD24-CAR-T cell: MM ratio was 5:1, the percentages of target cells lysed were 78.9% in OPM2 cell line, and there was a 5.3-fold increase in percent lysis seen with CD24-CAR-T compared to the average of MOCK-CAR-T. The percentages of target cells lysed were 65.0% in MM1.S cell line, and there was a 3.7-fold increase in percent lysis seen with CD24-CAR-T compared to the average of MOCK-CAR-T. The percentages of target cells lysed were 38.7% in H929 cell line, and there was a 3.4-fold increase in percent lysis seen with CD24-CAR-T compared to the average of MOCK-CAR-T (FIG. 2C). CD24-CAR-T cells activation was determined by increased CD69 expression (FIG. 2D). Compared with the MOCK-CAR-T group, interferon (IFN)-γ, interleukin (IL)-2, and tumor necrosis factor (TNF)-α production was dramatically increased in the CD24-CAR-T group (FIG. 2E, FIG. 2F, and FIG. 4). These results suggest that exposure to CD24⁺ MM cells resulted in strong activation of CD24-CAR-T cells. When the efficacy of CD24-CAR-T was tested against primary MM cells, the percentage of the subpopulation of CD138⁺ cells decreased in MM patients' BMMCs samples post-CD24-CAR-T treatment (FIG. 2G). These results showed that primary MM cells were significantly lysed by CD24-CAR-T cells.

### Example 4-CD24-CAR-T Cells Promote Phagocytic Clearance by Macrophages in Vitro

The CD24-CAR-T cells were incubated with DiD (1,1-dioctadecyl-3,3,3,3-tetramethylindodicarbocyanine; red) stained macrophages and OPM2 stably expressing GFP (green fluorescent protein) to detect the phagocytic clearance of MM cells. Phagocytosis events were visualized as green cells inside of the red cells. Flow cytometry detected the DiD+GFP⁺ signals as phagocytosis events. It was found that compared with the PBS and MOCK-CAR-T groups, CD24-CAR-T cells showed a strong increase in macrophage phagocytic clearance (FIG. 2H and FIG. 2I).

### Example 5-CD24-CAR-T-Cell Treatment Reduces MM Burden in the 5TGM1 Mouse Model

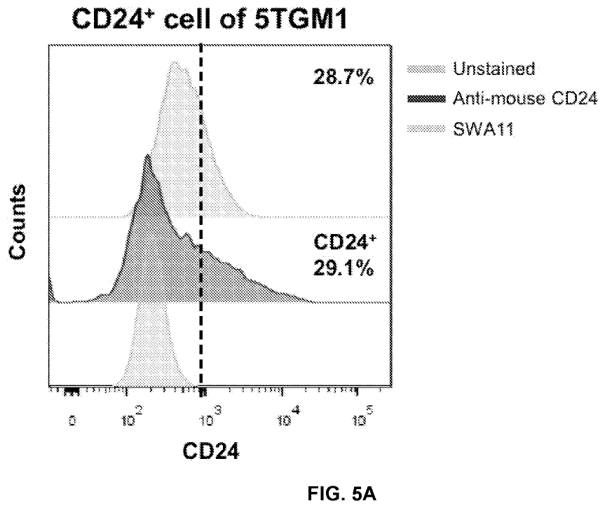

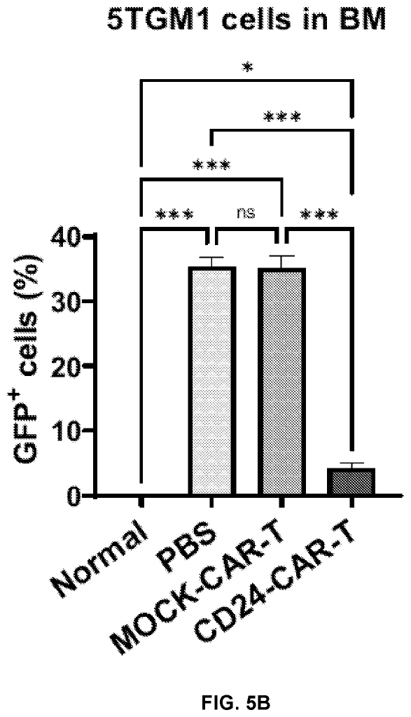

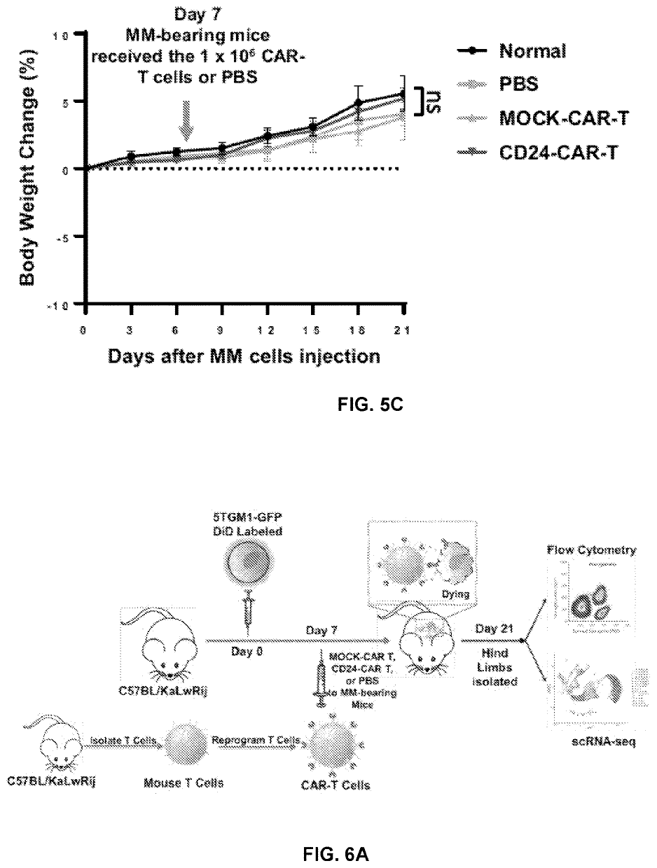

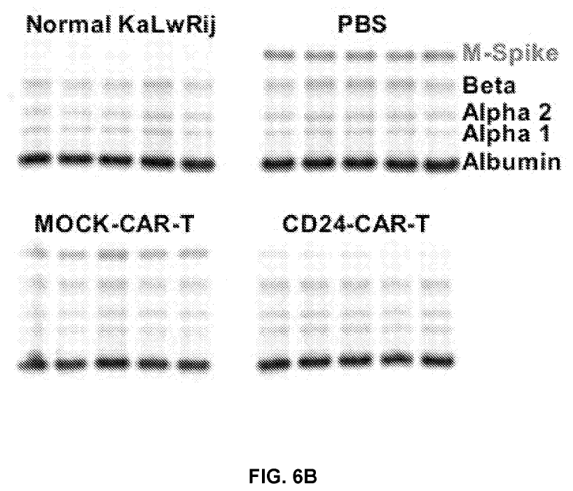

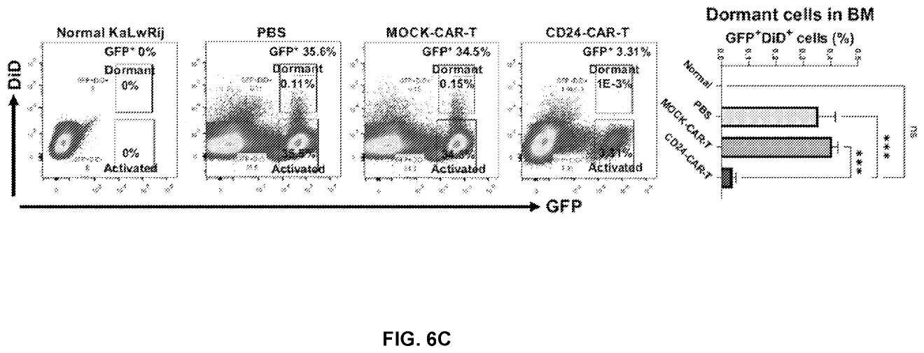

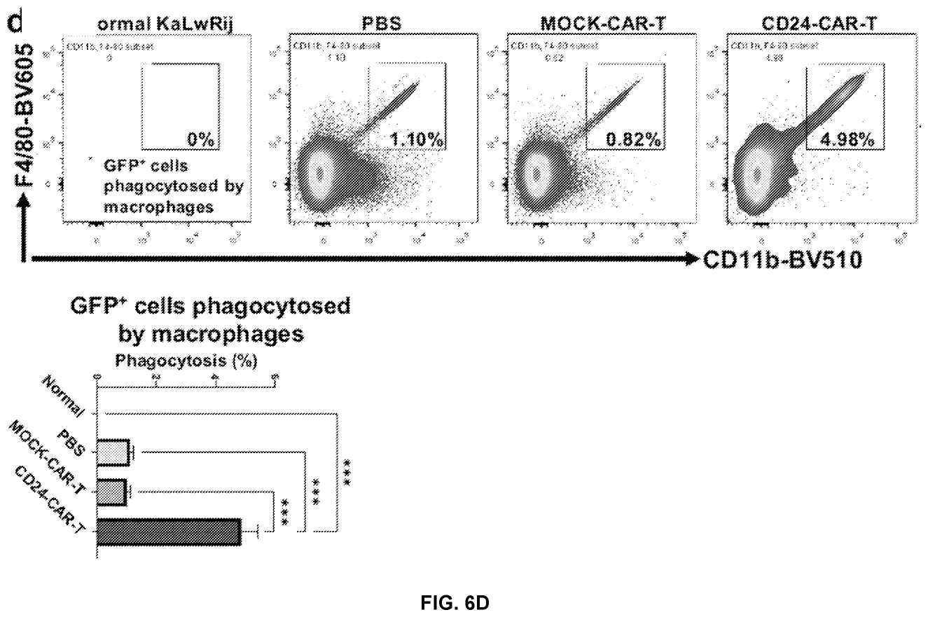

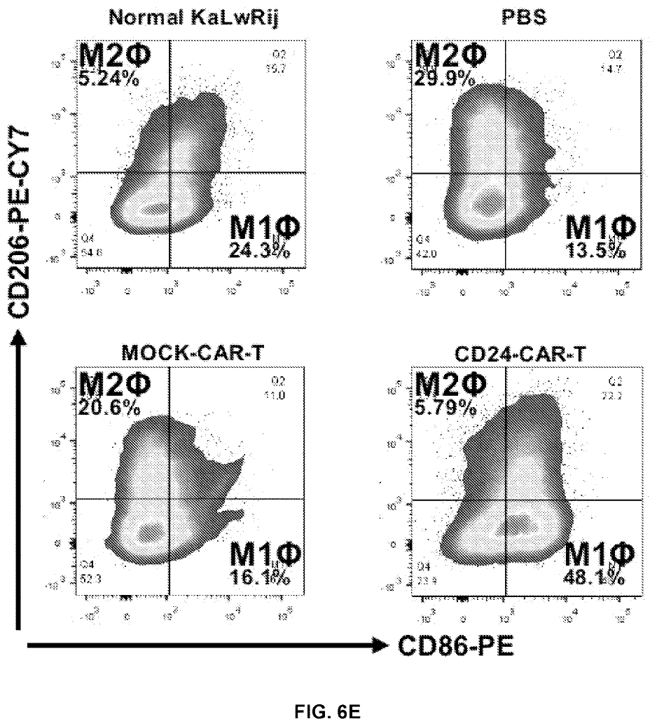

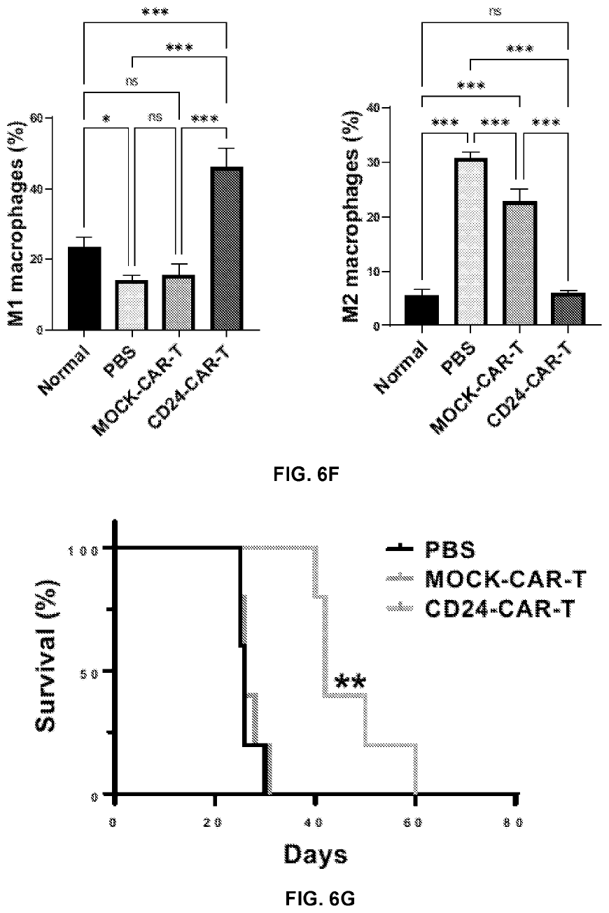

To the assess Flow cytometry results showed that 5TGM1 MM cells express CD24 (29.1%) and that the SWA11 antibody could target CD24 on 5TGM1 cells (FIG. 5A). The CD24-CAR-T cells were tested in the immune-competent 5TGM1/C57BL/KaLwRij mouse model (FIG. 6A). The mice were divided into 4 groups: 1) normal group (healthy mice without 5TGM1 injection), 2) the PBS group (treated with PBS after 5TGM1 injection), 3) MOCK-CAR-T group (treated with MOCK-CAR T after 5TGM1 injection), and 4) CD24-CAR-T group (treated with CD24-CAR T after 5TGM1 injection). Serum protein electrophoresis (SPE) results showed the M protein of the PBS group and the MOCK-CAR-T group increased substantially when compared with the normal group, while the M protein of the CD24-CAR-T group was significantly reduced compared with the PBS group and the MOCK-CAR-T group (FIG. 6B). 5TGM1 cells (GFP⁺ cells) were more than 8-fold lower in the CD24-CAR-T group (4.2%) than in the PBS (35.4%) and MOCK-CAR T (35.0%) groups (FIG. 5B). These results indicated that the CD24-CAR-T reduced tumor burden in the 5TGM1 model.

To detect whether CD24-CAR-T cells can eliminate dormant TICs, 5TGM1-GFP MM cells were stained with DID before injection. Dormant TICs were identified by expression of GFP and retention of DID (GFP⁺DiD^{Hi}). Activated cells that had proliferated and diluted the DiD label were GFP⁺DiD^{Neg}. The results showed that dormant cells (GFP⁺DiD^{Hi}) were significantly decreased in the CD24-CAR-T group compared with the MOCK-CAR-T group (FIG. 6C) indicating that CD24-CAR-T cells depleted 5TGM1 dormant cells in vivo. To determine the percentage of tumor cells phagocytosed by macrophages, flow cytometry was performed and gated on CD11b and F4/80 double-positive macrophages; the phagocytosis ratio was represented by GFP⁺ macrophages because 5TGM1 cells were GFP⁺. It was found that the percentage of GFP⁺ macrophages was significantly higher in the CD24-CAR-T group than that in the PBS and MOCK-CAR-T groups (FIG. 6D). These findings suggested that the CD24-CAR-T cells promoted phagocytic clearance of tumor cells in vivo.

In the macrophage (CD11b⁺F4/80⁺) population, CD86 is used as a marker of M1 macrophages (M1ct), while CD206 is used as the marker of M2 macrophages (M2ct). M1ct is involved in the clearance of tumor cells and the M2ct is an immunosuppressive macrophage. After CD24-CAR-T treatment, a significant change in the ratios of M1ct and M2ct cells in the myeloma microenvironment was observed. Compared with the normal group, M1ct cells were reduced by more than 1.7-fold in the PBS group and the MOCK-CAR-T. Conversely, M2ct cells were increased by greater than 5.5-fold, indicating the ability of tumor cells to drive macrophages toward an M2ct phenotype. After CD24-CAR-T treatment, this phenomenon was reversed. M1ct increased greater than 2-fold compared to the normal group, while M2ct returned to normal levels (FIG. 6E and FIG. 6F). These data showed that tumor cells shift macrophage differentiation to a tumor-permissive phenotype and that CD24-CAR-T reversed this phenomenon.

Importantly, it was also found that the median survival was more than 60% longer in the CD24-CAR-T group (42 days) than in control groups (26 days each; p=. 0018; FIG. 6G), with no significant decrease in mice weight after treatment with CD24-CAR-T cells (FIG. 5C). These data showed that CD24-CAR-T eliminated tumor cells in vivo and improved the outcome of MM-bearing mice.

### Example 6-Single-Cell RNA Sequencing

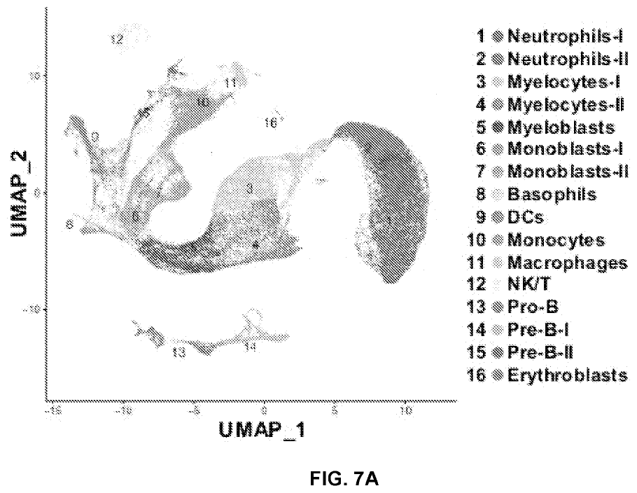

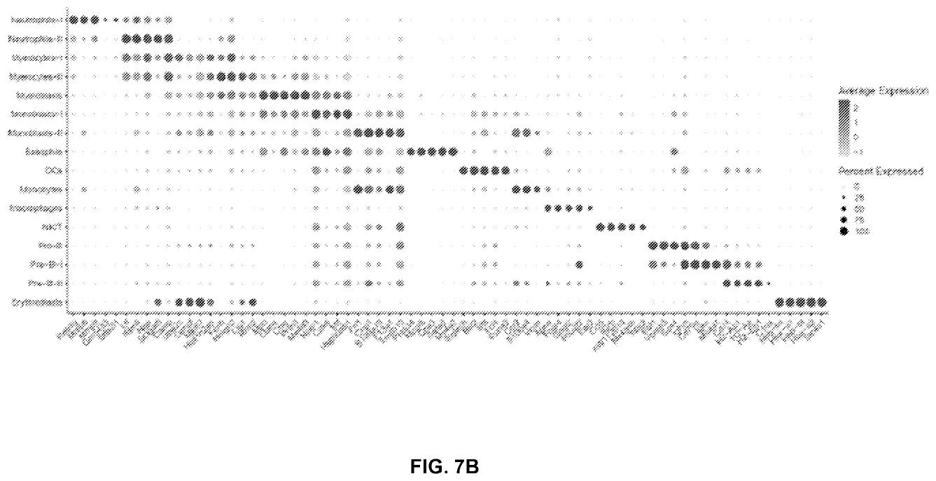

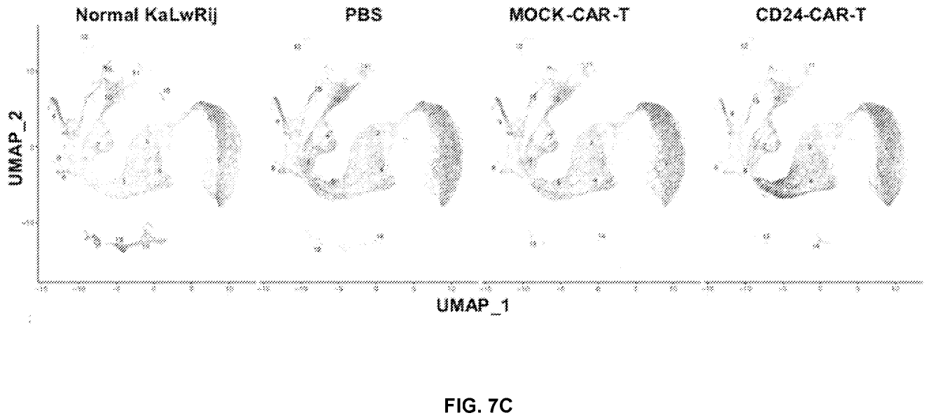

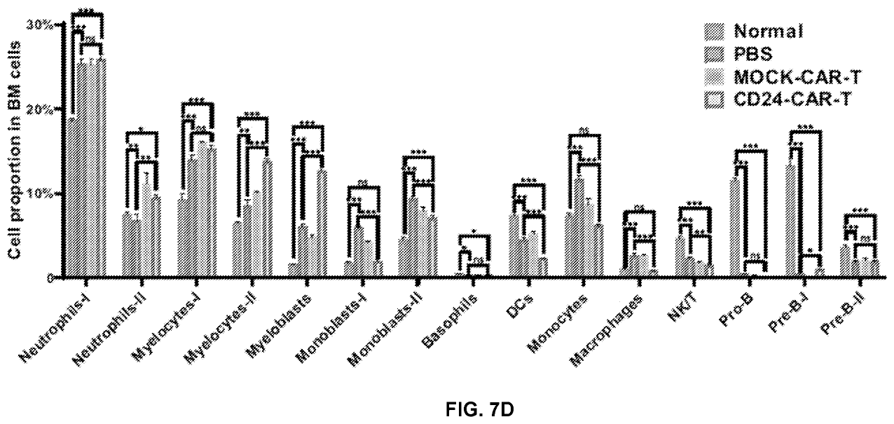

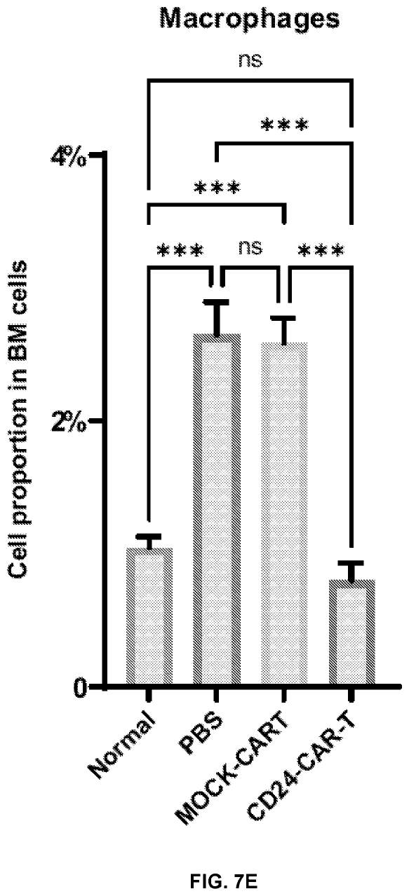

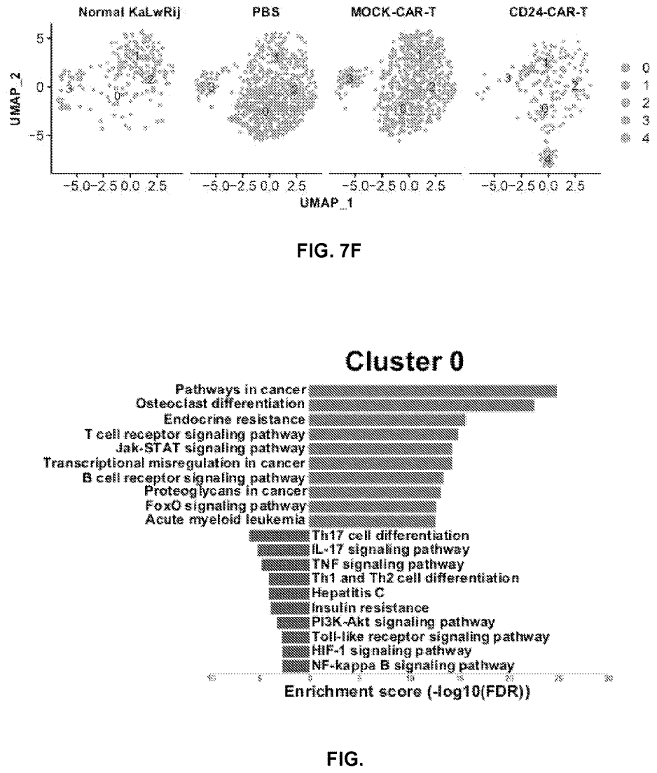

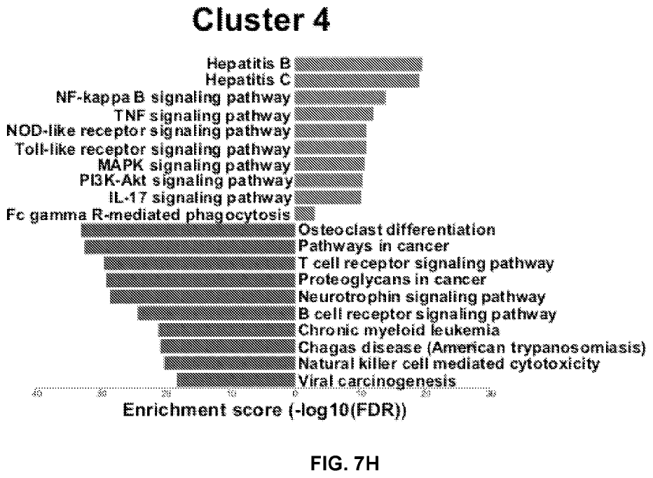

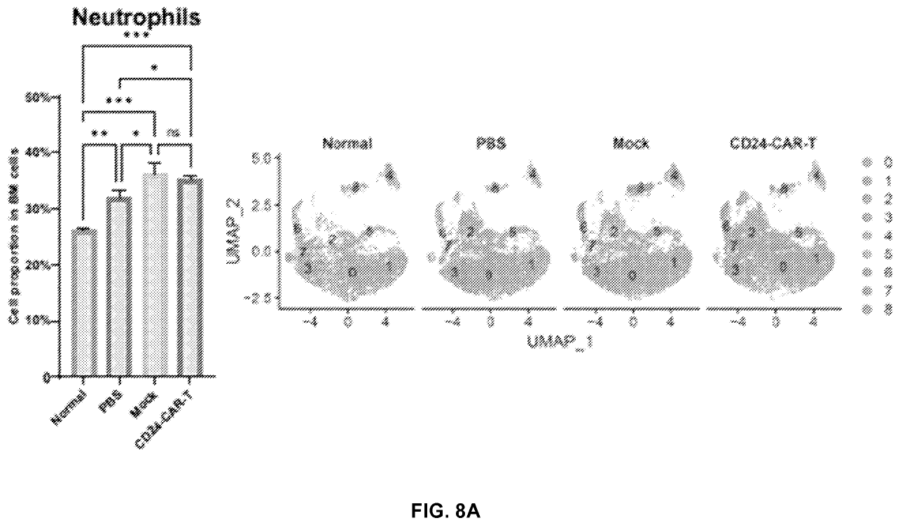

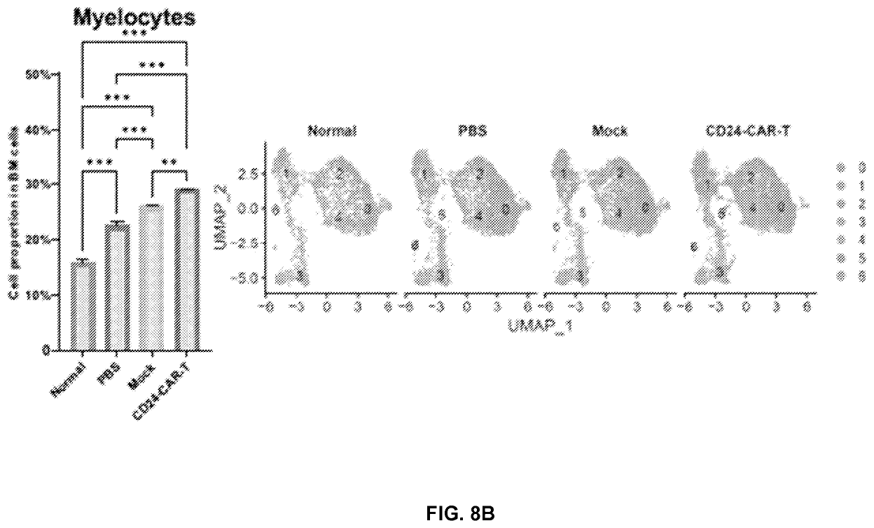

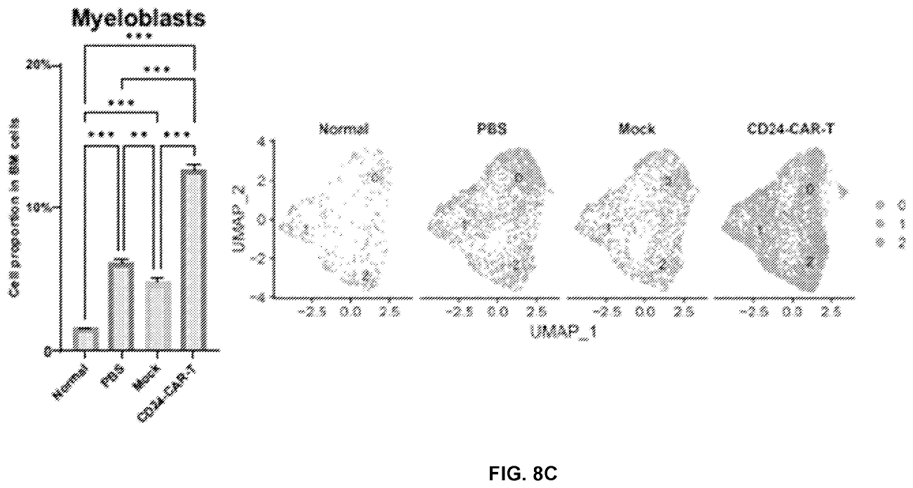

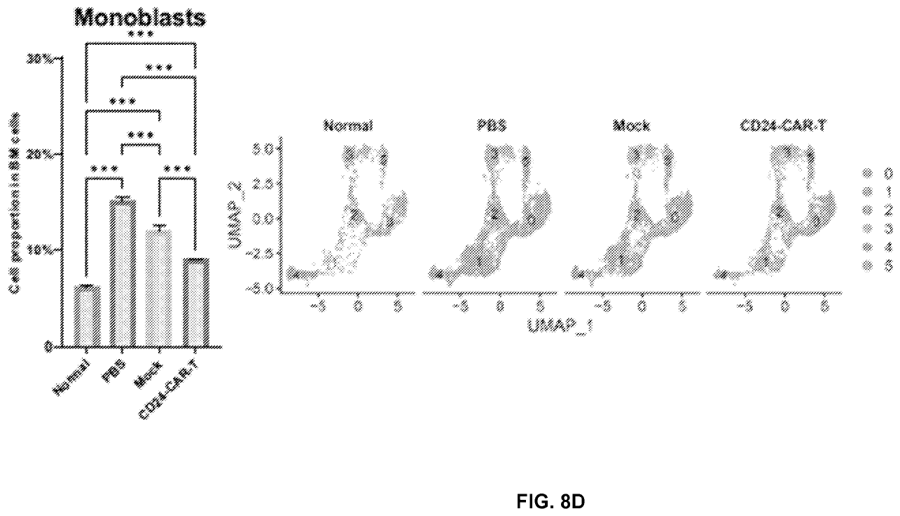

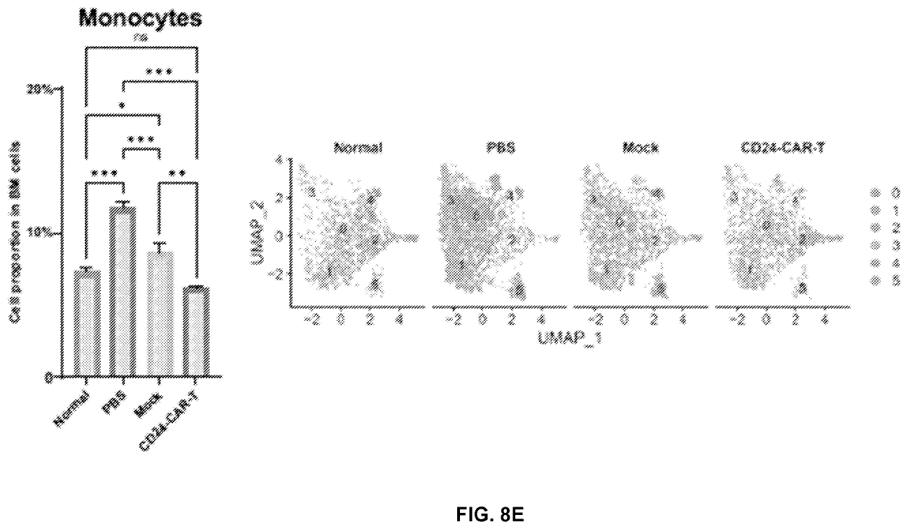

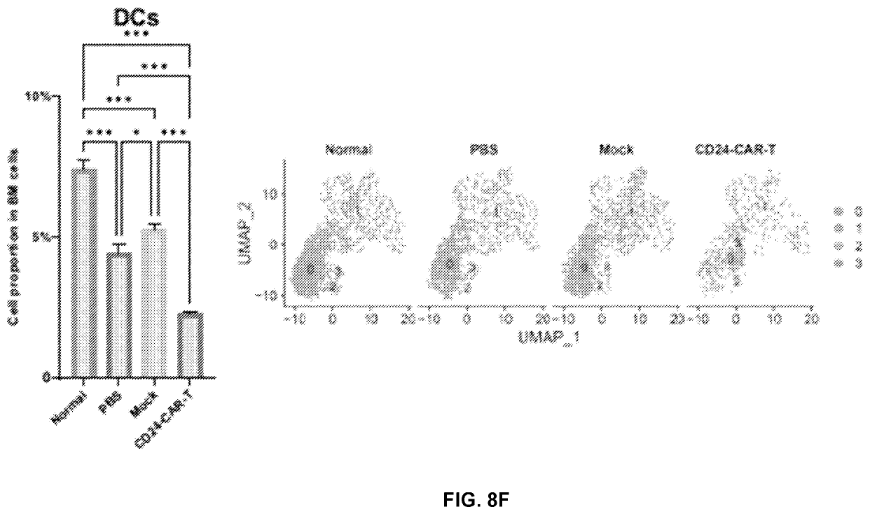

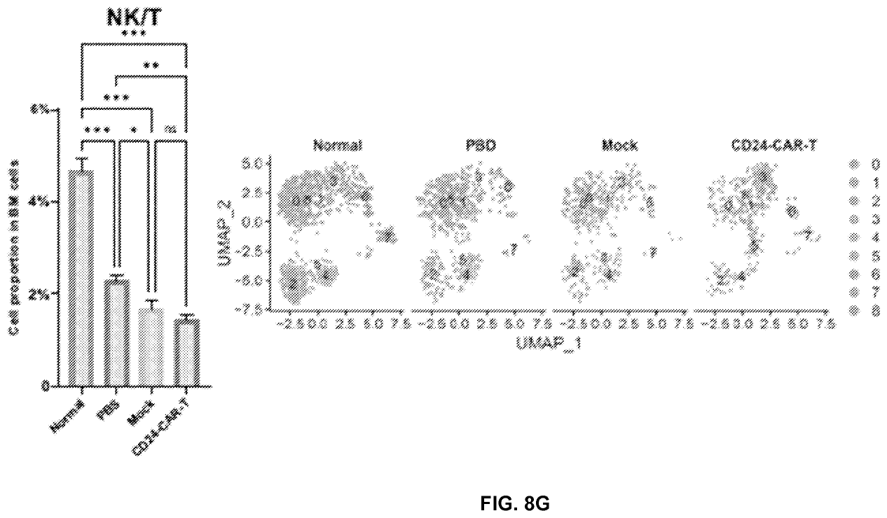

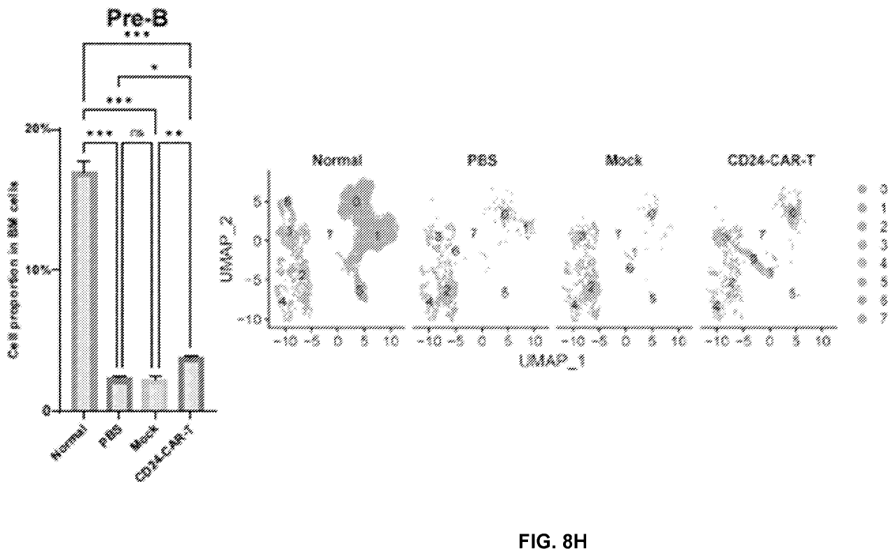

Next, the immune cell changes in the BM microenvironment of the 5TGM1 mouse model was analyzed using single-cell RNA sequencing (scRNA-seq). The Uniform Manifold Approximation and Projection (UMAP) plot identified 16 cell clusters based on genetic markers (FIG. 7A). The dot plot showed the expression of markers from different cell clusters (FIG. 7B). Split UMAP plot (FIG. 7C) and bar views of the quantitative proportion clusters (FIG. 7D and FIG. 8) of BM cells derived from the normal group (n=3), PBS group (n=3), MOCK-CAR-T group (n=3), or CD24-CAR-T group (n=3) identified several interesting cell cluster changes among the 4 groups. The series of neutrophil clusters (including its progenitors' myelocytes and myeloblasts clusters) increased in 5TGM1-bearing mice and continued to rise after CD24-CAR-T treatment. The series of macrophage clusters (including its progenitor's monoblast and monocyte clusters) was increased by more than 2.5-fold in tumor-bearing mice but was decreased to 80% of normal levels after treatment with CD24-CAR-T cells (FIG. 7E). Dendritic cells and natural killer (NK)/T cells declined in tumor-bearing mice and continued to decline after CD24-CAR-T cells treatment. Importantly, B cells decreased in tumor-bearing mice and increased after CD24-CAR-T cell treatment.

CD24-CAR-T cells clearly affect the function of macrophages (FIG. 7E) and block CD24/Siglec-10 signaling. The subclusters of macrophages were analyzed and it was observed that two subclusters were dramatically altered in mice treated with CD24-CAR-T cells compared to the controls (FIG. 7F). Cluster 0 was increased in MM-bearing mice, but decreased to normal levels after CD24-CAR-T cells treatment. Cluster 4 was a new cluster that appeared after CD24-CAR-T cells treatment. Differentially expressed genes were identified between these two clusters and compared those with the other clusters. The differentially expressed genes were divided into upregulated genes and downregulated genes. Kyoto Encyclopedia of Genes and Genomes (KEGG) pathway analysis was performed on these genes separately. Most of the signaling pathways that are upregulated (red bar) in Cluster 0 are tumor-associated or osteoclast-associated. Downregulated signaling pathways (blue bar) were associated with inflammation (FIG. 7G). Most of the signaling pathways that were upregulated (red bar) in Cluster 4 are inflammation- or phagocytosis-associated. Downregulated signaling pathways (blue bar) were tumor- or osteoclast-associated (FIG. 7H). These results indicated that CD24-CAR-T cells block the CD24-Siglec10 (“don't eat me” signal) pathway that allows macrophages to phagocytize tumor cells, and they also activated inflammation-associated signaling pathways. Due to the inflammatory reaction, the series of neutrophils (myelocytes, myeloblasts) was increased after CD24-CAR-T treatment.

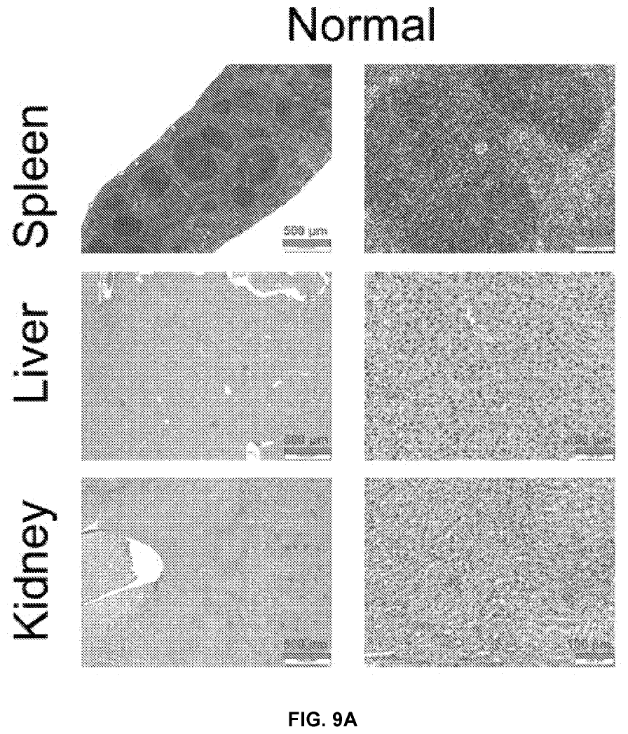

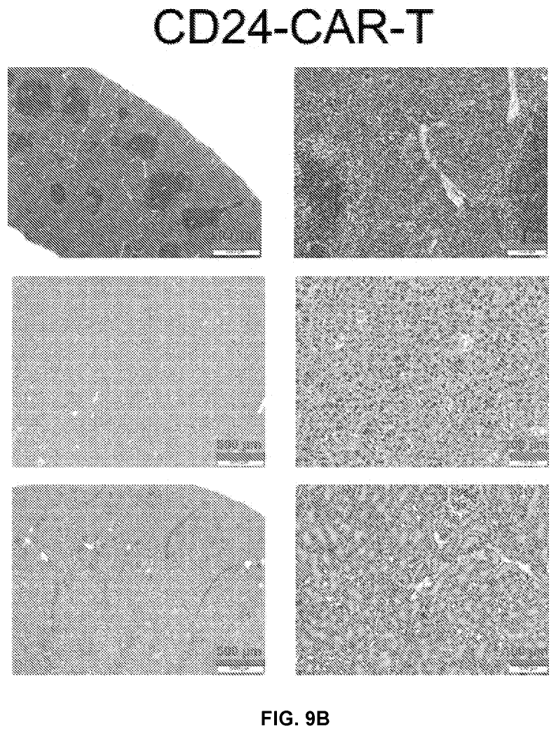

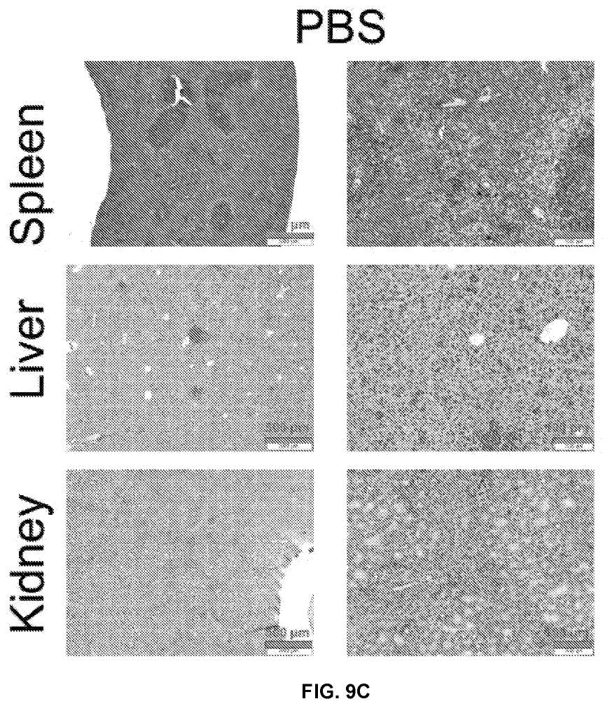

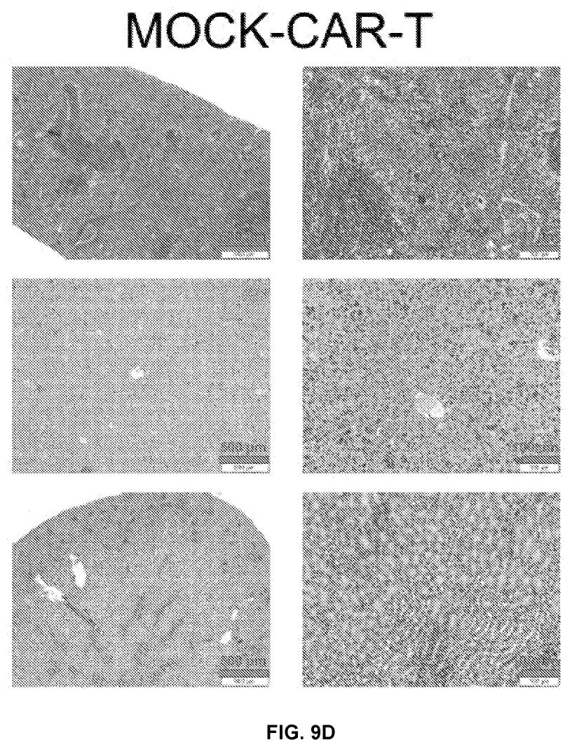

To determine if the increase in inflammation caused a cytokine storm that can result in tissue damage to mice, the spleen, liver, and kidney of the 4 groups were tested with hematoxylin and eosin (H&E) staining. Compared to the normal group, the CD24-CAR-T group did not experience appreciable organ damage (FIG. 9).

### Example 7-Bispecific BCMA-CD24-CAR-T Cells Exhibit Increased Cytolytic Activity In Vitro

Three types of bispecific BCMA-CD24-CAR T cells were constructed to target both the bulk tumor cells and TICs and further improve the antitumor efficacy of CAR-T cells: one dual-targeted CAR-T cell (Bi-CAR-T) and two tandem CAR-T cells (FIG. 10A). CD4/CD8 ratio and memory T-cell percentages were analyzed. The results showed that the proportion of CD4 Bi-CAR-T cells was 37.6% and CD8 was 53.8%, and the total percentage of CD4 stem cell memory (42.3%) and central memory (22.7%) was more than 60%. CD8 stem cell memory (17.5%) and central memory (44.1%) also occupied a large proportion of the T-cells (FIG. 10B).

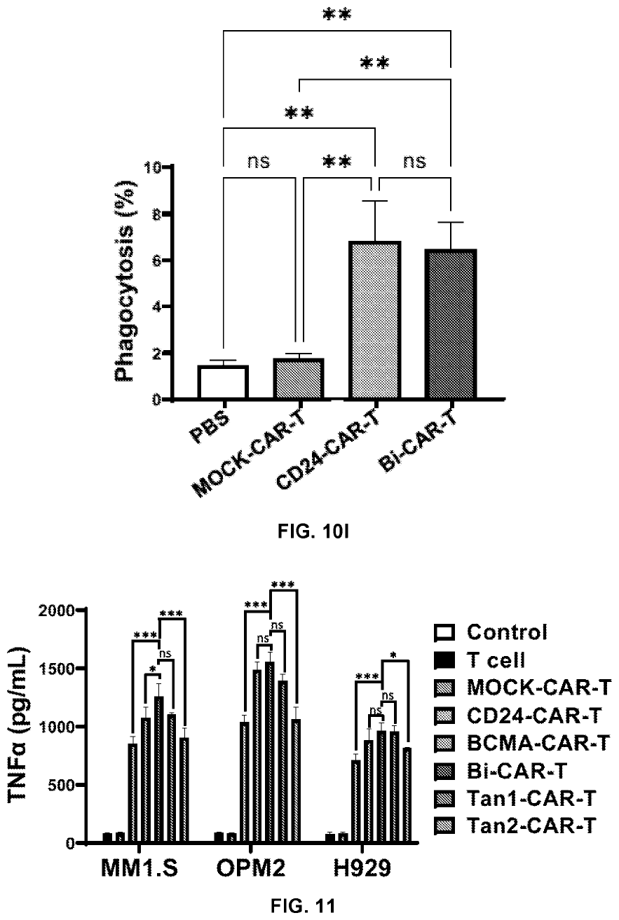

The cytolytic activity results showed that compared with other groups, the Bi-CAR-T cells had the highest cytolytic activity in vitro. When the Bi-CAR-T cell:MM ratio was 5:1, 87.3% of target cells were lysed in the MM1.S cell line, and there was a 4.4-fold increase in the percent lysis with Bi-CAR-T cells compared to the average of MOCK-CAR-T cells. In the OPM2 cell line, 94.2% of target cells were lysed, and there was a 7.1-fold increase in the percent lysis seen with Bi-CAR-T cells compared to the MOCK-CAR-T cells. In the H929 cell line, the percentage of target cells lysed was 93.0%, and there was a 7.9-fold increase in percent lysis seen with Bi-CAR-T cells compared to the MOCK-CAR-T cells (FIG. 10C). Bi-CAR-T cells also showed the highest activation (FIG. 10D); compared with other groups, Bi-CAR-T cells produced the highest levels of IFN-γ, IL-2, and TNF-α (FIG. 10E, FIG. 10F, and FIG. 11). For the clinical samples, the percentage of the subpopulation of CD138+ cells decreased by more than 42% in 16 of 16 primary myeloma samples after Bi-CAR-T treatment (FIG. 10G).

Compared with the MOCK-CAR-T cells and BCMA-CAR-T cells, the Bi-CAR-T cells strongly promoted phagocytic clearance by macrophages, similar to the CD24-CAR-T cells (FIG. 10H). Flow cytometry also showed the Bi-CAR-T cells significantly increased the phagocytic clearance by macrophages (FIG. 10I). Further, in vivo experiments were performed using Bi-CAR-T cells.

### Example 8-Bispecific BCMA-CD24-CAR-T Cells Prolong Mouse Survival

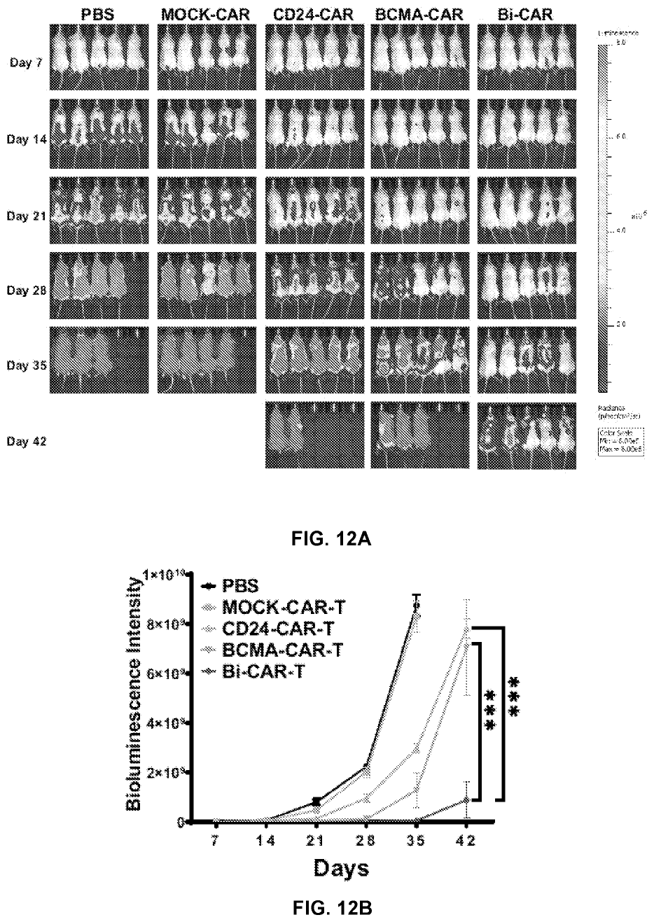

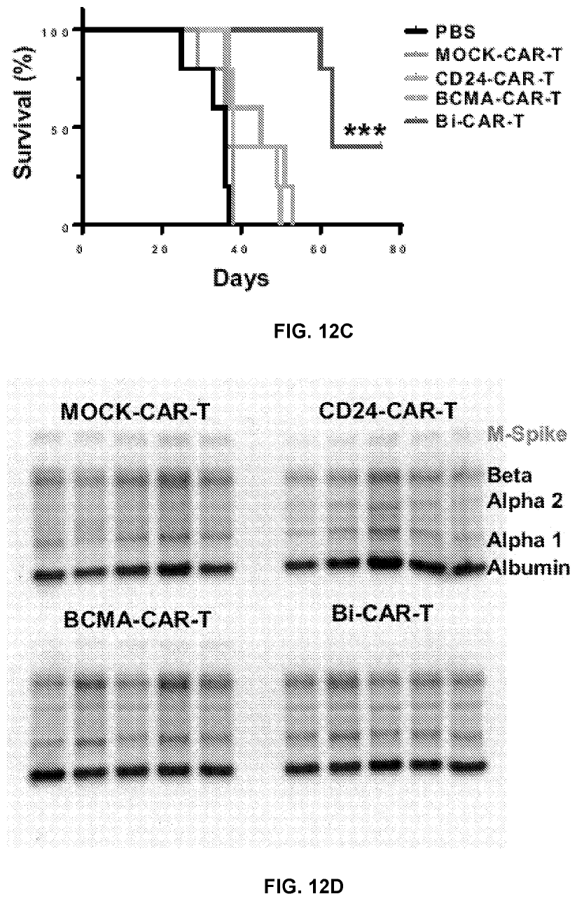

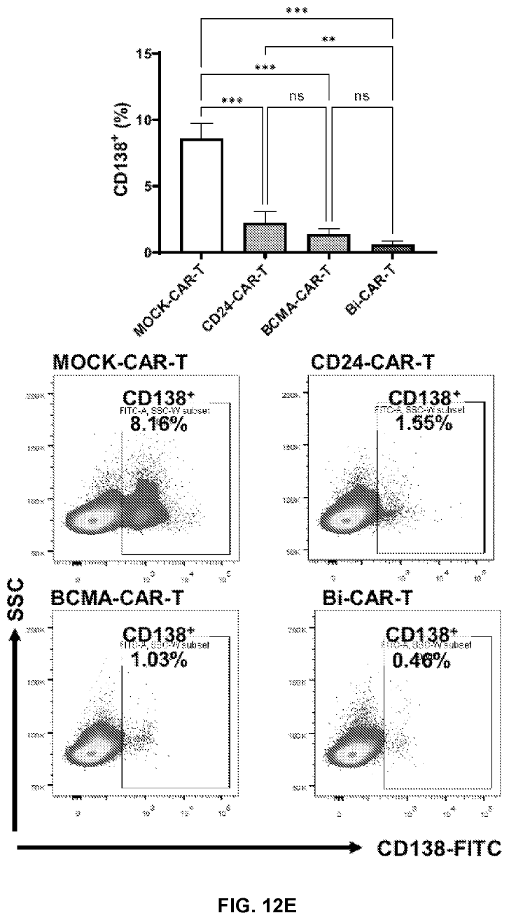

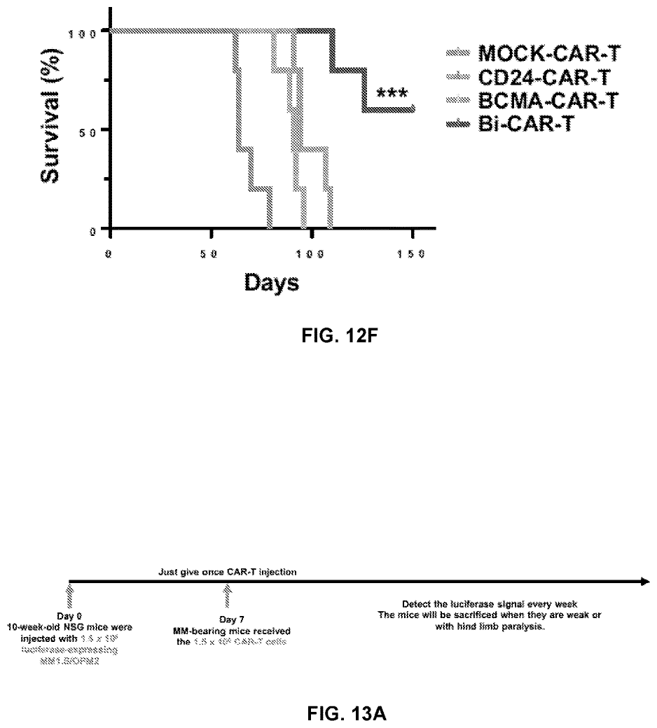

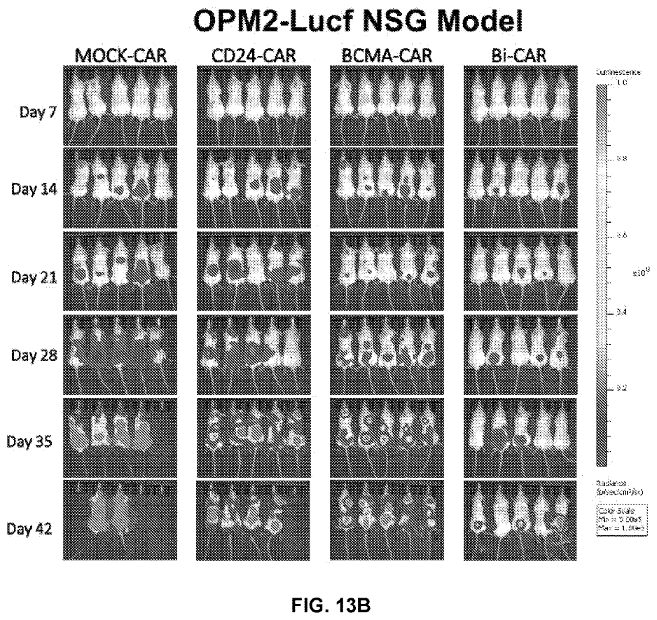

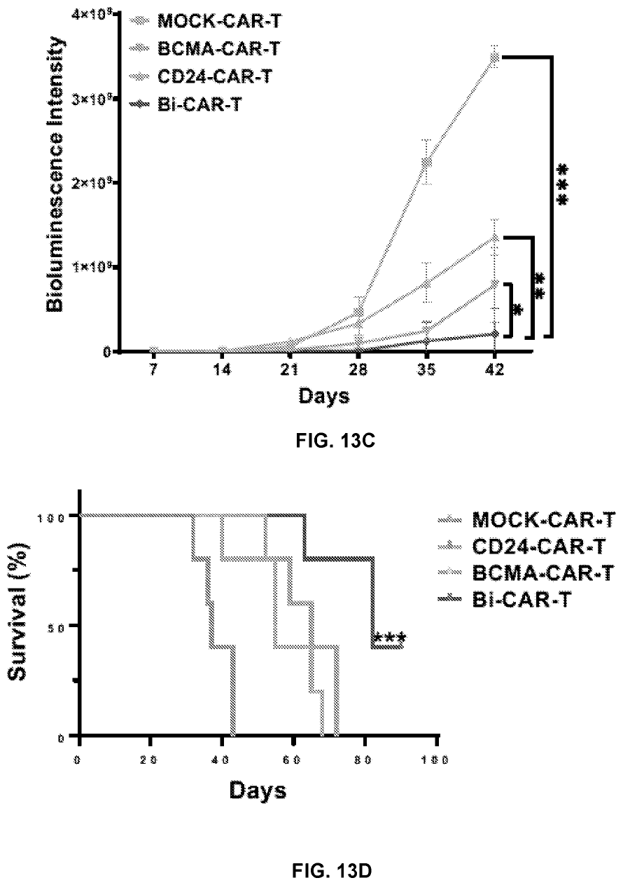

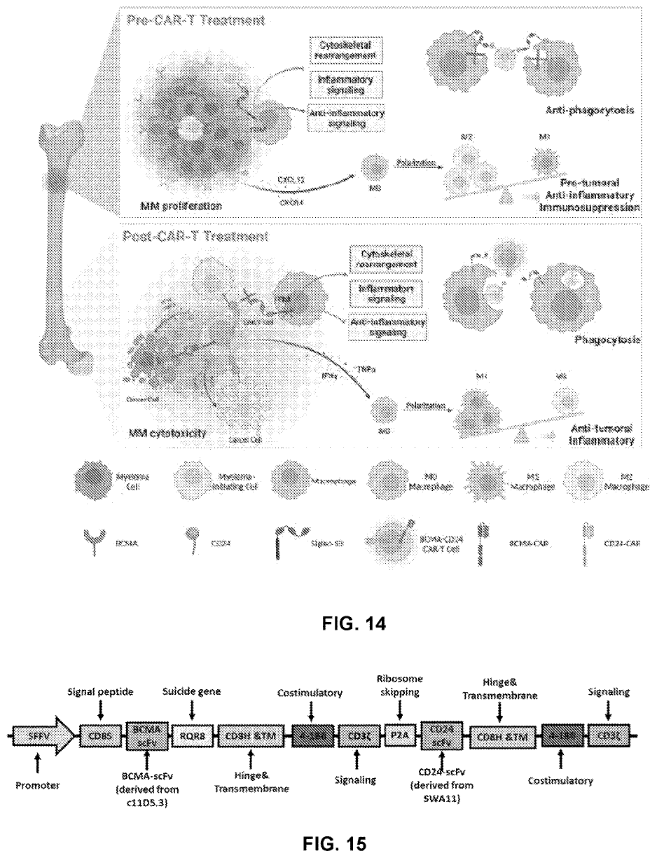

To detect human MM cell kill by CAR-T cells in vivo, immunodeficient NOD.Cg-Prkdcscid II2rg^{tm1Wjl}SzJ (NSG) mice were injected with MM1.S and OPM2 cells and subsequently treated with CAR-T cells or PBS. Bioluminescence imaging of MM1.S-bearing mice revealed Bi-CAR-T cells were the most effective in terms of antitumor activity, yielding near-complete tumor clearance by day 21 (FIG. 12A). Notably, animals treated with CD24-CAR-T cells had better tumor clearance than those treated with MOCK-CAR-T cells, but not as good as those treated with Bi-CAR-T cells (FIG. 12B). Additionally, median survival was more than 75% longer in the Bi-CAR-T group (63 days) than it was in the MOCK-CAR-T groups (36 days; p=. 0021; FIG. 12C). In the OPM2-bearing mice, Bi-CAR-T cells showed the best antitumor activity and the best survival (FIG. 13).

In the patient-derived xenograft mice, the M protein was significantly reduced in the group treated with Bi-CAR-T cells compared to the PBS group (FIG. 12D). Flow cytometry data indicated that BMMCs and human CD138⁺ MM cells were significantly decreased in the patient-derived xenograft mice after treatment with Bi-CAR-T cells compared to the patient-derived xenograft group (FIG. 12E). Patient-derived xenograft mice treated with Bi-CAR-T cells exhibited significantly increased survival compared to mice in all other groups (p<. 001; FIG. 12F).

### SUMMARY

Durable remissions following anti-B cell maturation antigen (BCMA)-specific chimeric antigen receptor (CAR) T-cell treatment of multiple myeloma (MM) are rare. Relapses occur when a small subset of MM tumor-initiating cells (TICs) survive treatment. MM cells expressing CD24 exhibit features of TICs. CD24-CAR-T cells were developed and tested their ability to eliminate MM TICs in vitro and in vivo. It was found that CD24-CAR-T cells potently block the CD24-Siglec-10 pathway, which promoted macrophage phagocytic clearance of MM cells. Additionally, CD24-CAR-T cells promote the polarization of macrophages from an M2 to M1 phenotype. A dual-targeted BCMA-CD24-CAR-T exhibited improved efficacy compared to monospecific BCMA-CAR-T-cell therapy.

Disclosed herein, are CD24-CAR-T cells against MM. The scFv from SWA11 was used for the CAR against CD24. CD24-CAR-T cells efficiently targeted dormant (GFP⁺DiD^{Hi}) cells in the 5TGM1 MM model. Simultaneously, CD24-CAR-T cells blocked the CD24-Siglec-10 pathway and promoted phagocytic clearance by macrophages. Inhibition of the CD24-Siglec-10 pathway also led to the activation of inflammatory signaling and antitumoral signaling. Additionally, it was found that M2ct were significantly increased in mice with 5TGM1 but decreased in mice with 5TGM1 treated with CD24-CAR-T. MM cells produce chemokines such as CXCL12, C-C chemokine ligand 2 (CCL2), and CD24⁺ cells upregulated CXCR4-positive macrophages towards the M2 phenotype. This phenomenon was reversed after CD24-CAR-T cell treatment. As MM cells were eliminated by CD24-CAR-T cells, the level of CXCL2 decreased. IFN-γ and TNFα released by activated CD24-CAR-T cells promoted macrophage polarization toward the M1Φ lineage. This reversed the immunosuppression caused by excessive M2Φ in the MM microenvironment.

To further improve antitumor efficacy, bispecific BCMA-CD24-CARs were constructed. To find optimal CAR signaling, three types of bispecific CAR-T cells were designed. Bi-CAR-T cells displayed superior activity in vitro and in vivo against MM cells. Bioluminescence imaging showed nearly complete tumor clearance in MM xenograft mouse models. Additionally, mice treated with Bi-CAR-T cells exhibited significantly increased survival compared to control mice in both the xenograft mouse model and the patient-derived xenograft model.

In CAR-T cell treatment, safety is one of the major challenges, including precise tumor targeting to avoid off-target or on-target/off-tumor toxicity. RQR8, an immunological safety switch was integrated as a suicide molecule into the design. It was found that CD24 was expressed in some normal cells, but the glycosylation of CD24 in normal cells is different from that in MM cells and could be used therapeutically to distinguish malignant MM cells from healthy cells.

Therefore, the disclosed BCMA-CD24-CAR-T cells effectively targeted bulk MM tumor cells, substantially reduced the numbers of tumor-initiating cells and modulated the tumor microenvironment.

TABLE 1
Sequence listing

| SEQ ID | | |
|---|---|---|
| NO | Type | Sequence |
| 1 | CD3Zeta | AGGGTGAAGTTCAGCAGGAGCGCCGACGCCCCCGCCTACCAGCAG |
| | nucleic acid | GGCCAGAACCAGCTGTACAACGAGCTGAACCTGGGCAGGAGGGAGG |
| | | AGTACGACGTGCTGGACAAGAGGAGGGGCAGGGACCCCGAGATGG |
| | | GCGGCAAGCCCCAGAGGAGGAAGAACCCCCAGGAGGGCCTGTACA |
| | | ACGAGCTGCAGAAGGACAAGATGGCCGAGGCCTACAGCGAGATCGG |
| | | CATGAAGGGCGAGAGGAGGAGGGGCAAGGGCCACGACGGCCTGTA |
| | | CCAGGGCCTGAGCACCGCCACCAAGGACACCTACGACGCCCTGCAC |
| | | ATGCAGGCCCTGCCCCCCAGG |
| 2 | CD3Zeta | RVKFSRSADAPAYQQGQNQLYNELNLGRREEYDVLDKRRGRDPEMGG |
| | amino acid | KPQRRKNPQEGLYNELQKDKMAEAYSEIGMKGERRRGKGHDGLYQGL |
| | | STATKDTYDALHMQALPPR |
| 3 | 4-1BB nucleic | AAACGGGGCAGAAAGAAACTCCTGTATATATTCAAACAACCATTTATG |
| | acid | AGACCAGTACAAACTACTCAAGAGGAAGATGGCTGTAGCTGCCGATT |
| | | TCCAGAAGAAGAAGAAGGAGGATGTGAACTG |
| 4 | 4-1BB amino | KRGRKKLLYIFKQPFMRPVQTTQEEDGCSCRFPEEEEGGCEL |
| | acid | |
| 5 | CD8 signal | ATGGCCTTACCAGTGACCGCCTTGCTCCTGCCGCTGGCCTTGCTGCT |
| | peptide | CCACGCCGCCAGGCCG |
| | nucleic acid | |
| 6 | CD8 signal | MALPVTALLLPLALLLHAARP |
| | peptide amino | |
| | acid | |
| 7 | CD24 scFv | GACGTGCACCTGCAGGAGAGCGGCCCCGACCTGGTGAAGCCCAGC |
| | nucleic acid | CAGAGCCTGAGCCTGACCTGCACCGTGACCGGCTACAGCATCACCA |
| | | GCGGCTACACCTGGCACTGGATCAGGCAGTTCCCCGGCAACACCGT |
| | | GGAGTGGATGGGCTACATCCAGTACACCGGCAGCACCAGGTACAAC |
| | | CCCGCCCTGAGGGGCAGGCTGAGCATCAGCAGGGACACCAGCAAG |
| | | AACCAGTTCTTCCTGCAGCTGATCAGCGTGACCACCGCCGACACCG |
| | | GCACCTACTTCTGCGCCAGGGGCACCACCGCCAGCTTCGACTACTG |
| | | GGGCCAGGGCACCACCCTGACCGTGGCCAGCGCCGGCGGCGGCG |
| | | GCAGCGGCGGCGGCGGCAGCGGCGGCGGCGGCAGCGACATCGTG |
| | | ATGAGCCAGAGCCCCAGCAGCCTGAACGTGAGCGTGGGCGAGAAG |
| | | GTGACCATGAGGTGCAGGAGCAGCCAGAGCCTGCTGTACAGCAGCG |
| | | ACCAGAAGAACTACCTGACCTGGTACCAGCAGAAGCCCGGCCAGAG |
| | | CCCCAAGCTGCTGATCAGCTGGGCCAGCACCAGGGCCAGCGGCGT |
| | | GCCCGACAGGTTCACCGGCAGCGGCAGCGGCACCGACTTCACCCTG |
| | | ACCATCAGCAGCGTGAAGGCCGAGGACCTGGGCGTGTACTACTGCC |
| | | AGCAGTACTTCATCTACCCCCTGACCTTCGGCGTGGGCACCAAGCTG |
| | | GGCCTGAAG |
| 8 | CD24 scFv | DVHLQESGPDLVKPSQSLSLTCTVTGYSITSGYTWHWIRQFPGNTVEW |
| | amino acid | MGYIQYTGSTRYNPALRGRLSISRDTSKNQFFLQLISVTTADTGTYFCAR |
| | | GTTASFDYWGQGTTLTVASAGGGGSGGGGSGGGGSDIVMSQSPSSLN |
| | | VSVGEKVTMRCRSSQSLLYSSDQKNYLTWYQQKPGQSPKLLISWASTR |
| | | ASGVPDRFTGSGSGTDFTLTISSVKAEDLGVYYCQQYFIYPLTFGVGTKL |
| | | GLK |
| 9 | Hinge & | ACCACGACGCCAGCGCCGCGACCACCAACACCGGCGCCCACCATCG |
| | Transmembrane | CGTCGCAGCCCCTGTCCCTGCGCCCAGAGGCGTGCCGGCCAGCGG |
| | nucleic | CGGGGGGCGCAGTGCACACGAGGGGGCTGGACTTCGCCTGTGATAT |
| | acid | CTACATCTGGGCGCCCTTGGCCGGGACTTGTGGGGTCCTTCTCCTGT |
| | | CACTGGTTATCACCCTTTACTGC |
| 10 | Hinge & | TTTPAPRPPTPAPTIASQPLSLRPEACRPAAGGAVHTRGLDFACDIYIWA |
| | Transmembrane | PLAGTCGVLLLSLVITLYC |
| | amino acid | |
| 11 | Suicide gene | GCCTGCCCCTACAGCAACCCCAGCCTGTGCAGCGGCGGCGGCGGC |
| | nucleic acid | AGCGAGCTGCCCACCCAGGGCACCTTCAGCAACGTGAGCACCAACG |
| | | TGAGCCCCGCCAAGCCCACCACCACCGCCTGTCCTTATTCCAATCCT |
| | | TCCCTGTGTAGCGGCGGCGGCGGCAGC |
| 12 | Suicide gene | ACPYSNPSLCSGGGGSELPTQGTFSNVSTNVSPAKPTTTACPYSNPSLC |
| | amino acid | SGGGGS |
| 13 | P2A nucleic | GCCACCAACTTAGCCTGCTGAAGCAGGCCGGCGACGTGGAGGAGA |
| | acid | ACCCGGCCCC |
| 14 | P2A amino | ATNFSLLKQAGDVEENPGP |
| | acid | |
| 15 | SFFV | GTAACGCCATTTTGCAAGGCATGGAAAAATACCAAACCAAGAATAGA |
| | promoter | GAAGTTCAGATCAAGGGCGGGTACATGAAAATAGCTAACGTTGGGCC |
| | nucleic acid | AAACAGGATATCTGCGGTGAGCAGTTTCGGCCCCGGCCCGGGGCCA |
| | | AGAACAGATGGTCACCGCAGTTTCGGCCCCGGCCCGAGGCCAAGAA |
| | | CAGATGGTCCCCAGATATGGCCCAACCCTCAGCAGTTTCTTAAGACC |
| | | CATCAGATGTTTCCAGGCTCCCCCAAGGACCTGAAATGACCCTGCGC |
| | | CTTATTTGAATTAACCAATCAGCCTGCTTCTCGCTTCTGTTCGCGCGC |
| | | TTCTGCTTCCCGAGCTCTATAAAAGAGCTCACAACCCCTCACTCGGC |
| | | GCGCCAGTCCTCCGACAGACTGAGTCGCCCGGG |
| 16 | BCMA scFv | GACATCGTGCTGACCCAGAGCCCCCCCAGCCTGGCCATGAGCCTGG |
| | nucleic acid | GCAAGAGGGCCACCATCAGCTGCAGGGCCAGCGAGAGCGTGACCAT |
| | | CCTGGGCAGCCACCTGATCCACTGGTACCAGCAGSAGCCCGGCCAG |
| | | CCCCCCACCCTGCTGATCCAGCTGGCCAGCAACGTGCAGACCGGCG |
| | | TGCCCGCCAGGTTCAGCGGCAGCGGCAGCAGGACCGACTTCACCCT |
| | | GACCATCGACCCCGTGGAGGAGGACGACGTGGCCGTGTACTACTGC |
| | | CTGCAGAGCAGGACCATCCCCAGGACCTTCGGCGGCGGCACCAAGC |
| | | TGGAGATCAAGGGAGGGGGGGGATCCGGGGGAGGAGGCTCGGCG |
| | | GAGGCGGAAGCCAGATCCAGCTGGTGCAGAGCGGCCCCGAGCTGA |
| | | AGAAGCCCGGCGAGACCGTGAAGATCAGCTGCAAGGCCAGCGGCTA |
| | | CACCTTCACCGACTACAGCATCAACTGGGTGAAGAGGGCCCCCGGC |
| | | AAGGGCCTGAAGTGGATGGGCTGGATCAACACCGAGACCAGGGAGC |
| | | CCGCCTACGCCTACGACTTCAGGGGCAGGTTCGCCTTCAGCCTGGA |
| | | GACCAGCGCCAGCACCGCCTACCTGCAGATCAACAACCTGAAGTAC |
| | | GAGGACACCGCCACCTACTTCTGCGCCCTGGACTACAGCTACGCCAT |
| | | GGACTACTGGGGCCAGGGCACCAGCGTGACCGTGAGCAGC |
| 17 | BCMA scFv | DIVLTQSPPSLAMSLGKRATISCRASESVTILGSHLIHWYQQKPGQPPTLL |
| | amino acid | IQLASNVQTGVPARFSGSGSRTDFTLTIDPVEEDDVAVYYCLQSRTIPRT |
| | | FGGGTKLEIKGGGGSGGGGSGGGGSQIQLVQSGPELKKPGEETVKISCK |
| | | ASGYTFTDYSINWVKRAPGKGLKWMGWINTETREPAYAYDFRGRFAFS |
| | | LETASTAYLQINNLKYEDTATYFCALDYSYAMDYWGQGTSVTVSS |
| 18 | CD24 amino | MGRAMVARLGLGLLLLALLLPTQIYSSETTTGTSSNSSQSTSNSGLAPNP |
| | acid | TNATTKAAGGALQSTASLFVVSLSLLHLYS |

What is claimed is:

1. An immune effector cell comprising a chimeric antigen receptor, wherein chimeric antigen receptor comprises a first antigen recognition domain which binds to protein selected from the group consisting of CD24 and B-cell maturation antigen (BCMA).

2. The immune effector cell of claim 1, wherein the first antigen recognition domain which binds to CD24.

3. The immune effector cell of claim 2, wherein the chimeric antigen receptor further comprises a second antigen recognition domain.

4. The immune effector cell of claim 3, wherein the second antigen recognition domain specifically binds to B-cell maturation antigen (BCMA).

5. The immune effector cell of claim 3, wherein the first antigen and second antigen recognition domain are in tandem.

6. The immune effector cell of claim 1, wherein the first antigen recognition domain binds to BCMA.

7. The immune effector cell of claim 6, wherein the chimeric antigen receptor further comprises a second antigen recognition domain.

8. The immune effector cell of claim 7, wherein the second antigen recognition domain specifically binds to CD24.

9. The immune effector cell of claim 7, wherein the first antigen recognition domain, and the second antigen recognition domain further comprise a transmembrane domain, an intracellular domain, or any combination thereof.

10. The immune effector cell of claim 1, wherein the antigen recognition domain which binds to CD24 comprises an scFv with a nucleic acid sequence having at least 70%, 80%, 90%, or 100% sequence identity with SEQ ID NO: 7 or an amino acid sequence having at least 70%, 80%, 90%, or 100% sequence identity with SEQ ID NO: 8.

11. The immune effector cell of claim 1, wherein the antigen recognition domain which binds to BCMA comprises an scFv with a nucleic acid sequence having at least 70%, 80%, 90%, or 100% sequence identity with SEQ ID NO: 16 or an amino acid sequence having at least 70%, 80%, 90%, or 100% sequence identity with SEQ ID NO: 17.

12. The immune effector cell of any one of the preceding claim 1, wherein the chimeric antigen receptor further comprises a suicide gene.

13. The immune effector cell of claim 12, wherein the suicide gene comprises a RQR8, a modified RQR8 comprising at least one CD20 mimotope, at least one CD34 epitope, or any combination thereof.

14. (canceled)

15. The immune effector cell of claim 1, wherein an immune effector cell is selected from the group consisting of a T cell, a Natural Killer (NK) cell, a cytotoxic T lymphocyte (CTL), and a regulatory T cell, and any combination thereof.

16. A composition comprising an immune effector cell or a population of immune effector cell of claim 1, and a pharmaceutically acceptable carrier.

17. A method of treating a malignancy in a subject in need thereof, the method comprising administering to the subject the composition of claim 16.

18. The method of claim 17, wherein the malignancy is a B cell malignancy or a plasma cell malignancy.

19. The method of claim 17, wherein the malignancy is a Burkitt lymphoma, chronic lymphocytic leukemia/small lymphocytic lymphoma (CLL/SLL), diffuse large B-cell lymphoma, follicular lymphoma, or mantle cell lymphoma, Monoclonal gammopathy of undetermined significance, Lymphoplasmacytic lymphoma, Plasmacytoma, or myeloma.

20. (canceled)

21. The method of claim 18, wherein the malignancy is a relapsed or refractory malignancy.

22. The method of claim 17, wherein administering the population of immune effector cell: (a) reduces the tumor burden in the subject by at least 10%; or (b) increases survivability of the subject by at least 10%.

23. (canceled)
